SEQUENCE LISTING (76 sequences)

SEQ ID NO: 1 - DNA, 6374 bp, Homo sapiens
acgtctgtggcgccctcgcaccgcgccgcagccatggccctggtgaccctgcagcgctcgcccacgcccagcgccgcctcctcctcggccagcaacagcgagttggaggctggcagcgaagaagatcgaaaattaaacctcagcttaagtgagagctttttcatggtgaaaggcgcagccctcttcttacaacagggaagcagccctcaaggccagcggagtcttcagcacccccacaagcatgcaggtgatctgcctcaacatcttcaggtgatgatcaaccttctgcgttgcgaagacagaatcaagctggcagtgcgcctggagagcgcctgggcggaccgggtccggtacatggtggtggtgtacagcagcgggcgccaggacaccgaggagaatatcttgctgggagtggacttttccagtaaggaaagtaaaagctgcaccattgggatggttctccgactgtggagcgacacgaaaatccaccttgatggagatggtgggttcagcgtgagcacagcaggaaggatgcacatatttaagcctgtgtctgtccaggccatgtggtctgccctgcaggtgcttcacaaggcctgcgaagtggcccggaggcacaactacttccccgggggtgtagctctcatctgggctacctactatgagagctgcatcagctccgagcagagctgcatcaacgagtggaacgccatgcaggacctggagtctacgcggcccgactcccccgcgctatttgtggacaagcccactgaaggggaaaggaccgagcgcctcatcaaagccaagctccgaagcatcatgatgagccaggatctagaaaatgtgacttccaaagagattcgtaatgaattagagaaacagatgaattgtaacttgaaggaactcaaggaatttatagacaatgagatgctacttatcttgggacagatggacaagccctcccttatcttcgatcatctttatctcggctctgaatggaatgcatccaatctggaggaactgcagggctcaggggttgattacattttaaatgttaccagagaaatcgataatttttttcctggcttatttgcatatcataacatccgagtctacgatgaagagaccacagacctcctcgcccactggaatgaagcgtatcattttataaacaaagcgaagaggaaccattccaagtgcctggtgcattgcaaaatgggcgtgagtcgctcggcctccacagtcatagcctatgcaatgaaggaattcggctggcctctggaaaaagcatataactatgtaaagcagaagcgcagcatcacgcgccccaacgcgggctttatgaggcagctgtctgagtatgaaggcatcttggatgcaagcaaacagcggcacaacaagctgtggcgtcagcagacagacagcagcctccagcagcctgtggatgaccctgcaggacctggcgacttcttgccagagaccccagatggcaccccggaaagccagctgcccttcttggatgatgccgcccagcccggcttagggccccccctcccctgctgtttccggcgactctcagacccccttctgccttcccctgaggatgaaactggcagcttggtccacctggaggatccggagagggaggctctgttggaggaagctgctccacctgcagaggtgcacaggccggccagacagccccagcaaggttccggactctgtgagaaggatgtgaagaagaaactagagtttgggagtcccaaaggtcggagcggctccttgctgcaggtggaggagacggaaagggaggagggcctgggagcagggaggtgggggcagcttccaacccagctcgatcaaaacctgctcaactcggagaacctaaacaacaacagcaagaggagctgtcccaacggcatggaggatgatgctatatttgggatccttaacaaagtgaagccttcctataaatcctgtgccgactgcatgtaccctacagccagcggggctcctgaggcctccagggagcgatgtgaggaccccaatgctcccgccatctgcacccagccagccttcctaccccacatcacgtcctcccctgtggcccacttggccagcaggtcccgtgttccggagaagccagcctctggcccaaccgaacctcccccgttcctaccaccagcaggctccaggagggcagacaccagtggccctggggctggagctgccctagagccaccagccagccttttggaaccttccagagagaccccaaaagtcctgccaaagtccctccttttgaagaattctcactgtgataagaaccctcccagcacagaagtggtaataaaggaagaatcgtcacccaagaaagatatgaagccagccaaggacctgaggcttctgttcagtaatgaatctgagaagccgacaaccaacagctacctgatgcagcaccaggagtccatcattcagctgcagaaggcaggcttggtccgcaagcacaccaaagagctagagcggctgaagagcgtgcctgcagacccagcacctccctccagggatggccctgccagcaggctggaggccagcatccccgaggagagccaggatccagccgcgctccacgagctgggccccctggttatgcccagccaggccgggagtgatgagaagtcagaggccgcccccgcttcattggaaggaggctcactgaagagcccccctcctttcttctaccgcctggaccacaccagtagtttctcaaaagactttctgaagaccatctgctacacccccacctcctcttccatgagctccaacctgacccggagctccagcagcgatagcatccacagtgtccgtgggaagcccgggctggtgaagcagcggacacaggagattgagacccggctccggctggcgggcctcaccgtctcttccccactgaagcgctcacactctcttgccaagctggggagtctcaccttctcaacggaagacctgtccagtgaggctgacccgtccaccgtcgctgactcccaggacaccactttgagtgaatcttccttcttgcatgagccccagggaaccccgagggacccagctgcaacctccaaaccatcagggaaacccgccccagaaaacttaaagagcccttcgtggatgagcaaaagctgacccgccttttgctgcgtcaggctgggcggatattttcatatgatccctccctcactttcactgtggatttggatcgaccccttacatcttgatttcccttaaacgagtgaagtcatcctaacttttcccctactcttgccatagagaggaggagaagaaactaaccaacatgcagcacaagaaggggagctgtcagtcgtgtggcctgggagaaccaggagccgccggccaggagacacaaaactccgctcccaccgtgtcttcaagaagaaccatgttttaggaagaacgtgcacacataggcgcacacatccagactgttcccttcgcatcctgcagaagagtcctggtggtggcagcctcaccgcggcacacgttctagctcattcttggcctcgcagaaaactctcggatggcaacattaagtcctacctctgttctcgctgcgctttttatttttaaaacacacgaatgaccaaggcttgttccagggaataatgctctgtctgaaagcgtttttccaaaaaaagaatttttcgttttgtgttttggttgggaccccagacgtgtcaagacttttaacccgacagccccaagcactgctgagtaaagtcatcagatgagtagtcccacgctgttgttgagctgactctgtgcccgaatggtggtgcaggcgccctgtcctggttgtgttgttacgtgtttgaccagcacaggggtccggtggggaaaatgtatgggttgttctcagttgttgctaatgctgaagtttaaatctcaaggggaagggcccacctgcattgttgagtgtctgctgctgaaacacatgattgtgtttaggtttgaaattgctcaagtgtctggctcaggtggtggttctgagacacatcgtcctgctgagagcccagatgcttaggtccactagggcccatctagggaagggaaaggagatttcagcggcttccccgaaaggaacgggactgtcgggatgcttcccggatgtctacagttgccccttcctgcagtgagattactgcttcctgtttccctccagctcttcccagcagcagtgagggagtataagagggatctgtagtcgctgcctggctctgtgggcggcccctttaagactcaggtgagctcagccagtcccgctgcgccaggcttgaatcaaaggtgtcagcaagctagatgtcaaaacatttcgagagagaggcagcttgcagaaaagcagaaaggttgaattcaggggtcagcaaactatggcatgtggccccgtgagctaggaatggtttttcctctttaagacagggcctggctctgtcacctaggctggagtacagtagtgcaatcatgactcactgcagcatcaacctccctggctcaagcgatggcttttacctttttaaggagttgtaaaaaaaagaaataatatgtgacagaattcatgtggcccacaaagcctgaggtacttactgtcaggccctttgtagaaaaagttgctgacccctggtttgatttgccacacagggctgctcatggccctgtaggaagaaactgggtaaatgtgaagccctctgcctctctgacacctaaggccagacccttccctcctcccgaccacaagctttgccagccccgcactagcctcacctgtgcaggatggagtaggttgcgtacatgggggaaggcaggtgaacacagttggctgaagctcttccacaattccatcttgccctcagctgggtccgccagattaactcagtgaaaccagaaagccttcaaggaccagctgaattctgaaagtgagtgagtgagccatcatcttaacattggccagaactgtgttccccaaagctattctagaaagcaccccaggagggatctgcaggaacaaggctagttcatattttacctagtgagcacagtttttgcaaaaatcctccctccaggactttgtctccttggagtgatttttaaaaatacatacattactttatagggagctgttttcccactagtgtcaattaaaatcaccttaaaaggtgattatccacttattcctaaacccctgtgggttgttccccctttccctcagccaacaaaagcatagcctcaaaaaatatcaagttcggtatgttttgccaaatcaaatttcatgtggtagatcaatttttgtgtcaaaataatcttttaaatttagtgatgacaggcttttgttggttttttaaccacgtctatgtatgagaatgatatatttttgaaaactttaattttgaaagccataatttttcttatctaaagagttggggggtggggtgtggaatctggagagtacaagttggtctttggcttctggcaaacttacccattcatttttggaagcacagctagcatatcaacatccagacgagagcgctggtcccgtccacagagcagagtgaagcattctggacttgatgcttaatagcctggcctggagaaaagggtaaggtttatttttggaaacccagatcagttgcatgtaaacagatggcacatggctatttaaaatgctgtatgatggggccgggcgcggtggctcacgcctgtaatcctagcactttgggaggccgaggcaggcggatcatctgaggtcaggagttctagaccagcctggccaacatggtgaaaccccgtctctactaaaaatacaaaaacttagctgggtgtggtagtgcgtgcctgtaatcctagctacttaggaggctgaggcaggagaatcgcttgaacctgggtggcggaggttgcaatgagccgagatcgcgccactgcactccagcctgggtgcaatgaaactgggagtgaaactccgtctcaaaaaaataaaatgctgtatgaacaagatgagcattctgtcaggtgtcgggacacctgggcaaagacgaattcatgctgtctgtgaaaaggaagtttgcactgtaacatatgccatagcttggcccttgctttgtatgcaaccttagctgatggggaaaattccaaacataagtggcacaggaaagaggcaggcagaggaggcaggggtctgtgctgtgcaagtcactggttttttatgactatcattttcattgaatgcatttgttgaattgggacaaaaggaacattttctaaatcagcttgatactctttataaaaaacagctgaatctt

SEQ ID NO: 2 - DNA, 2732 bp, Homo sapiens
gaaaagaagacgaggaggagagcgacgggacgggacgcgagcgggagcgcagccgccctctcggctccgcggcggcgcctcgcaagtccgggaggcgaggggggcccgaggggagacgccgtgacaactttcgtttccctctgagggaattgggaggtcggcggccccaaaagctttcagtccagtgtaaagctgttggagcgcgggagcaaaggtaaagaatgatgtaatgcgctggctgctccaaagcatcttttgttgtggaatggttattccagtcatctctttatgaatcaaatgtgaggggctgctttgtggacggagtcctttgcaagagcacatcaacgggaaagagaaagagacattcacttggagggctcttgctgaaaatgggtttaactctccttttgccagtcaccaccagcctgacctcatacacttttagtacaatggagtggctgagcctttgagcacaccaccattacatcatcgtggcaaattaaagaaggaggtgggaaaagaggacttattgttgtcatggcccatgagatgattggaactcaaattgttactgagaggttggtggctctgctggaaagtggaacggaaaaagtgctgctaattgatagccggccatttgtggaatacaatacatcccacattttggaagccattaatatcaactgctccaagcttatgaagcgaaggttgcaacaggacaaagtgttaattacagagctcatccagcattcagcgaaacataaggttgacattgattgcagtcagaaggttgtagtttacgatcaaagctcccaagatgttgcctctctctcttcagactgttttctcactgtacttctgggtaaactggagaagagcttcaactctgttcacctgcttgcaggtgggtttgctgagttctctcgttgtttccctggcctctgtgaaggaaaatccactctagtccctacctgcatttctcagccttgcttacctgttgccaacattgggccaacccgaattcttcccaatctttatcttggctgccagcgagatgtcctcaacaaggagctgatgcagcagaatgggattggttatgtgttaaatgccagcaatacctgtccaaagcctgactttatccccgagtctcatttcctgcgtgtgcctgtgaatgacagcttttgtgagaaaattttgccgtggttggacaaatcagtagatttcattgagaaagcaaaagcctccaatggatgtgttctagtgcactgtttagctgggatctcccgctccgccaccatcgctatcgcctacatcatgaagaggatggacatgtctttagatgaagcttacagatttgtgaaagaaaaaagacctactatatctccaaacttcaattttctgggccaactcctggactatgagaagaagattaagaaccagactggagcatcagggccaaagagcaaactcaagctgctgcacctggagaagccaaatgaacctgtccctgctgtctcagagggtggacagaaaagcgagacgcccctcagtccaccctgtgccgactctgctacctcagaggcagcaggacaaaggcccgtgcatcccgccagcgtgcccagcgtgcccagcgtgcagccgtcgctgttagaggacagcccgctggtacaggcgctcagtgggctgcacctgtccgcagacaggctggaagacagcaataagctcaagcgttccttctctctggatatcaaatcagtttcatattcagccagcatggcagcatccttacatggcttctcctcatcagaagatgctttggaatactacaaaccttccactactctggatgggaccaacaagctatgccagttctcccctgttcaggaactatcggagcagactcccgaaaccagtcctgataaggaggaagccagcatccccaagaagctgcagactgccaggccttcagacagccagagcaagcgattgcattcggtcagaaccagcagcagtggcaccgcccagaggtcccttttatctccactgcatcgaagtgggagcgtggaggacaattaccacaccagcttccttttcggcctttccaccagccagcagcacctcacgaagtctgctggcctgggccttaagggctggcactcggatatcttggccccccagacctctaccccttccctgaccagcagctggtattttgccacagagtcctcacacttctactctgcctcagccatctacggaggcagtgccagttactctgcctacagctgcagccagctgcccacttgcggagaccaagtctattctgtgcgcaggcggcagaagccaagtgacagagctgactcgcggcggagctggcatgaagagagcccctttgaaaagcagtttaaacgcagaagctgccaaatggaatttggagagagcatcatgtcagagaacaggtcacgggaagagctggggaaagtgggcagtcagtctagcttttcgggcagcatggaaatcattgaggtctcctgagaagaaagacacttgtgacttctatagacaatttttttttcttgttcacaaaaaaattccctgtaaatctgaaatatatatatgtacatacatatatatttttggaaaatggagctatggtgtaaaagcaacaggtggatcaacccagttgttactctcttaacatctgcatttgagagatcagctaatacttctct

SEQ ID NO: 3 - DNA, 2260 bp, Homo sapiens
ggggggaaaagttaagaaaaagcccccgagagccggggtgaagggagtaaactggtctagcccagttctgtctgcgcccagtgagagggtttgaaactccgcggagccctttcccaatagaaaacgtgtttgcttcaggattttcatctcagctgcttttttttaaggttggttaagctgtgcgccgccccacctcataaaatggttgtctctcaggagtgaagactccagcggccccccgccctgggacccctcaccgggctggctcccttcggccccttcccccaccccactccacagcacccagtgctggcaaaccgcgcgattccagcacgaaggaggaaacccaggagggtgcggcggcccgaggcgcacgcactcggccagcttccggcaactcaagggttacgaccaggcggcggcgcgcgccgaggggagaggcggtagctgacaggtggcgcctgcgcactgggagcgctcattgtgccccgcagctgccgaaccgcccgcccgccgcccgttcggagcgtcagtcggcgacagtctggtggtgggtgcggagtctgcggccgttcccgcggcctcctcctcctccccgttcccttcacccccaccccgcacccctttccccatcccggctccgtcaccctcccgtcccccacactcaggacaagaatgccctgcccggaacaacccagcagcgcctagatggctttggtcacggtccagcggtcacctacccccagcaccacctccagcccctgcgcctcggaggcagacagtggggaggaagaatgccggtcacagcccaggagcatcagcgagagctttctaactgtcaaaggtgctgccctttttctaccacggggaaatggctcatccacaccaagaatcagccacagacggaacaagcatgcaggcgatctccaacagcatctccaagcaatgttcattttactccgcccagaagacaacatcaggctggctgtaagactggaaagtacttaccagaatcgaacacgctatatggtagtggtttcaactaatggtagacaagacactgaagaaagcatcgtcctaggaatggatttctcctctaatgacagtagcacttgtaccatgggcttagttttgcctctctggagcgacacgctaattcatttggatggtgatggtgggttcagtgtatcgacggataacagagttcacatattcaaacctgtatctgtgcaggcaatgtggtctgcactacagagcttacacaaggcttgtgaagtcgccagagcgcataactactacccaggcagcctatttctcacttgggtgagttattatgagagccatatcaactcagatcaatcctcagtcaatgaatggaatgcaatgcaagatgtacagtcccaccggcccgactctccagctctcttcaccgacatacctactgaacgtgaacgaacagaaaggctaattaaaaccaaattaagggagatcatgatgcagaaggatttggagaatattacatccaaagagataagaacagagttggaaatgcaaatggtgtgcaacttgcgggaattcaaggaatttatagacaatgaaatgatagtgatccttggtcaaatggatagccctacacagatatttgagcatgtgttcctgggctcagaatggaatgcctccaacttagaggacttacagaaccgaggggtacggtatatcttgaatgtcactcgagagatagataacttcttcccaggagtctttgagtatcataacattcgggtatatgatgaagaggcaacggatctcctggcgtactggaatgacacttacaaattcatctctaaagcaaagaaacatggatctaaatgccttgtgcactgcaaaatgggggtgagtcgctcagcctccaccgtgattgcctatgcaatgaaggaatatgaccgagcctatgactatgtgaaagaaagacgaacggtaaccaagcccaacccaagcttcatgagacaactggaagagtatcaggggatcttgctggcaagcttcctaggcttgattcatggagggagggacaagccctggggagagaaaagcacagaatttgagtcagtagatctggtttccattcctggttcaccctcttgctgcaaccctgagaagttacttcacatttctcatccttacctgaccccatctataaaatgaaaatcaagagatccatctcacagggttattgtgaataaaaatgtgtttgaatgtt

SEQ ID NO: 4 - DNA, 4360 bp, Homo sapiens
gaagaagccctgaggaaggaggtggctggttcctgtctcactggccggagcttcagcttcagtcatttcatctgggtccctcagcccttggtggggaacatccaggcaggttagaggtgagtgtgcttctccccttgtcaatcctggtgagcccaggacttacaatacaaggggcagcggctttgccctgagtgtcagggcacaaccatgatggctgggaccagctgctggtatccttcatgtcctttaataggctccaggatgacgcctgagccaaaggccctacctcctgtggccttggttagagacaccgaaggccagctgtgtcttccccagcagagacaaagaggttggcaggttgtcatggcgaccagaaaggacacagaggaggagcaggtagtcccaagcgaggaggacgaagccaacgtgagggcggtgcaggcccactacctccgaagcccctcccctagccagtattcgatggtctcagatgcagaaacagaaagcattttcatggaacccattcacctctcctcagccattgcagccaaacagatcatcaatgaagaactcaagccaccgggggtcagagcagacgcagagtgtccaggcatgctggagtctgctgaacagctgctggtggaggacctgtacaaccgcgtcagggagaagatggatgacaccagcctctataatacgccctgtgtcctggacctacagcgggccctggttcaggatcgccaagaggcgccctggaatgaggtggatgaggtctggcccaatgtcttcatagctgagaagagtgtggctgtgaacaaggggaggctgaagaggctgggaatcacccacattctgaatgctgcgcatggcaccggcgtttacactggccccgaattctacactggcctggagatccagtacctgggtgtagaggtggatgactttcctgaggtggacatttcccagcatttccggaaggcgtactgtcattacatcattttctcttgtgttttcatttcagggaaagtcctggtcagcagcgaaatgggcatcagccggtcagcagtgctggtggtcgcctacctgatgatcttccacaacatggccatcctggaggctttgatgaccgtgcgtaagaagcgggccatctaccccaatgacggcttcctgaagcagctgcgggagctcaatgagaagttgatggaggagagagaagaggactatggccgggaggggggatcagctgaggctgaggagggcgagggcactgggagcatgctcggggccagagtgcacgccctgacggtggaagaggaggacgacagcgccagccacctgagtggctcctccctggggaaggccacccaggcctccaagcccctcaccctcatagacgaggaggaggaggagaaactgtacgagcagtggaagaaggggcagggcctcctctcagacaaggtcccccaggatggaggtggctggcgctcagcctcctctggccagggtggggaggagctcgaggacgaggacgtggagaggatcatccaggagtggcagagccgaaacgagaggtaccaagcagaagggtaccggaggtggggaagggaggaggagaaggaggaggagagcgacgctggctcctcggtggggaggcggcggcgcaccctgagcgagagcagcgcctgggagagcgtgagcagccacgacatctgggtcctgaagcagcagctggagctgaaccgcccggaccacggcaggaggcgccgcgcagactcgatgtcctcggagagcacctgggacgcatggaacgagaggctgctggagattgagaaggaggcttcccggaggtaccacgccaagagcaagagagaggaggcggcagacaggagctcagaagcagggagcagggtgcgggaggatgatgaggacagcgtgggctctgaggccagttccttctacaacttctgcagcaggaacaaggacaagctcactgccctggaaagatggaagatcaagagaatccaatttggatttcacaagaaagacttgggagcgggagacagcagcggtgagcccggtgcagaggaggcagtaggggagaagaacccctccgacgtcagcctgacagcctaccaggcctggaagctgaaacaccagaagaaggtgggcagtgagaacaaggaggaggtggtggagctcagcaagggggaggactcggccttggctaagaagagacaacggaggctggagctgctggagagaagccggcagacgctggaggagagccagtctatggcaagctgggaggcggacagctccacggccagcgggagcattcccctgtctgcgttctggtctgcagacccctcagtcagcgctgatggggacacgacgtcagtactgagcacccagagccaccgctcccacctgtctcaggctgcaagcaacatagcggggtgttcaacctccaaccccaccacacccctgcctaacctgccagtggggcctggagacaccatttccattgccagtatccagaactggattgccaatgtagtcagtgagacccttgctcagaagcaaaatgaaatgctgctgttgtcccgctcaccgtctgttgcaagcatgaaggcagtaccagcggctagctgcctgggggatgaccaagtctccatgcttagtggacacagcagctcctccttgggtggctgcctgttgcctcagagccaggcaagacccagctctgacatgcagtctgtgctgtcctgcaacaccacactgagctcacccgcggaaagttgcagaagcaaagtgagggggaccagcaagcccatcttcagcctctttgctgacaatgtggacctaaaggaacttggccggaaggagaaggagatgcagatggagcttagggagaagatgtctgagtaccaaatggaaaagctggcctcagacaacaaacgcagctccctcttcaagaagaagaaggtcaaggaagatgaggatgatggtgtgggtgatggggatgaggacactgacagtgccatagggagcttccgatattcttcccgcagtaattcccagaaacctgaaacagacacatgctcctccctggctgtctgtgatcactatgcaagtggcagcagagttggcaaagagatggatagcagtattaataagtggctcagtggcctcaggacggaggaaaaacctcctttccaaagtgactggtctggaagttccagagggaagtacaccagatcgtccctgctcagggagacagagtctaaatcctccagttacaagttttccaaatcccagtcagaggaacaggtacacctcctcctaccacgaggcaaatggcaactctgtaagaagcacttcacggttctcatcttcctccaccagggagggcagagagatgcacaagttctccaggtccacgtacaacgagacctcaagttcccgagaggagagcccagagccctacttcttccgccggaccccagagtcctcagaaagggaagagtccccagaaccacagcgcccaaattgggccaggtccagggactgggaagatgtggaagagtcatccaagtcagacttctctgaatttggagccaagaggaagttcacccagagctttatgaggtctgaagaagagggagagaaagagaggacagaaaacagagaagaagggaggtttgcatctggacggcggtcccagtatcggagaagcaatgacagggaggaagaggaagaaatggacgatgaagccatcattgctgcttggagacgccggcaagaagaaaccaggaccaagctgcagaaaaggagggaggactgagctggggaaaatctgagaacactgaaagaaaccactcacgttagcatagggctcagggcacacgttgccaccactcatcgcaggatgaggatacagagaggatcttccagaggggcagagccaaaatgagaggtaccaagcataagggcagcagaggtggagtagggaggaggcaaggagggggagaaccatcaatacgaatacgaggtccgaatgctggaccaactgataccattttctgttgctcagcgccctctaagctttggtgtttcacttaatgtatttgacagtgttcatcacaggctagagaggtgagcttggaaaagcactgtagtttgtcagagactccagtttacatccagaaaggccatgaacataggacacgcttctgtctgtagaggcttcatatgagacccagaaagtctatcctatggcaagtctgacctctcctggcaatgctcagttctgattttttttttttaatgttttgagtctccattaaaaatggtatgttgg

SEQ ID NO: 5 - DNA, 1262 bp, Homo sapiens
gtcaagggtttcaggtcgcactggaaaatcattttgcaagcagatgtcataggtctcctcttagactggacggcacgcaaggtcagcgtcacagatctgaccctaaaaataggcctctgttgccagtcggggtggctgggcgtgcggctgctacatgccccacggaccagaacctcccgacgcggccaggccccggcacacccagctgcagaaaggagagaaaatcccttggctctaaaatgacatctggagaagtgaagacaagcctcaagaatgcctactcatctgccaagaggctgtcgccgaagatggaggaggaaggggaggaggaggactactgcacccctggagcctttgagctggagcggctcttctggaagggcagtccccagtacacccacgtcaacgaggtctggcccaagctctacattggcgatgaggcgacggcgctggaccgctataggctgcagaaggcggggttcacgcacgtgctgaacgcggcccacggccgctggaacgtggacactgggcccgactactaccgcgacatggacatccagtaccacggcgtggaggccgacgacctgcccaccttcgacctcagtgtcttcttctacccggcggcagccttcatcgacagagcgctaagcgacgaccacagtaagatcctggttcactgcgtcatgggccgcagccggtcagccaccctggtcctggcctacctgatgatccacaaggacatgaccctggtggacgccatccagcaagtggccaagaaccgctgcgtcctcccgaaccggggctttttgaagcagctccgggagctggacaagcagctggtgcagcagaggcgacggtcccagcgccaggacggtgaggaggaggatggcagggagctgtaggcccgactcacagggccagcagaggcacttggggacagaggggagaggcagaacatagccctggcctaggactccagagaagggatggtgaaaccgaagctcgactcttccaaaccatcttgttcaacttccccatgtgtgctggggacagggaggacccagagctgcccccgggcagagctgagcgctcagcctctcagcaaaatgggagggacgggctccccggctctgggtcacagaggagcatgccacgctgcaccaagtctcctgctttggttttgtttttttggtgagaaggaagagggaaaagattttaaaatgtggggctttatttttgtaaatatccttcgggctttgttt

SEQ ID NO: 6 - DNA, 1917 bp, Homo sapiens
acagaggcagaggggtgggcgggctggcccatggctgagacctctctcccagagctggggggagaggacaaagccacgccttgccccagcatcctggagctggaggagctcctgcgggcagggaagtcttcttgcagccgtgtggacgaagtttggcccaaccttttcataggagatgcggccacggcaaacaaccgctttgagctgtggaagctgggcatcacccacgtgctgaacgccgcccacaagggcctctactgtcagggcggccctgacttctacggcagcagtgtgagctacctgggggtgccagcccacgacctccctgattttgacatcagtgcctacttctcctctgcggctgacttcatccaccgtgccctcaacacgcctggggccaaggtcctggtgcactgtgtggtgggcgtgagccgctctgccacgctggtcctggcctacctcatgctgcaccagcggctgtccctgcgccaggcggtgatcaccgtgaggcagcaccgatgggtcttccccaaccgaggcttcctgcaccagctctgcaggctggaccactggtccttactccctgccatggggctctgccactttgccaccctggcactgatcctgctggtgctgctggaggctctggcccaggcggacacacagaagatggtggaagcccagcgtggggtcggccctagagcctgctactccatctggctcctcctggcgcctacaccccctctcagccactgtcttcagtctccacagaaacagcatcaagtgtgcggagacaggcggctgaaagccagcagcacgaactgcccgtcagagaagtgcacagcctgggccagatactcccacaggtgggcccatattctggtgccgctgaaaatccagctccgcagggtccctgactccttcagccagcagatgcctgaaacaagctacctgacccgggtggggcctgacatccagtgctggcctgagtcgtgggggatggactcactgcagaagcaggacctccggaggcccaagatccatggggcagtccaggcatctccctaccagccgcccacattggcttcgctgcagcgcttgctgtgggtccgtcaggctgccacactgaaccatatcgatgaggtctggcccagcctcttcctgggagatgcgtacgcagcccgggacaagagcaagctgatccagctgggaatcacccacgttgtgaatgccgctgcaggcaagttccaggtggacacaggtgccaaattctaccgtggaatgtccctggagtactatggcatcgaggcggacgacaaccccttcttcgacctcagtgtctactttctgcctgttgctcgatacatccgagctgccctcagtgttccccaagaggatggccatgggtgtctcttcttcccaaaggggtgggtggttcaagggcaggtagctgatgctaagttggttctccctacaggccgcgtgctggtacactgtgccatgggggtaagccgctctgccacacttgtcctggccttcctcatgatctgtgagaacatgacgctggtagaggccatccagacggtgcaggcccaccgcaatatctgccctaactcaggcttcctccggcagctccaggttctggacaaccgactggggcgggagacggggcggttctgatctggcaggcagccaggatccctgacccttggcccaaccccaccagcctggccctgggaacagcaggctctgctgtttctagtgaccctgagatgtaaacagcaagtgggggctgaggcagaggcagggatagctgggtggtgacctcttagcgggtggatttccctgacccaattcagagattctttatgcaaaagtgagttcagtccatctctataataaaatattcatcgtcat

SEQ ID NO: 7 - DNA, 636 bp, Homo sapiens
atgtgccctggtaactggctttgggcttctatgacttttatggcccgcttctcccggagtagctcaaggtctcctgttcgaactcgagggaccctggaggagatgccaaccgttcaacatcctttcctcaatgtcttcgagttggagcggctcctctacacaggcaagacagcctgtaaccatgccgacgaggtctggccaggcctctatctcggagaccaggacatggctaacaaccgccgggagcttcgccgcctgggcatcacgcacgtcctcaatgcctcacacagccggtggcgaggcacgcccgaggcctatgaggggctgggcatccgctacctgggtgttgaggcccacgactcgccagcctttgacatgagcatccacttccagacggctgccgacttcatccaccgggcgctgagccagccaggagggaagatcctggtgcattgtgctgtgggcgtgagccgatccgccaccctggtactggcctacctcatgctgtaccaccaccttaccctcgtggaggccatcaagaaagtcaaagaccaccgaggcatcatccccaaccggggcttcctgaggcagctcctggccctggaccgcaggctgcggcagggtctggaagcatga

SEQ ID NO: 8 - DNA, 1326 bp, Homo sapiens
atgcaggggcagactgtagttccaaaagattcctacactatatcccttatccagaggctgcggggccgtgaggccgcaaggagaacccatgagaaccttcttcggctgtctgccctagtgagatccccacagacagctagcatcgactgccacacgtggtcagtttctagtggaaccaatacttcgctgcaggcgtcgggcctgggccgtcagggcagctgtgaccggatcgcttcccgggcggcgagctgggggtgcacccggaccgccgcccccgggatcatgggcaatggcatgaccaaggtacttcctggactctacctcggaaacttcattgatgccaaagacctggatcagctgggccgaaataagatcacacacatcatctctatccatgagtcaccccagcctctgctgcaggatatcacctaccttcgcatcccggtcgctgatacccctgaggtacccatcaaaaagcacttcaaagaatgtatcaacttcatccactgctgccgccttaatggggggaactgccttgtgcactgctttgcaggcatctctcgcagcaccacgattgtgacagcgtatgtgatgactgtgacggggctaggctggcgggacgtgcttgaagccatcaaggccaccaggcccatcgccaaccccaacccaggctttaggcagcagcttgaagagtttggctgggccagttcccagaagcttcgccggcagctggaggagcgcttcggcgagagccccttccgcgacgaggaggagttgcgcgcgctgctgccgctgtgcaagcgctgccggcagggctccgcgacctcggcctcctccgccgggccgcactcagcagcctccgagggaaccgtgcagcgcctggtgccgcgcacgccccgggaagcccaccggccgctgccgctgctggcgcgcgtcaagcagactttctcttgcctcccccggtgtctgtcccgcaagggcggcaagtgaggatgcagtccagccgtggctccccacttccgactggctcccttcgggggctgtctgcgccttccacgccccccaggacgggcccagaggctgggggagccccgcggcggcctgaaccctgcctcccgcgcccgccctgctcgtccgcgtctgcagtcagcgtccccaacctgtgcgtctctgtgtccgggccggcctgctgcagccacctggtgccttagtccttgggctgggggagggggcccacccttaaaggcggcgggaggggagggagggagagtggagggtttgacgggcctggagggtattaaagagacacagaagaagctgcctgtc

SEQ ID NO: 9 - DNA, 1083 bp, Homo sapiens
atgatagaggatacaatgactttgctgtctctgctgggtcgcatcatgcgctacttcttgctgagacccgagacgcttttcctgctgtgcatcagcttggctctatggagttacttcttccacaccgacgaggtgaagaccatcgtgaagtccagccgggacgccgtgaagatggtgaagagcaaggtagccgagaccatgcagaacgatcgactcggggggcttgatgtgctcgaggccgagttttccaagacctgggagttcaagaaccacaacgtggcggtgtactccatccagggccggagagaccacatggaggaccgcttcgaagttctcacggatctggccaacaagacgcacccgtccatcttcgggatcttcgacgggcacgggggagagactgcagctgaatatgtaaaatctcgactcccagaggctcttaaacagcatcttcaggactacgagaaagacaaagaaaatagtgtattatcttaccagaccatccttgaacagcagattttgtcaattgaccgagaaatgctagaaaaattgactgtatcctatgatgaagcaggcacaacgtgtttgattgctctgctatcagataaagacctcactgtggccaacgtgggtgactcgcgcggggtcctgtgtgacaaagatgggaacgctattcctttgtctcatgatcacaagccttaccagttgaaggaaagaaagaggataaagagagcaggtggtttcatcagtttcaatggctcctggagggtccagggaatcctggccatgtctcggtccctgggggattatccgctgaaaaatctcaacgtggtcatcccagacccagacatcctgacctttgacctggacaagcttcagcctgagttcatgatcttggcatcagatggtctctgggatgctttcagcaatgaagaagcagttcgattcatcaaggagcgcttggatgaacctcactttggggccaagagcatagttttacagtcattttacagaggctgccctgacaatataacagtcatggtggtgaagttcagaaatagcagcaaaacagaagagcagtga

SEQ ID NO: 10 - DNA, 1725 bp, Homo sapiens
atgttgtcggctccgtgttgtgatgacaggagaatgtgtgtgtgtcccgggcccagacgaattggaatcccagtcagaagttccagcctgccactgttctctgatgccatgccagcaccaactcaactgttttttcctctcatccgtaactgtgaactgagcaggatctatggcactgcatgttactgccaccacaaacatctctgttgttcctcatcgtacattcctcagagtcgactgagatacacacctcatccagcatatgctaccttttgcaggccaaaggagaactggtggcagtacacccaaggaaggagatatgcttccacaccacagaaattttacctcacacctccacaagtcaatagcatccttaaagctaatgaatacagtttcaaagtgccagaatttgacggcaaaatgtcagttctatccttggatttgacagcaatcaagctgcctgcaaatgcacccattgaggaccggagaagtgcagcaacctgcttgcagaccagagggatgcttttgggggtttttgatggccatgcaggttgtgcttggtcccaggcagtcagtgaaagactcttttattatattgctggctctttggtaccccatgagactttgctagagattgaaaatgcagtggagagcggccgggcactgctacccattctccagtggcacaagcaccccaatgattactttagtaaggaggcatccaaattgtactttaacagcttgaggacttactggcaagagcttatagacctcaacactggtgagtcgactgatattgatgttaaggaggctctaattaatgccttcaagaggcttgataatgacatctccttggaggcgcaagttggtgatcctaattcttttctcaactacctggtgcttcgagtggcattttctggagccactgcttgtgtggcccatgtggatggtgttgaccttcatgtggccaatactggcgatagcagagccatgctgggtgtgcaggaagaggacggctcatggtcagcagtcacgctgtctaatgaccacaatgctcaaaatgaaagagaactagaacggctgaaattggaacatccaaagagtgaggccaagagtgtcgtgaaacaggatcggctgcttggcttgctgatgccatttagggcatttggagatgtaaagttcaaatggagcattgaccttcaaaagagagtgatagaatctggcccagaccagttgaatgacaatgaatataccaagtttattcctcctaattatcacacacctccttatctcactgctgagccagaggtaacttaccaccgattaaggccacaggataagtttctggtgttggctactgatgggttgtgggagactatgcataggcaggatgtggttaggattgtgggtgagtacctaactggcatgcatcaccaacagccaatagctgttggtggctacaaggtgactctgggacagatgcatggccttttaacagaaaggagaaccaaaatgtcctcggtatttgaggatcagaacgcagcaacccatctcattcgccacgctgtgggcaacaacgagtttgggactgttgatcatgagcgcctctctaaaatgcttagtcttcctgaagagcttgctcgaatgtacagagatgacattacaatcattgtagttcagttcaattctcatgttgtaggggcgtatcaaaaccaagaaaagtga

SEQ ID NO: 11 - DNA, 909 bp, Homo sapiens
atgaggctcccaatcctgttcgctgccctgctctggttccggggttttctggcagaggaggaagcatgcctctccctggaagggagtccaggcagggagagtgcaggcccacccgtgaacgtgaacatcaccagccaggggagacctactagcctctttctgagctgggcagccccggggccaggcaggttcacccatgccctccgcctcacatgtctgagccccctcagctctcctgaagggcagcagctccaggcccacaccaatgcatccagctttaagttccaagatctggtgtcagggggtcgctaccagctggaagtgactgccctgcgaccctgtgggcagaatgtcaccatcaccctcactgctcgcactgccccgtcaactgtccatggactgcagctccactctgggagcccatccagcctggaggcctcatggggtgatgcccccgggaagcaggatggctactgccttctcctctaccacctagaatcccagacattggcacataatatctccatgcccctgggcaccctgtcctacaattttggcaacctcttgccaggtattgagtatattttggaagttaacacctgggctggcaacctccaagcaacaaccagcctccatcagtggacagcccctgtgtctccagatcacctggttctgcataccctgggcaccagtgccttgcaagcctcctggaacggctccaagggggctgcctggctccacttggtgctcacagacctacctggtggcaccaatctgactgcagtattcagacggggagtctcccatcacacctcccttcacctgtctcagggccccccctatgagctgacgctcagtgctgctgccaggccccatcgggcagtggggcccaatgccacagagtggacc

SEQ ID NO: 12 - DNA, 354 bp, Homo sapiens
  modified_base (1)..(354): “n” represents a, t, c , g, other or unknown
tctgaaggagttggctggactggttgtttcattgtcatagatgccatgttggaaagaatcaagcatgaaaaaactgtaggtaattatgcctatgcaactttaatgagaacccagaggaattacatggttcaagcaggagaccagtgtatctctgtccatgatgcactgttagaggcagttacttgtgtaaataccaaagttccagctagaaacttgtatgcctatattangaaactgacacaaatagagaggggacagaatgtcataggagtggtgctcaaatttaagcatctaatcagctcaaaagctcacatctcaggtttcctcagtgccaatcttccatgcaataatttc

SEQ ID NO: 13 - protein, 1049 aa, Homo sapiens
MALVTLQRSPTPSAASSSASNSELEAGSEEDRKLNLSLSESFFMVKGAALFLQQGSSPQGQRSLQHPHKHAGDLPQHLQVMINLLRCEDRIKLAVRLESAWADRVRYMVVVYSSGRQDTEENILLGVDFSSKESKSCTIGMVLRLWSDTKIHLDGDGGFSVSTAGRMHIFKPVSVQAMWSALQVLHKACEVARRHNYFPGGVALIWATYYESCISSEQSCINEWNAMQDLESTRPDSPALFVDKPTEGERTERLIKAKLRSIMMSQDLENVTSKEIRNELEKQMNCNLKELKEFIDNEMLLILGQMDKPSLIFDHLYLGSEWNASNLEELQGSGVDYILNVTREIDNFFPGLFAYHNIRVYDEETTDLLAHWNEAYHFINKAKRNHSKCLVHCKMGVSRSASTVIAYAMKEFGWPLEKAYNYVKQKRSITRPNAGFMRQLSEYEGILDASKQRHNKLWRQQTDSSLQQPVDDPAGPGDFLPETPDGTPESQLPFLDDAAQPGLGPPLPCCFRRLSDPLLPSPEDETGSLVHLEDPEREALLEEAAPPAEVHRPARQPQQGSGLCEKDVKKKLEFGSPKGRSGSLLQVEETEREEGLGAGRWGQLPTQLDQNLLNSENLNNNSKRSCPNGMEDDAIFGILNKVKPSYKSCADCMYPTASGAPEASRERCEDPNAPAICTQPAFLPHITSSPVAHLASRSRVPEKPASGPTEPPPFLPPAGSRRADTSGPGAGAALEPPASLLEPSRETPKVLPKSLLLKNSHCDKNPPSTEVVIKEESSPKKDMKPAKDLRLLFSNESEKPTTNSYLMQHQESIIQLQKAGLVRKHTKELERLKSVPADPAPPSRDGPASRLEASIPEESQDPAALHELGPLVMPSQAGSDEKSEAAPASLEGGSLKSPPPFFYRLDHTSSFSKDFLKTICYTPTSSSMSSNLTRSSSSDSIHSVRGKPGLVKQRTQEIETRLRLAGLTVSSPLKRSHSLAKLGSLTFSTEDLSSEADPSTVADSQDTTLSESSFLHEPQGTPRDPAATSKPSGKPAPENLKSPSWMSKS

SEQ ID NO: 14 - protein, 665 aa, Homo sapiens
MAHEMIGTQIVTERLVALLESGTEKVLLIDSRPFVEYNTSHILEAININCSKLMKRRLQQDKVLITELIQHSAKHKVDIDCSQKVVVYDQSSQDVASLSSDCFLTVLLGKLEKSFNSVHLLAGGFAEFSRCFPGLCEGKSTLVPTCISQPCLPVANIGPTRILPNLYLGCQRDVLNKELMQQNGIGYVLNASNTCPKPDFIPESHFLRVPVNDSFCEKILPWLDKSVDFIEKAKASNGCVLVHCLAGISRSATIAIAYIMKRMDMSLDEAYRFVKEKRPTISPNFNFLGQLLDYEKKIKNQTGASGPKSKLKLLHLEKPNEPVPAVSEGGQKSETPLSPPCADSATSEAAGQRPVHPASVPSVPSVQPSLLEDSPLVQALSGLHLSADRLEDSNKLKRSFSLDIKSVSYSASMAASLHGFSSSEDALEYYKPSTTLDGTNKLCQFSPVQELSEQTPETSPDKEEASIPKKLQTARPSDSQSKRLHSVRTSSSGTAQRSLLSPLHRSGSVEDNYHTSFLFGLSTSQQHLTKSAGLGLKGWHSDILAPQTSTPSLTSSWYFATESSHFYSASAIYGGSASYSAYSCSQLPTCGDQVYSVRRRQKPSDRADSRRSWHEESPFEKQFKRRSCQMEFGESIMSENRSREELGKVGSQSSFSGSMEIIEVS

SEQ ID NO: 15 - protein, 498 aa, Homo sapiens
MALVTVQRSPTPSTTSSPCASEADSGEEECRSQPRSISESFLTVKGAALFLPRGNGSSTPRISHRRNKHAGDLQQHLQAMFILLRPEDNIRLAVRLESTYQNRTRYMVVVSTNGRQDTEESIVLGMDFSSNDSSTCTMGLVLPLWSDTLIHLDGDGGFSVSTDNRVHIFKPVSVQAMWSALQSLHKACEVARAHNYYPGSLFLTWVSYYESHINSDQSSVNEWNAMQDVQSHRPDSPALFTDIPTERERTERLIKTKLREIMMQKDLENITSKEIRTELEMQMVCNLREFKEFIDNEMIVILGQMDSPTQIFEHVFLGSEWNASNLEDLQNRGVRYILNVTREIDNFFPGVFEYHNIRVYDEEATDLLAYWNDTYKFISKAKKHGSKCLVHCKMGVSRSASTVIAYAMKEYDRAYDYVKERRTVTKPNPSFMRQLEEYQGILLASFLGLIHGGRDKPWGEKSTEFESVDLVSIPGSPSCCNPEKLLHISHPYLTPSIK

SEQ ID NO: 16 - protein, 1133 aa, Homo sapiens
MMAGTSCWYPSCPLIGSRMTPEPKALPPVALVRDTEGQLCLPQQRQRGWQVVMATRKDTEEEQVVPSEEDEANVRAVQAHYLRSPSPSQYSMVSDAETESIFMEPIHLSSAIAAKQIINEELKPPGVRADAECPGMLESAEQLLVEDLYNRVREKMDDTSLYNTPCVLDLQRALVQDRQEAPWNEVDEVWPNVFIAEKSVAVNKGRLKRLGITHILNAAHGTGVYTGPEFYTGLEIQYLGVEVDDFPEVDISQHFRKAYCHYIIFSCVFISGKVLVSSEMGISRSAVLVVAYLMIFHNMAILEALMTVRKKRAIYPNDGFLKQLRELNEKLMEEREEDYGREGGSAEAEEGEGTGSMLGARVHALTVEEEDDSASHLSGSSLGKATQASKPLTLIDEEEEEKLYEQWKKGQGLLSDKVPQDGGGWRSASSGQGGEELEDEDVERIIQEWQSRNERYQAEGYRRWGREEEKEEESDAGSSVGRRRRTLSESSAWESVSSHDIWVLKQQLELNRPDHGRRRRADSMSSESTWDAWNERLLEIEKEASRRYHAKSKREEAADRSSEAGSRVREDDEDSVGSEASSFYNFCSRNKDKLTALERWKIKRIQFGFHKKDLGAGDSSGEPGAEEAVGEKNPSDVSLTAYQAWKLKHQKKVGSENKEEVVELSKGEDSALAKKRQRRLELLERSRQTLEESQSMASWEADSSTASGSIPLSAFWSADPSVSADGDTTSVLSTQSHRSHLSQAASNIAGCSTSNPTTPLPNLPVGPGDTISIASIQNWIANVVSETLAQKQNEMLLLSRSPSVASMKAVPAASCLGDDQVSMLSGHSSSSLGGCLLPQSQARPSSDMQSVLSCNTTLSSPAESCRSKVRGTSKPIFSLFADNVDLKELGRKEKEMQMELREKMSEYQMEKLASDNKRSSLFKKKKVKEDEDDGVGDGDEDTDSAIGSFRYSSRSNSQKPETDTCSSLAVCDHYASGSRVGKEMDSSINKWLSGLRTEEKPPFQSDWSGSSRGKYTRSSLLRETESKSSSYKFSKSQSEEQVHLLLPRGKWQLCKKHFTVLIFLHQGGQRDAQVLQVHVQRDLKFPRGEPRALLLPPDPRVLRKGRVPRTTAPKLGQVQGLGRCGRVIQVRLL

SEQ ID NO: 17 - protein, 220 aa, Homo sapiens
MTSGEVKTSLKNAYSSAKRLSPKMEEEGEEEDYCTPGAFELERLFWKGSPQYTHVNEVWPKLYIGDEATALDRYRLQKAGFTHVLNAAHGRWNVDTGPDYYRDMDIQYHGVEADDLPTFDLSVFFYPAAAFIDRALSDDHSKILVHCVMGRSRSATLVLAYLMIHKDMTLVDAIQQVAKNRCVLPNRGFLKQLRELDKQLVQQRRRSQRQDGEEEDGREL

SEQ ID NO: 18 - protein, 549 aa, Homo sapiens [sequence omitted]

SEQ ID NO: 19 - protein, 211 aa, Homo sapiens
MCPGNWLWASMTFMARFSRSSSRSPVRTRGTLEEMPTVQHPFLNVFELERLLYTGKTACNHADEVWPGLYLGDQDMANNRRELRRLGITHVLNASHSRWRGTPEAYEGLGIRYLGVEAHDSPAFDMSIHFQTAADFIHRALSQPGGKILVHCAVGVSRSATLVLAYLMLYHHLTLVEAIKKVKDHRGIIPNRGFLRQLLALDRRLRQGLEA

SEQ ID NO: 20 - protein, 329 aa, Homo sapiens
MQGQTVVPKDSYTISLIQRLRGREAARRTHENLLRLSALVRSPQTASIDCHTWSVSSGTNTSLQASGLGRQGSCDRIASRAASWGCTRTAAPGIMGNGMTKVLPGLYLGNFIDAKDLDQLGRNKITHIISIHESPQPLLQDITYLRIPVADTPEVPIKKHFKECINFIHCCRLNGGNCLVHCFAGISRSTTIVTAYVMTVTGLGWRDVLEAIKATRPIANPNPGFRQQLEEFGWASSQKLRRQLEERFGESPFRDEEELRALLPLCKRCRQGSATSASSAGPHSAASEGTVQRLVPRTPREAHRPLPLLARVKQTFSCLPRCLSRKGGK

SEQ ID NO: 21 - protein, 360 aa, Homo sapiens
MIEDTMTLLSLLGRIMRYFLLRPETLFLLCISLALWSYFFHTDEVKTIVKSSRDAVKMVKSKVAETMQNDRLGGLDVLEAEFSKTWEFKNHNVAVYSIQGRRDHMEDRFEVLTDLANKTHPSIFGIFDGHGGETAAEYVKSRLPEALKQHLQDYEKDKENSVLSYQTILEQQILSIDREMLEKLTVSYDEAGTTCLIALLSDKDLTVANVGDSRGVLCDKDGNAIPLSHDHKPYQLKERKRIKRAGGFISFNGSWRVQGILAMSRSLGDYPLKNLNVVIPDPDILTFDLDKLQPEFMILASDGLWDAFSNEEAVRFIKERLDEPHFGAKSIVLQSFYRGCPDNITVMVVKFRNSSKTEEQ

SEQ ID NO: 22 - protein, 574 aa, Homo sapiens
MLSAPCCDDRRMCVCPGPRRIGIPVRSSSLPLFSDAMPAPTQLFFPLIRNCELSRIYGTACYCHHKHLCCSSSYIPQSRLRYTPHPAYATFCRPKENWWQYTQGRRYASTPQKFYLTPPQVNSILKANEYSFKVPEFDGKMSVLSLDLTAIKLPANAPIEDRRSAATCLQTRGMLLGVFDGHAGCAWSQAVSERLFYYIAGSLVPHETLLEIENAVESGRALLPILQWHKHPNDYFSKEASKLYFNSLRTYWQELIDLNTGESTDIDVKEALINAFKRLDNDISLEAQVGDPNSFLNYLVLRVAFSGATACVAHVDGVDLHVANTGDSRAMLGVQEEDGSWSAVTLSNDHNAQNERELERLKLEHPKSEAKSVVKQDRLLGLLMPFRAFGDVKFKWSIDLQKRVIESGPDQLNDNEYTKFIPPNYHTPPYLTAEPEVTYHRLRPQDKFLVLATDGLWETMHRQDVVRIVGEYLTGMHHQQPIAVGGYKVTLGQMHGLLTERRTKMSSVFEDQNAATHLIRHAVGNNEFGTVDHERLSKMLSLPEELARMYRDDITIIVVQFNSHVVGAYQNQEK

SEQ ID NO: 23 - protein, 303 aa, Homo sapiens
MRLPILFAALLWFRGFLAEEEACLSLEGSPGRESAGPPVNVNITSQGRPTSLFLSWAAPGPGRFTHALRLTCLSPLSSPEGQQLQAHTNASSFKFQDLVSGGRYQLEVTALRPCGQNVTITLTARTAPSTVHGLQLHSGSPSSLEASWGDAPGKQDGYCLLLYHLESQTLAHNISMPLGTLSYNFGNLLPGIEYILEVNTWAGNLQATTSLHQWTAPVSPDHLVLHTLGTSALQASWNGSKGAAWLHLVLTDLLGGTNLTAVFRRGVSHHTSLHLSQGPPYELTLSAAARPHRAVGPNATEWT

SEQ ID NO: 24 - protein, 118 aa, Homo sapiens
  MOD_RES (1)..(118): “Xaa” represents any, other or unknown amino acid
SEGVGWTGCFIVIDAMLERIKHEKTVGNYAYATLMRTQRNYMVQAGDQCISVHDALLEAVTCVNTKVPARNLYAYIXKLTQIERGQNVIGVVLKFKHLISSKAHISGFLSANLPCNNF

SEQ ID NO: 25 - protein, 12 aa, Artificial Sequence
  Description of Artificial Sequence Synthetic peptide
SEFLDEALLTYR

SEQ ID NO: 26 - DNA, 34 bp, Artificial Sequence
  Description of Artificial Sequence Primer
ctgagttcctggatgaggcgctgctgacttacag

SEQ ID NO: 27 - protein, 163 aa, Artificial Sequence
  Description of Artificial Sequence Synthetic peptide
ANGNSVRSTSRFSSSSTREGREMHKFSRSTYNETSSSREESPEPYFFRRTPESSEREESPEPQRPNWARSRDWEDVEESSKSDFSEFGAKRKFTQSFMRSEEEGEKERTENREEGRFASGRRSQYRRSNDREEEEEMDDEAIIAAWRRRQEETRTKLQKRRED

SEQ ID NO: 28 - protein, 79 aa, Artificial Sequence [sequence omitted]

SEQ ID NO: 29 - protein, 26 aa, Artificial Sequence
  Description of Artificial Sequence Synthetic peptide
SGSSRFWTTDWGGRRGGSDLAGSQDP

SEQ ID NO: 30 - DNA, 57 bp, Artificial Sequence
  Description of Artificial Sequence Primer
aagcagtggtaacaacgcagagtacttttttttttttttttttttttttttttttvn

SEQ ID NO: 31 - DNA, 30 bp, Artificial Sequence
  Description of Artificial Sequence Primer
aagcagtggtaacaacgcagagtacgcggg

SEQ ID NO: 32 - DNA, 30 bp, Artificial Sequence
  Description of Artificial Sequence Primer
aagtggcaacagagataacgcgtacgcggg

SEQ ID NO: 33 - DNA, 23 bp, Artificial Sequence
  Description of Artificial Sequence Primer
aagcagtggtaacaacgcagagt

SEQ ID NO: 34 - DNA, 23 bp, Artificial Sequence
  Description of Artificial Sequence Primer
aagtggcaacagagataacgcgt

SEQ ID NO: 35 - DNA, 20 bp, Artificial Sequence
  Description of Artificial Sequence Primer
ggagctgtcgtattccagtc

SEQ ID NO: 36 - DNA, 21 bp, Artificial Sequence
  Description of Artificial Sequence Primer
aacccctcaagacccgtttag

SEQ ID NO: 37 - protein, 5 aa, Homo sapiens
MTPEK

SEQ ID NO: 38 - DNA, 18 bp, Homo sapiens
ccaaacataagtggcaca

SEQ ID NO: 39 - DNA, 17 bp, Homo sapiens
agaagatgtctgagtac

SEQ ID NO: 40 - DNA, 15 bp, Homo sapiens
catctaccccaatga

SEQ ID NO: 41 - DNA, 20 bp, Homo sapiens
cagcagcctccgagggaacc

SEQ ID NO: 42 - protein, 22 aa, Homo sapiens
GFSVSTAGRMHIFKPVSVQAMW

SEQ ID NO: 43 - DNA, 18 bp, Homo sapiens
ccaaacataagtggcaca

SEQ ID NO: 44 - protein, 13 aa, Homo sapiens
YCHYIIFSCVFIS

SEQ ID NO: 45 - DNA, 37 bp, Homo sapiens
actgtcattacatcattttctcttgtgttttcatttc

SEQ ID NO: 46 - protein, 4 aa, Homo sapiens
VHLL

SEQ ID NO: 47 - protein, 6 aa, Homo sapiens
MLESAE

SEQ ID NO: 48 - DNA, 17 bp, Homo sapiens
agaagatgtctgagtac

SEQ ID NO: 49 - DNA, 15 bp, Homo sapiens
catctaccccaatga

SEQ ID NO: 50 - DNA, 240 bp, Homo sapiens
caagggtttcaggtcgcactggaaaatcattttgcaagcagatgtcataggtctcctcttagactggacggcacgcaaggtcagcgtcacagatctgaccctaaaaataggcctctgttgccagtcggggtggctgggcgtgcggctgctacatgccccacggaccagaacctcccgacgcggccaggccccggcacacccagctgcagaaaggagagaaaatcccttggctctaaaatg

SEQ ID NO: 51 - protein, 50 aa, Homo sapiens
MAETSLPELGGEDKATPCPSILELEELLRAGKSSCSRVDEVWPNLFIGDA

SEQ ID NO: 52 - protein, 73 aa, Homo sapiens
ATANNRFELWKLGITHVLNAAHKGLYCQGGPDFYGSSVSYLGVPAHDLPDFDISAYFSSAADFIHRALNTPGA

SEQ ID NO: 53 - protein, 57 aa, Homo sapiens
KVLVHCVVGVSRSATLVLAYLMLHQRLSLRQAVITVRQHRWVFPNRGFLHQLCRLDH

SEQ ID NO: 54 - protein, 67 aa, Homo sapiens
WSLLPAMGLCHFATLALILLVLLEALAQADTQKMVEAQRGVGPRACYSIWLLLAPTPPLSHCLQSPQ

SEQ ID NO: 55 - protein, 33 aa, Homo sapiens
KQHQVCGDRRLKASSTNCPSEKCTAWARYSHRW

SEQ ID NO: 56 - protein, 43 aa, Homo sapiens
AHILVPLKIQLRRVPDSFSQQMPETSYLTRVGPDIQCWPESWG

SEQ ID NO: 57 - protein, 58 aa, Homo sapiens [sequence omitted]

SEQ ID NO: 58 - protein, 74 aa, Homo sapiens
YAARDKSKLIQLGITHVVNAAAGKFQVDTGAKFYRGMSLEYYGIEADDNPFFDLSVYFLPVARYIRAALSVPQE

SEQ ID NO: 59 - protein, 94 aa, Homo sapiens
DGHGCLFFPKGWVVQGQVADAKLVLPTGRVLVHCAMGVSRSATLVLAFLMICENMTLVEAIQTVQAHRNICPNSGFLRQLQVLDNRLGRETGRF

SEQ ID NO: 60 - protein, 4 aa, Homo sapiens
NSGF

SEQ ID NO: 61 - protein, 4 aa, Homo sapiens
MGNG

SEQ ID NO: 62 - DNA, 20 bp, Homo sapiens
cagcagcctccgagggaacc

SEQ ID NO: 63 - DNA, 19 bp, Homo sapiens
ccaaacataagtggcacar

SEQ ID NO: 64 - DNA, 16 bp, Homo sapiens
catctaccccaatgas

SEQ ID NO: 65 - DNA, 18 bp, Homo sapiens
agaagatgtctgagtacm

SEQ ID NO: 66 - DNA, 21 bp, Homo sapiens
cagcagcctccgagggaaccs

SEQ ID NO: 67 - DNA, 522 bp, Homo sapiens
gattccgcagccaagtccagacaaggcagtggtgccaagcggcagctggatgggctggaggcctccaaggagcccgggagacgggccctgctctacacagagggaaacccgggcctccttggaaacatctctgtgccacctggtgccacccacatcaccttctatgggccagtgcctggggcccgctactgtgtggacattgcctcatctctgggaatcatcacttacagcctcatgggccacaaaagtcccctggcaccacagtccctggaggttatcagcaggggtggcccctctgacctggccattgtctgggccccagcaccaggacagcgggaaggctacagggtcgcttggcaccaggagggcagccagaggtcaccgggcagtgcttgttgattgggcccggacaattccagcctgactctgaggagtctggtgcccggctcctcctatgccatgtcagtgtgggcctgggcagagaaccttggctctagcatccagaagatccacccctgtact

SEQ ID NO: 68 - DNA, 1692 bp, Homo sapiens
  modified_base (1)..(1692): “n” represents a, t, c, g, other or unknown
ccgcttgcccctcctctggtaaatgtgacgagtgaaggtcccacccagctctgggcatcctgggtccatgcccccaggggccgagacagctacccggtgaccctgtaccgggcaggcaccagcgccgtcggagccaaggtggccagcacaagcttttcaagtctgactccaggcacgaagtacaaggtggaggttgtcacgcaggctgggccccaccacattgcagcagccaacacctctggctggacccatgaggcatggggggaaggcagcgatgcaggaaaagccctgcacacacccagtgagttggtgtccatgcatgcgagcaccgctgtggtcaacctggcctgggccagcagccccttggggcaggggatgtgctacacccaactctcagaggcggggcacctctcctgggagcaccctctggtgccaggccaagcccacctcatcctgaggggcctcacacctggatgcaacctctccctgtcagtgctgtgccaggcagggccgctgcaggcgtccactcagcgcgtggtactgcttgttgagcctggccctgtggaagatgtgcagtgccagcctgaggccaccttcctggccctgaactggacagtgcccgccagggatgtgggcacctgtctggtggtggcagagcagctggtggcaggagggaatgctcaccttgtgttccaggccgacacctccaaaaatgcagtcctgttgcccaacctggtgcctgtcacttcctatcacctcagcctcgccgtgctgggcaggaacggtctgtggagtcgggtggtcactctggcatgttccacatctgccgaggcctggcatcccccagcgttagccccggcccctgagctggagcctgggacagaaatgggagtgatgatcccgcggggtatgtttggcaaggatgatgggcagatccagtggtacggcatcattgccaccaccaacatgtcactgcctcagccttcctgggaagccatcaaccacatgtggcatgaccactactacagaggacatgactcctacctggccatcctgctccccaaccccttctacccggatccctgggctgtgccgagatcctggacagtgcctgtgggtacagaggactgtggccacaccaaagagatatgcaacgggcagctcaagctaggtcctgtttctctgcccaggttcagcgttgcagcctttaccaggtacagccctcctgagaccattaactccttctcagccttctcgnagccctgggccggtgtctccctggcatcagtgcccctgccggtaatggagggcctcgtggtgggctgtgtcctcaccatctgtgctgtgctgggcctgctgtgctggaggcgggtgaaggggcagagggcagggaagaatccattttcccaagagctgacagcttacaacctgcggtagacccaccggcccatccctatccacagcttcaggcagagctatgaggccaagagtgctcacgcacaccaggctttctttttgcaattcgaggagctgaaggaggtgggcaaggagcagcccagactggaggctgagtacgctgccaacaccaccaagaaccattacccacatgtgcttccctacgaccactccagggtcaggctgacccagctggagggagagcctcattctgactacatcaatgccaacttcatc

SEQ ID NO: 69 - DNA, 333 bp, Homo sapiens
gctacacccacccacccacaggaattcattgcctctcaggcgcctctcaagaaaacgctggagaacttctggcggctggtgcgggagtagcaggtccgcatcatcatcatgctgaccgtcggcatggagaacaggagggtgctgtgtgagcattactggctgaccgactctaccccggtcacccatgatcacatcaccatccacctcctagccgaggaggctgacgatgagtggaccaagcgggaattccagctgcagcacatgcgtgccccaaggatgagggggttgtccagcaacagcagcggagggtggagtaactgcaattcaccacct

SEQ ID NO: 70 - DNA, 1191 bp, Homo sapiens
cctgaccacagcatcctcaaggcccccagctccctgcttacctttatggagctggtacaggaacaggcaagggccacccagggcatgggacccatcctggtgcactgcaggagggcagtgtgggcatggaggcagacgggcaccttcgtggccctgttgaggctgctgcagcagctggaggaggagcagatggtagatgtgttccatgctgtgtttgcattctggatgcacgggcccctcatgatccagaccctgagccagtacgtcttcctgcacagctgcctactgaacaagattctggaagggcccttcaacatctctgagtcttggcccatctctgtgatgaacttcgcacaggcgtgtgccaagagggcagccaatgccaacgctggcttcttgaaggagtacgagctcttgctgcaggccatcaaggacgaggctggctcttacgcacccctgcctggctatgagcaggacagccccatctcctgtgagtctcactgggacaccctcagtctctggaagccaatgagctgtgctctgcagggtgggccctctggctgtgatcatatggtgctgactggcctcgcagggccagaggagctctgggagctggtgtggcagcacggggctcatgtgcttgtctctctgtgcccactcgatgccatggagaagccacaggaattctggccaatggagatgcagcccatagtcacagacatggtgacagtgcactgggtggccgagagcagcacagtgggctggctctgtgccctcttcagggtcacacatgtagcaccaatgccgatcatgtctttgcccgagggggagagtaggaaggaaagggaggtgcagagactgcagtttccatacctggagcctgggcatgagctgcccgccaccaccctgctgcccttcctggctgctgtgggccagtgctgctctcggggcaacagcaagaagccgggcacactgctcagccactccagcaagggtgcgacccagctgggcaccttcctggccatggagcagctgctgcagcaggcagggtctgagtgcaccgtggatgtctttaacgtggccctgcagcagtctcaggcctgtgaccttatgaccccaacgctgaagcagtatatctacctctacaattgtctgaacagcgcactggcagacgggctgcccctgagtcgg

SEQ ID NO: 71 - DNA, 57 bp, Homo sapiens
cactggtcactgtgcaggagaggtttgtgccctgtgggatggggacagcattcctga

SEQ ID NO: 72 - protein, 174 aa, Homo sapiens
DSAAKSRQGSGAKRQLDGLEASKEPGRRALLYTEGNPGLLGNISVPPGATHITFYGPVPGARYCVDIASSLGIITYSLMGHKSPLAPQSLEVISRGGPSDLAIVWAPAPGQREGYRVAWHQEGSQRSPGSLVDLGPDNSSLTLRSLVPGSSYAMSVWAWAENLGSSIQKIHPCT

SEQ ID NO: 73 - protein, 563 aa, Homo sapiens
  MOD_RES (1)..(563): “Xaa” represents any, other or unknown amino acid
PLAPPLVNVTSEGPTQLWASWVHAPRGRDSYPVTLYRAGTSAVGAKVASTSFSSLTPGTKYKVEVVTQAGPHHIAAANTSGWTHEAWGEGSDAGKALHTPSELVSMHASTAVVNLAWASSPLGQGMCYTQLSEAGHLSWEHPLVPGQAHLILRGLTPGCNLSLSVLCQAGPLQASTQRVVLLVEPGPVEDVQCQPEATFLALNWTVPARDVGTCLVVAEQLVAGGNAHLVFQADTSKNAVLLPNLVPVTSYHLSLAVLGRNGLWSRVVTLACSTSAEAWHPPALAPAPELEPGTEMGVMIPRGMFGKDDGQIQWYGIIATTNMSLPQPSWEAINHMWHDHYYRGHDSYLAILLPNPFYPDPWAVPRSWTVPVGTEDCGHTKEICNGQLKLGPVSLPRFSVAAFTRYSPPETINSFSAFSXPWAGVSLASVPLPVMEGLVVGCVLTICAVLGLLCWRRVKGQRAGKNPFSQELTAYNLRTHRPIPIHSFRQSYEAKSAHAHQAFFLQFEELKEVGKEQPRLEAEYAANTTKNHYPHVLPYDHSRVRLTQLEGEPHSDYINANFI

SEQ ID NO: 74 - protein, 110 aa, Homo sapiens [sequence omitted]

SEQ ID NO: 75 - protein, 397 aa, Homo sapiens
PDHSILKAPSSLLTFMELVQEQARATQGMGPILVHCRRAVWAWRQTGTFVALLRLLQQLEEEQMVDVFHAVFAFWMHGPLMIQTLSQYVFLHSCLLNKILEGPFNISESWPISVMNFAQACAKRAANANAGFLKEYELLLQAIKDEAGSYAPLPGYEQDSPISCESHWDTLSLWKPMSCALQGGPSGCDHMVLTGLAGPEELWELVWQHGAHVLVSLCPLDAMEKPQEFWPMEMQPIVTDMVTVHWVAESSTVGWLCALFRVTHVAPMPIMSLPEGESRKEREVQRLQFPYLEPGHELPATTLLPFLAAVGQCCSRGNSKKPGTLLSHSSKGATQLGTFLAMEQLLQQAGSECTVDVFNVALQQSQACDLMTPTLKQYIYLYNCLNSALADGLPLSR

SEQ ID NO: 76 - protein, 18 aa, Homo sapiens
HWSLCRRGLCPVGWGQHS

(19) United States
(12) Patent Application Publication
(10) Pub. No.: US 2005/0084877 A1
(43) Pub. Date: Apr. 21, 2005

(54) Mammalian protein phosphatases

(75) Inventors: Gregory D. Plowman, San carlos, CA (US); Ricardo Martinez, Foster City, CA (US); David Whyte, Belmont, CA (US); Gerard Manning, Menlo Park, CA (US); Sucha Sudarsanam, Greenbrae, CA (US); Ronald Hill, Burlingame, CA (US); Peter Flanagan, San Francisco, CA (US)

Correspondence Address: FOLEY AND LARDNER;SUITE 500, 3000 K STREET NW, WASHINGTON, DC, 20007, US

(73) Assignee: SUGEN INC.

(21) Appl. No.: 10/838,181

(22) Filed: May 4, 2004

Related U.S. Application Data
(62) Division of application No. 10/168,506, filed on Oct. 10, 2002.
(60) Provisional application No. 60/173,255, filed on Dec. 21, 1999.
(60) Provisional application No. 60/175,766, filed on Dec. 28, 1999.
(60) Provisional application No. 60/178,078, filed on Jan. 25, 2000.
(60) Provisional application No. 60/179,301, filed on Jan. 31, 2000.

(51) Int. Cl. C12Q 1/68; C07H 21/04; C12N 9/16

(52) U.S. Cl. 435/6; 435/69.1; 435/196; 435/320.1; 435/325; 536/23.2

(57) ABSTRACT

The present invention relates to phosphatase polypeptides having the amino acid sequence of SEQ ID NO:23 or SEQ ID NO:24, disclosed herein. The invention also relates to nucleotide sequences encoding the phosphatase polypeptides, vectors and recombinant cells comprising such sequences, antibodies having binding affinity to the phosphatase polypeptides, and other products. Included in the invention are methods for identifying modulators of the phosphatase polypeptides, as well as methods for diagnosing and treating various conditions related to the phosphatase polypeptides.

The present invention claims priority on provisional application Ser. Nos. 60/173,255, 60/178,078, 60/179,301, 60/175,766, (and the provisional application Ser. No. represented by Sugen docket no. “Cel_{—}16”, all of which are hereby incorporated by reference in their entirety.

### FIELD OF THE INVENTION

The present invention relates to phosphatase polypeptides, nucleotide sequences encoding the phosphatase polypeptides, as well as various products and methods useful for the diagnosis and treatment of various phosphatase-related diseases and conditions.

### BACKGROUND OF THE INVENTION

The following description of the background of the invention is provided to aid in understanding the invention, but is not admitted to be or to describe prior art to the invention.

Cellular sign transduction is a fundamental mechanism whereby external stimuli that regulate diverse cellular processes are relayed to the interior of cells. One of the key biochemical mechanisms of signal transduction involves the reversible phosphorylation of proteins by protein kinases, which enables regulation of the activity of mature proteins by altering their structure and function. The best characterized protein kinases in eukaryotes phosphorylate proteins on the alcohol moiety of serine, threonine and tyrosine residues. These kinases largely fall into two groups: those specific for phosphorylating seines and threonines, and those specific for phosphorylating tyrosines.

The phosphorylation state of a given substrate is also regulated by the protein phosphatases, a class of proteins responsible for removal of the phosphate group added to a given substrate by a protein kinase. The protein phosphatases can also be classified as being specific for either serine/threonine or tyrosine. Some members of this family are able to dephosphorylate only tyrosine, and are known as the “protein tyrosine phosphatases” (“PTP”); while others are able to dephosphorylate tyrosine as well as serine and threonine, and are named, “dual-specificity phosphatases” (“DSP”); and a third family dephosphorylates only serine or threonine (“STP”)—as disclosed by Fauman et al., Trends Biochem. Sci. November 1996;21(11):413-7; and Martell et al., Mol. Cells. 1998 Feb. 28;8(1): 2-11. These proteins share a 250-300 amino acid domain that comprises the common catalytic core structure. Related phosphatases are clustered into distinct subfamilies of tyrosine phosphatases, dual-specificity phosphatases, and myotubularin-like phosphatases (Fauman et al., supra; and Martell et al, supra).

Phosphatases possess a variety of non-catalytic domains that are believed to interact with upstream regulators. Examples include proline-rich domains for interaction with SH3-containing proteins, or specific domains for interaction with Rac, Rho, and Rab small G-proteins. These interactions may provide a mechanism for cross-talk between distinct biochemical pathways in response to external stimuli such as the activation of a variety of cell surface receptors, including tyrosine kinases, cytokine receptors, TNF receptor, Fas, T cell receptors, CD28, or CD40.

Phosphatases have been implicated as regulating a variety of cellular responses, including response to growth factors, cytokines and hormones, oxidative-, UV-, or irradiation-related stress pathways, inflammatory signals (e.g. TNFα), apoptotic stimuli (e.g. Fas), T and B cell costimulation, the control of cytoskeletal architecture, and cellular transformation (see THE PROTEIN PHOSPHATASE FACTBOOK, Tonks et al., Academic Press, 2000).

A need, therefore, exists to identify additional phosphatases whose inappropriate activity may lead to cancer or other disorders so that appropriate treatments for those disorders might also be identified.

### SUMMARY OF THE INVENTION

The following abbreviations are use to describe characeristics of the phosphatases according to the invention:
DsPTP Dual specificity protein phosphatase
DUS Dual specificity phosphatase
MKP MAP Kinase phosphatase
MTM Myotubular myopathy (myotubularin) phosphatase
PTP Protein Tyrosine Phosphatase
PTEN Phosphatase and tensin homolog

Through the use of a “motif extraction” bioinformatics script, the named inventors have identified certain mammalian members of the phosphatase family, which are disclosed herein. The invention provides a partial or complete sequence of 12 phosphatases, as well as the classification, predicted or deduced protein structure, and a strategy for elucidating the biologic and therapeutic relevance of these proteins. These novel proteins include: eight (8) MAP kinase phosphatase enzymes (“MKPs”), which are members of the DSP family; two (2) phosphatases from the STP family, and two (2) phosphatases from the PTP family. The classification of novel proteins as belonging to established families has proven highly accurate, not only in predicting motifs present in the remaining non-catalytic portion of each protein, but also in the regulation, substrates and signaling pathways for these proteins.

One aspect of the invention features an identified, isolated, enriched, or purified nucleic acid molecule encoding a phosphatase polypeptide, having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

By “isolated” in reference to nucleic acid is meant a polymer of 10 (preferably 21, more preferably 39, most preferably 75) or more nucleotides conjugated to each other, including DNA and RNA that is isolated from a natural source or that is synthesized as the sense or complementary antisense strand. In certain embodiments of the invention, longer nucleic acids are preferred, for example those of 300, 600, 900, 1200, 1500, or more nucleotides and/or those having at least 50%, 60%, 75%, 90%, 95% or 99% identity to a sequence selected from the group consisting of those set forthin SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

The isolated nucleic acid of the present invention is unique in the sense that it is not found in a pure or separated state in nature. Use ofthe term “isolated” indicates that a naturally occurring sequence has been removed from its normal cellular (i.e., chromosomal) environment Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2- to 5-fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form. The term “purified” in reference to nucleic acid does not require absolute purity (such as a homogeneous preparation). Instead, it represents an indication that the sequence is relatively more pure than in the natural environment (compared to the natural level this level should be at least 2- to 5-fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “phosphatase polypeptide” is meant 32 (preferably 40, more preferably 45, most preferably 55) or more contiguous amino acids in a polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. In certain aspects, polypeptides of 100, 200, 300, 400, 450, 500, 550, 600, 700, 800, 900 or more amino acids are preferred. The phosphatase polypeptide can be encoded by a full-length nucleic acid sequence or any portion of the full-length nucleic acid sequence, so long as a functional activity of the polypeptide is retained. It is well known in the art that due to the degeneracy of the genetic code numerous different nucleic acid sequences can code for the same amino acid sequence. Equally, it is also well known in the art that conservative changes in amino acid can be made to arrive at a protein or polypeptide which retains the functionality of the original. Such substitutions may include the replacement of an amino acid by a residue having similar physicochemical properties, such as substituting one aliphatic residue (Ile, Val, Leu or Ala) for another, or substitution between basic residues Lys and Arg, acidic residues Glu and Asp, amide residues Gln and Asn, hydroxyl residues Ser and Tyr, or aromatic residues Phe and Tyr. Further information regarding making amino acid exchanges which have only slight, if any, effects on the overall protein can be found in Bowie et al., Science, 1990, 247:1306-1310, which is incorporated herein by reference in its entirety including any figures, tables, or drawings. In all cases, all permutations are intended to be covered by this disclosure.

The amino acid sequence of the phosphatase peptide of the invention will be substantially similar to a sequence having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, or the corresponding full-length amino acid sequence or fragments thereof.

A sequence that is substantially similar to a sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24 will preferably have at least 90% identity (more preferably at least 95% and most preferably 99-100%) to the sequence.

By “identity” is meant a property of sequences that measures their similarity or relationship. Identity is measured by dividing the number of identical residues by the total number of residues and gaps and multiplying the product by 100. “Gaps” are spaces in an alignment that are the result of additions or deletions of amino acids. Thus, two copies of exactly the same sequence have 100% identity, but sequences that are less highly conserved, and have deletions, additions, or replacements, may have a lower degree of identity. Those skilled in the art will recognize that several computer programs are available for determining sequence identity using standard parameters for example Gapped BLAST or PSI-BLAST (Altschul, et al. (1997) Nucleic Acids Res. 25:3389-3402), BLAST (Altschul et al. (1990) J. Mol. Biol. 215:403-410), and Smith-Waterman (Smith, et al. (1981) J. Mol. Biol. 147:195-197). Preferably, the default settings of these programs will be employed, but those skilled in the art recognize whether these settings need to be changed and know how to make the changes.

“Similarity” is measured by dividing the number of identical residues plus the number of conservatively substituted residues (see Bowie, et al. Science, 1999 247:1306-1310, which is incorporated here by reference in its entirety, including any drawing figures, or tables) by the total number of residues and gaps and multiplying the product by 100.

In preferred embodiments, the invention features isolated, enriched, or purified nucleic acid molecules encoding a phosphatase polypeptide comprising a nucleotide sequence that (a) encodes a polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24; (b) is the complement of the nucleotide sequence of (a); (c) hybridizes under highly stringent conditions to the nucleotide molecule of (a) and encodes a naturally occurring phosphatase polypeptide; (d) encodes a polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, except that it lacks one or more, but not all, of the domains selected from the group consisting of an N-terminal domain, a catalytic domain, a C-terminal catalytic domain, a C-terminal domain, a coiled-coil structure region, a proline-rich region, a spacer region, and a C-terminal tail; and (e) is the complement of the nucleotide sequence of (d).

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence.

Various low or high stringency hybridization conditions may be used depending upon the specificity and selectivity desired. These conditions are well known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the full-length sequence.

By stringent hybridization assay conditions is meant hybridization assay conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhardt's solution at 42° C. overnight; washing with 2×SSC, 0.1% SDS at 45° C.; and washing with 0.2×SSC, 0.1% SDS at 45° C. Under some of the most stringent hybridization assay conditions, the second wash can be done with 0.1×SSC at a temperature up to 70° C. (Berger et al. (1987) Guide to Molecular Cloning Techniques pg 421, hereby incorporated by reference herein in its entirety including any figures, tables, or drawings.). However, other applications may require the use of conditions falling between these sets of conditions. Methods of determining the conditions required to achieve desired hybridizations are well known to those with ordinary skill in the art and are based on several factors, including but not limited to, the sequences to be hybridized and the samples to be tested. Washing conditions of lower stringency frequently utilize a lower temperature during the washing steps, such as 65° C., 60° C., 55° C., 50° C., or 42° C.

The term “domain” refers to a region of a polypeptide which serves a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The term “N-terminal domain” refers to the extracatalytic region located between the initiator methionine and the catalytic domain of the protein phosphatase. The N-terminal domain can be identified following a Smith-Waterman alignment of the protein sequence against the non-redundant protein database to define the N-terminal boundary of the catalytic domain. Depending on its length, the N-terminal domain may or may not play a regulatory role in phosphatase function. The term “catalytic domain” refers to a region of the protein phosphatase that is typically 25-300 amino acids long and is responsible for carrying out the phosphate transfer reaction from a high-energy phosphate donor molecule such as ATP or GTP to itself (autophosphorylation) or to other proteins (exogenous phosphorylation). The catalytic domain of protein phosphatases is made up of 12 subdomains a contain highly conserved amino acid residues, and are responsible for proper polypeptide folding and for catalysis. The catalytic domain can be identified following a Smith-Waterman alignment of the protein sequence against the non-redundant protein database.

The term “catalytic activity”, as used herein, defines the rate at which a phosphatase catalytic domain phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active site of a protein phosphatase. The active site is normally a cavity in which the substrate binds to the protein phosphatase and is phosphorylated.

The term “substrate” as used herein refers to a molecule phosphorylated by a phosphatase of the invention. Phosphatases phosphorylate substrates on serine/threonine or tyrosine amino acids. The molecule may be another protein or a polypeptide.

The term “C-terminal domain” refers to the region located between the catalytic domain or the last (located closest to the C-terminus) functional domain and the carboxy-terminal amino acid residue of the protein phosphatase. By “functional” domain is meant any region of the polypeptide that may play a regulatory or catalytic role as predicted from amino acid sequence homology to other proteins or by the presence of amino acid sequences that may give rise to specific structural conformations (e.g. N-terminal domain). The C-terminal domain can be identified by using a Smith-Waterman alignment of the protein sequence against the non-redundant protein database to define the C-terminal boundary of the catalytic domain or of any functional C-terminal extracatalytic domain. Depending on its length and amino acid composition, the C-terminal domain may or may not play a regulatory role in phosphatase function. For the some of the phosphatases of the instant invention, the C-terminal domain may also comprise the catalytic domain (above).

The term “C-terminal tail” as used herein, refers to a C-terminal domain of a protein phosphatase, that by homology extends or protrudes past the C-terminal amino acid of its closest homolog. C-terminal tails can be identified by using a Smith-Waterman sequence alignment of the protein sequence against the non-redundant protein database, or by means of a multiple sequence alignment of homologous sequences using the DNAStar program Megalign. Depending on its length, a C-terminal tail may or may not play a regulatory role in phosphatase function.

The term “coiled-coil structure region” as used herein, refers to a polypeptide sequence that has a high probability of adopting a coiled-coil structure as predicted by computer algorithms such as COILS (Lupas, A. (1996) Meth. Enzymology 266:513-525). Coiled-coils are formed by two or three amphipathic α-helices in parallel. Coiled-coils can bind to coiled-coil domains of other polypeptides resulting in homo- or heterodimers (Lupas, A. (1991) Science 252:1162-1164).

The term “proline-rich region” as used herein, refers to a region of a protein phosphatase whose proline content over a given amino acid length is higher than the average content of this amino acid found in proteins (i.e., >10%). Proline-rich regions are easily discernable by visual inspection of amino acid sequences and quantitated by standard computer sequence analysis programs such as the DNAStar program EditSeq. Proline-rich regions have been demonstrated to participate in regulatory protein-protein interactions.

The term “spacer region” as used herein, refers to a region of the protein phosphatase located between predicted functional domains. The spacer region has no detectable homology to any amino acid sequence in the database, and can be identified by using a Smith-Waterman alignment of the protein sequence against the non-redundant protein database to define the C- and N-terminal boundaries of the flanking functional domains. Spacer regions may or may not play a fundamental role in protein phosphate function.

The term “insert” as used herein refers to a portion of a protein phosphatase that is absent from a close homolog. Inserts may or may not by the product alternative splicing of exons. Inserts can be identified by using a Smith-Waterman sequence alignment of the protein sequence against the non-redundant protein database, or by means of a multiple sequence alignment of homologous sequences using the DNAStar program Megalign. Inserts may play a functional role by presenting a new interface for protein-protein interactions, or by interfering with such interactions.

The term “signal transduction pathway” refers to the molecules that propagate an extracellular signal through the cell membrane to become an intracellular signal. This signal can then stimulate a cellular response. The polypeptide molecules involved in signal seduction processes are typically receptor and non-receptor protein tyrosine phosphatases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors.

In other preferred embodiments, the invention features isolated, enriched, or purified nucleic acid molecules encoding phosphatase polypeptides, further comprising a vector or promoter effective to initiate transcription in a host cell. The invention also features recombinant nucleic acid, preferably in a cell or an organism. The recombinant nucleic acid may contain a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO: 12, or a functional derivative thereof, and a vector or a promoter effective to initiate transcription in a host cell. The recombinant nucleic acid can alternatively contain a transcriptional initiation region functional in a cell, a sequence complementary to an RNA sequence encoding a phosphatase polypeptide and a transcriptional termination region functional in a cell. Specific vectors and host cell combinations are discussed herein.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a phosphatase can be inserted into a vector by cutting the vector with restriction enzymes and ligating the two pieces together.

The term “transfecting” defines a number of methods to insert a nucleic acid vector or other nucleic acid molecules into a cellular organism. These methods involve a variety of techniques, such as treating the cells with high concentrations of salt, an electric field, detergent or DMSO to render the outer membranes or wall of the cells permeable to nucleic acid molecules of interest or use of various viral transduction strategies.

The term “promoters” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, which encodes an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, a functional derivative thereof or at least 35, 40, 45, 50, 60, 75, 100, 200, or 300 contiguous amino acids selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. The nucleic acid may be isolated from a oral source by cDNA cloning or by subtractive hybridization. The natural source may be mammalian, preferably human, blood, semen, or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a phosphatase polypeptide, to which a particular nucleic acid sequence can hybridize under lower stringency conditions. Examples of lower stringency conditions suitable for screening for nucleic acid encoding phosphatase polypeptides are provided in Wahl et al. Meth. Enzym. 152:399-407 (1987) and in Wahl et al. Meth Enzym. 152:415-423 (1987), which are hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a phosphatase polypeptide that is not present in a sequence coding for any other naturally occurring polypeptide. Such regions preferably encode 32 (preferably 40, more preferably 45, most preferably 55) or more contiguous amino acids set forth in a full-length amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. In particular, a unique nucleic acid region is preferably of mammalian origin.

Another aspect of the invention features a nucleic acid probe for the detection of nucleic acid encoding a phosphatase polypeptide having a amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24 in a sample. The nucleic acid probe contains a nucleotide base sequence that will hybridize to the sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO: 12, or a functional derivative thereof.

In preferred embodiments, the nucleic acid probe hybridizes to nucleic acid encoding at least 12, 32, 75, 90, 105, 120, 150, 200, 250, 300 or 350 contiguous amino acids of a full-length sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, or a functional derivative thereof.

Methods for using the probes include detecting the presence or amount of phosphatase RNA in a sample by contacting the sample with a nucleic acid probe under conditions such that hybridization occurs and detecting the presence or amount of the probe bound to phosphatase RNA. The nucleic acid duplex formed between the probe and a nucleic acid sequence coding for a phosphatase polypeptide may be used in the identification of the sequence of the nucleic acid detected (Nelson et al., in Nonisotopic DNA Probe Techniques, Academic Press, San Diego, Kricka, ed., p. 275, 1992, hereby incorporated by reference herein in its entirety, including any drawings, figures, or tables). Kits for performing such methods may be constructed to include a container means having disposed therein a nucleic acid probe.

In another aspect, the invention describes a recombinant cell or tissue comprising a nucleic acid molecule encoding a phosphatase polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the phosphatase polypeptides.

The polypeptide is preferably a fragment of the protein encoded by a full-length amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. By “fragment is meant an amino acid sequence present in a phosphatase polypeptide. Preferably, such a sequence comprises at least 32, 45, 50, 60, 100, 200, or 300 contiguous amino acids of a full-length sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

In another aspect, the invention features an isolated, enriched, or purified phosphatase polypeptide having the amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

By “isolated” in reference to a polypeptide is meant a polymer of 6 (preferably 12, more preferably 18, most preferably 25, 32, 40, or 50) or more amino acids conjugated to each other, including polypeptides that are isolated from a natural source or that are synthesized. In certain aspects, longer polypeptides are preferred, such as those with 100, 200, 300, 400, 450, 500, 550, 600, 700, 800, 900 or more contiguous amino acids of a full-length sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid-based material naturally associated with it.

By the use of the term “enriched” in reference to a polypeptide is meant that the specific amino acid sequence constitutes a significantly higher fraction (2- to 5-fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term significant here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-to 5-fold greater (e.g., in terms of mg/mL). Purification of at least one order of magnitude, preferably two or three orders, and more preferably four or five orders of magnitude is expressly contemplated. The substance is preferably free of contamination at a functionally significant level for example 90%, 95%, or 99% pure.

In preferred embodiments, the phosphatase polypeptide is a fragment of the protein encoded by a full-length amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. Preferably, the phosphatase polypeptide contains at least 32,45,50,60,100,200, or 300 contiguous amino acids of a full-length sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, or a functional derivative thereof.

In preferred embodiments, the phosphatase polypeptide comprises an amino acid sequence having (a) an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24; and (b) an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, except that it lacks one or more of the domains selected from the group consisting of a C-terminal catalytic domain an N-terminal domain, a catalytic domain, a C-terminal domain, a coiled-coil structure region, a proline-rich region, a spacer region, and a C-terminal tail.

The polypeptide can be isolated from a natural source by methods well-known in the art. The natural source may be mammalian, preferably human, blood, semen, or tissue, and the polypeptide may be synthesized using an automated polypeptide synthesizer.

In some embodiments the invention includes a recombinant phosphatase polypeptide having (a) an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. By “recombinant phosphatase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

The polypeptides to be expressed in host cells may also be fusion proteins which include regions from heterologous proteins. Such regions may be included to allow, e.g., secretion, improved stability, or facilitated purification of the polypeptide. For example, a sequence encoding an appropriate signal peptide can be incorporated into expression vectors. A DNA sequence for a signal peptide (secretory leader) may be fused in-frame to the polynucleotide sequence so that the polypeptide is translated as a fusion protein comprising the signal peptide. A signal peptide that is functional in the intended host cell promotes extracellular secretion of the polypeptide. Preferably, the signal sequence will be cleaved from the polypeptide upon secretion of the polypeptide from the cell. Thus, preferred fusion proteins can be produced in which the N-terminus of a phosphatase polypeptide is fused to a carrier peptide.

In one embodiment, the polypeptide comprises a fusion protein which includes a heterologous region used to facilitate purification of the polypeptide. Many of the available peptides used for such a function allow selective binding of the fusion protein to a binding partner. A preferred binding partner includes one or more of the IgG binding domains of protein A are easily purified to homogeneity by affinity chromatography on, for example, IgG-coupled Sepharose. Alternatively, many vectors have the advantage of carrying a stretch of histidine residues that can be expressed at the N-terminal or C-terminal end of the target protein, and thus the protein of interest can be recovered by metal chelation chromatography. A nucleotide sequence encoding a recognition site for a proteolytic enzyme such as enterophosphatase, factor X procollagenase or thrombin may immediately precede the sequence for a phosphatase polypeptide to permit cleavage of the fusion protein to obtain the mature phosphatase polypeptide. Additional examples of fusion-protein binding partners include, but are not limited to, the yeast I-factor, the honeybee melatin leader in sf9 insect cells, 6-His tag, thioredoxin tag, hemaglutinin tag, GST tag, and OmpA signal sequence tag. As will be under by one of skill in the art, the binding partner which recognizes and binds to the peptide may be any ion, molecule or compound including metal ions (e.g., metal affinity columns), antibodies, or fragments thereof, and any protein or peptide which binds the peptide, such as the FLAG tag.

In another aspect, the invention features an antibody (e.g., a monoclonal or polyclonal antibody) having specific binding affinity to a phosphatase polypeptide or a phosphatase polypeptide domain or fragment where the polypeptide is selected from the group having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. By “specific binding affinity” is meant that the antibody binds to the target phosphatase polypeptide with greater affinity than it binds to other polypeptides under specified conditions. Antibodies or antibody fragments are polypeptides that contain regions that can bind other polypeptides. The term “specific binding affinity” describes an antibody that binds to a phosphatase polypeptide with greater affinity than it binds to other polypeptides under specified conditions. Antibodies can be used to identify an endogenous source of phosphatase polypeptides, to monitor cell cycle regulation, and for immuno-localization of phosphatase polypeptides within the cell.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et a., Nature 256:495-497, 1975, and U.S. Pat. No. 4,376,110, both of which are hereby incorporated by reference herein in their entirety including any figures, tables, or drawings).

The term “antibody fragment” refers to a portion of an antibody, often the hypervariable region and portions of the surrounding heavy and light chains, that displays specific binding affinity for a particular molecule. A hypervariable region is a portion of an antibody that physically binds to the polypeptide target.

Antibodies or antibody framents having specific binding affinity to a phosphatase polypeptide of the invention may be used in methods for detecting the presence and/or amount of phosphatase polypeptide in a sample by probing the sample with the antibody under conditions suitable for phosphatase-antibody immunocomplex formation and detecting the presence and/or amount of the antibody conjugated to the phosphatase polypeptide. Diagnostic kits for performing such methods may be constructed to include antibodies or antibody fragments specific for the phosphatase as well as a conjugate of a binding partner of the antibodies or the antibodies themselves.

An antibody or antibody fragment with specific binding affinity to a phosphatase polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a phosphatase polypeptide of the invention may be used in methods for detecting the presence and/or amount of phosphatase polypeptide in a sample by contacting the sample with the antibody under conditions such that an immunocomplex forms and detecting the presence and/or amount of the antibody conjugated to the phosphatase polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In another aspect, the invention features a hybridoma which produces an antibody having specific binding affinity to a phosphatase polypeptide or a phosphatase polypeptide domain, where the polypeptide is selected from the group having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. By “hybridoma” is meant an immortalized cell line that is capable of secreting an antibody, for example an antibody to a phosphatase of the invention. In preferred embodiments, the antibody to the phosphatase comprises a sequence of amino acids that is able to specifically bind a phosphatase polypeptide of the invention.

In another aspect, the present invention is also directed to kits comprising antibodies that bind to a polypeptide encoded by any of the nucleic acid molecules described above, and a negative control antibody.

The term “negative control antibody” refers to an antibody derived from similar source as the antibody having specific binding affinity, but where it displays no binding affinity to a polypeptide of the invention.

In another aspect, the invention features a phosphatase polypeptide binding agent able to bind to a phosphatase polypeptide selected from the group having (a) an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. The binding agent is preferably a purified antibody that recognizes an epitope preset on a phosphatase polypeptide of the invention. Other binding agents include molecules that bind to phosphatase polypeptides and analogous molecules that bind to a phosphatase polypeptide. Such binding agents may be identified by using assays that measure phosphatase binding partner activity.

The invention also features a method for screening for human cells containing a phosphatase polypeptide of the invention or an equivalent sequence. The method involves identifying the novel polypeptide in human cells using techniques that are routine and standard in the art, such as those described herein for identifying the phosphatases of the invention (e.g., cloning, Southern or Northern blot analysis, in situ hybridization, PCR amplification, etc.).

In another aspect, the invention features methods for identifying a substance that modulates phosphatase activity comprising the steps of: (a) contacting a phosphatase polypeptide selected from the group having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24 with a test substance; (b) measuring the activity of said polypeptide; and (c) determining whether said substance modulates the activity of said polypeptide.

The term “modulates” refers to the ability of a compound to alter the function of a phosphatase of the invention. A modulator preferably activates or inhibits the activity of a phosphatase of the invention depending on the concentration of the compound exposed to the phosphatase.

The term “modulates” also refers to altering the function of phosphatases of the invention by increasing or decreasing the probability that a complex forms between the phosphatase and a natural binding partner. A modulator preferably increases the probability that such a complex forms between the phosphatase and the natural binding partner, more preferably increases or decreases the probability that a complex forms between the phosphatase and the natural binding partner depending on the concentration of the compound exposed to the phosphatase, and most preferably decreases the probability that a complex forms between the phosphatase and the natural binding partner.

The term “activates” refers to increasing the cellular activity of the phosphatase. The term inhibit refers to decreasing the cellular activity of the phosphatase. Phosphatase activity is preferably the interaction with a natural binding partner.

The term “complex” refers to an assembly of at least two molecules bound to one another. Signal transduction complexes often contain at least two protein molecules bound to one another.

The term “natural binding partner” refers to polypeptides, lipids, small molecules, or nucleic acids that bind to phosphatases in cells. A change in the interaction between a phosphatase and a natural binding partner can manifest itself as an increased or decreased probability that the interaction forms, or an increased or decreased concentration of phosphatase/natural binding partner complex.

The term “contacting” as used herein refers to mixing a solution comprising the test compound with a liquid medium bathing the cells of the methods. The solution comprising the compound may also comprise another component, such as dimethyl sulfoxide (DMSO), which facilitates the uptake of the test compound or compounds into the cells of the methods. The solution comprising the test compound may be added to the medium bathing the cells by utilizing a delivery apparatus, such as a pipette-based device or syringe-based device.

In another aspect, the invention features methods for identifying a substance that modulates phosphatase activity in a cell comprising the steps of: (a) expressing a phosphatase polypeptide in a cell, wherein said polypeptide is selected from the group having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24; (b) adding a test substance to said cell; and (c) monitoring a change in cell phenotype or the interaction between said polypeptide and a natural binding partner.

The term “expressing” as used herein refers to the production of phosphatases of the invention from a nucleic acid vector containing phosphatase genes within a cell. The nucleic acid vector is transfected into cells using well known techniques in the art as described herein.

Another aspect of the instant invention is directed to methods of identifying compounds that bind to phosphatase polypeptides of the present invention, comprising contacting the phosphatase polypeptides with a compound, and determining whether the compound binds the phosphatase polypeptides. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology, 1999, John Wiley & Sons, NY, which is incorporated herein by reference in its entirety. The compounds to be screened include, but are not limited to, compounds of extracellular, intracellular, biological or chemical origin.

The methods of the invention also embrace compounds that are attached to a label, such as a radiolabel (e.g., ¹²⁵I, ³⁵S, ³²P, ³³P, ³H), a fluorescence label a chemiluminescent label an enzymic label and an immunogenic label. The phosphatase polypeptides employed in such a test may either be free in solution, attached to a solid support, borne on a cell surface, located intracellularly or associated with a portion of a cell. One skilled in the art can, for example, measure the formation of complexes between a phosphatase polypeptide and the compound being tested. Alternatively, one skilled in the art can examine the diminution in complex formation between a phosphatase polypeptide and its substrate caused by the compound being tested.

Other assay can be used to examine enzymatic activity including, but not limited to, photometric, radiometric, HPLC, electrochemical, and the like, which are described in, for example, Enzyme Assays: A Practical Approach, eds. R. Eisenthal and M. J. Danson, 1992, Oxford University Press, which is incorporated herein by reference in its entirety.

Another aspect of the present invention is directed to methods of identifying compounds which modulate (i.e., increase or decrease) activity of a phosphatase polypeptide comprising contacting the phosphatase polypeptide with a compound, and determining whether the compound modifies activity of the phosphatase polypeptide. These compounds are also referred to as “modulators of protein phosphatases.” The activity in the presence of the test compound is measured to the activity in the absence of the test compound. Where the activity of a sample containing the test compound is higher than the activity in a sample lacking the test compound, the compound will have increased the activity. Similarly, where the activity of a sample containing the test compound is lower than the activity in the sample lacking the test compound, the compound will have inhibited the activity.

The present invention is particularly useful for screening compounds by using a phosphatase polypeptide in any of a variety of drug screening techniques. The compounds to be screened include, but are not limited to, extracellular, intracellular, biological or chemical origin. The phosphatase polypeptide employed in such a test may be in any form, preferably, free in solution, attached to a solid support, borne on a cell surface or located intracellularly. One skilled in the art can, for example, measure the formation of complexes between a phosphatase polypeptide and the compound being tested. Alternatively, one skilled in the art can examine the diminution in complex formation between a phosphatase polypeptide and its substrate caused by the compound being tested.

The activity of phosphatase polypeptides of the invention can be determined by, for example, examining the ability to bind or be activated by chemically synthesised peptide ligands. Alternatively, the activity of the phosphatase polypeptides can be assayed by examining their ability to bind metal ions such as calcium, hormones, chemokines, neuropeptides, neurotransmitters, nucleotides, lipids, odorants, and photons. Thus, modulators of the phosphatase polypeptide's activity may alter a phosphatase function, such as a binding property of a phosphatase or an activity such as signal transduction or membrane localization.

In various embodiments of the method, the assay may take the form of a yeast growth assay, an Aequorin assay, a Luciferase assay, a mitogenesis assay, a MAP Phosphatase activity assay, as well as other binding or function-based assays of phosphatase activity that are generally known in the art. In several of these embodiments, the invention includes any of the receptor and non-receptor protein tyrosine phosphatases, receptor and non-receptor protein phosphatases, polypeptides containing SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), GTPases, phosphodiesterases, phospholipases, prolyl isomerases, proteases, Ca2+ binding proteins, cAMP binding proteins, guanyl cyclases, adenylyl cyclases, NO generating proteins, nucleotide exchange factors, and transcription factors. Biological activities of phosphatases according to the invention include, but are not limited to, the binding of a natural or a synthetic ligand, as well as any one of the functional activities of phosphatases known in the art. Non-limiting examples of phosphatase activities include transmembrane signaling of various forms, which may involve phosphatase binding interactions and/or the exertion of an influence over signal transduction.

The modulators of the invention exhibit a variety of chemical structures, which can be generally grouped into mimetics of natural phosphatase ligands, and peptide and non-peptide allosteric effectors of phosphatases. The invention does not restrict the sources for suitable modulators, which may be obtained from natural sources such as plant, animal or mineral extracts or non-natural sources such as small molecule libraries, including the products of combinatorial chemical approaches to library construction, and peptide libraries.

The use of cDNAs encoding phosphatases in drug discovery programs is well-known; assays capable of testing thousands of unknown compounds per day in high-throughput screens (HTSs) are thoroughly documented. The literature is replete with examples of the use of radiolabelled ligands in HTS binding assays for drug discovery (see Williams, Medicinal Research Reviews, 1991, 11, 147-184.; Sweetnam, et al., J. Natural Products, 1993, 56, 441-455 for review). Recombinant receptors are preferred for binding assay HTS because they allow for better specificity (higher relative purity), provide the ability to generate large amounts of receptor material, and can be used in a broad variety of formats (see Hodgson, Bio/Technology, 1992, 10, 973-980; each of which is incorporated herein by reference in its entirety).

A variety of heterologous systems is available for functional expression of recombinant receptors that are well known to those skilled in the art. Such systems include bacteria (Strosberg et al., Trends in Pharmacological Sciences, 1992, 13, 95-98), yeast (Pausch, Trends in Biotechnology, 1997, 15, 487-494), several kinds of insect cells (Vanden Broeck, Int. Rev. Cytology, 1996, 164, 189-268), amphibian cells (Jayawickreme et al., Current Opinion in Biotechnology, 1997, 8, 629-634) and several mammalian cell lines (CHO, HEK293, COS, etc.; see Gerhardt, et al., Eur. J. Pharmacology, 1997, 334, 1-23). These examples do not preclude the use of other possible cell expression systems, including cell lines obtained from nematodes (PCT application WO 98/37177).

An expressed phosphatase can be used for HTS binding assays in conjunction with its defined ligand, in this case the corresponding peptide that activates it. The identified peptide is labeled with a suitable radioisotope, including, but not limited to, ¹²⁵I, ³H, ³⁵S, or ³²P, by methods that are well known to those skilled in the art. Alternatively, the peptides may be labeled by well-known methods with a suitable fluorescent derivative (Baindur, et al., Drug Dev. Res., 1994, 33, 373-398; Rogers, Drug Discovery Today, 1997, 2, 156-160). Radioactive ligand specifically bound to the receptor in membrane preparations made from the cell line expressing the recombinant protein can be detected in HTS assays in one of several standard ways, including filtration of the receptor-ligand complex to separate bound ligand from unbound ligand (Williams, Med Res. Rev., 1991, 11, 147-184.; Sweetnam, et al., J. Natural Products, 1993, 56, 441-455). Alternative methods include a scintillation proximity assay (SPA) or a FlashPlate format in which such separation is unnecessary (Nakayama, Cur. Opinion Drug Disc. Dev., 1998, 1, 85-91 Bossé, et al., J. Biomolecular Screening, 1998, 3, 285-292.). Binding of fluorescent ligands can be detected in various ways, including fluorescence energy transfer (FRET), direct spectrophotofluorometric analysis of bound ligand, or fluorescence polarization (Rogers, Drug Discovery Today, 1997, 2, 156-160; Hill, Cur. Opinion Drug Disc. Dev., 1998, 1, 92-97).

The phosphatases and natural binding partners required for functional expression of heterologous phosphatase polypeptides can be native constituents of the host cell or can be introduced through well-known recombinant technology. The phosphatase polypeptides can be intact or chimeric. The phosphatase activation results in the stimulation or inhibition of other native proteins, events that can be linked to a measurable response.

Examples of such biological responses include, but are not limited to, the following: the ability to survive in the absence of a limiting nutrient in specifically engineered yeast cells (Pausch, Trends in Biotechnology, 1997, 15, 487-494); changes in intracellular Ca²⁺ concentration as measured by fluorescent dyes Murphy, et al., Cur. Opinion Drug Disc. Dev., 1998, 1, 192-199). Fluorescence changes can also be used to monitor ligand-induced changes in membrane potential or intracellular pH; an automated system suitable for HTS has been described for these purposes (Schroeder, et al., J. Biomolecular Screening, 1996, 1, 75-80).

Assays are also available for the measurement of common second but these are not generally preferred for HTS.

The invention contemplates a multitude of assays to screen and identify inhibitors of ligand binding to phosphatase polypeptides. In one example, the phosphatase polypeptide is immobilized and interaction with a binding partner is assessed in the presence and absence of a candidate modulator such as an inhibitor compound. In another example, interaction between the phosphatase polypeptide and its binding partner is assessed in a solution assay, both in the presence and absence of a candidate inhibitor compound. In either assay, an inhibitor is identified as a compound that decreases binding between the phosphatase polypeptide and its natural binding partner. Another contemplated assay involves a variation of the di-hybrid assay wherein an inhibitor of protein/protein interactions is identified by detection of a positive signal in a transformed or transfected host cell, as described in PCT publication number WO 95/20652, published Aug. 3, 1995 and is included by reference herein including any figures, tables, or drawings.

Candidate modulators contemplated by the invention include compounds selected from libraries of either potential activators or potential inhibitors. There are a number of different libraries used for the identification of small molecule modulators, including (1) chemical libraries, (2) natural product libraries, and (3) combinatorial libraries comprised of random peptides, oligonucleotides or organic molecules. Chemical libraries consist of random chemical structures, some of which are analogs of known compounds or analogs of compounds that have been identified as “hits” or “leads” in other drug discovery screens, while others are derived from natural products, and still others arise from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Still other candidate inhibitors contemplated by the invention can be designed and include soluble forms of binding partners, as well as such binding partners chimeric, or fusion, proteins. A “binding partner” as used herein broadly encompasses both natural binding partners as described above as well as chimeric polypeptides, peptide modulators other than natural ligands, antibodies, antibody fragments, and modified compounds comprising antibody domains that are immunospecific for the expression product of the identified phosphatase gene.

Other assays may be used to identify specific peptide ligands of a phosphatase polypeptide, including assays that identify ligands of the target protein through measuring direct binding of test ligands to the target protein, as well as assays that identity ligands of target proteins through affinity ultrafiltration with ion spray mass spectroscopy/HPLC methods or other physical and analytical methods. Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domain or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain cannot by itself activate expression of the reporter gene because it does not bind the UAS. However, when both hybrid proteins are present, the noncovalent interaction of the first and second proteins tethers the activation domain to the UAS, activating transcription of the reporter gene. For example, when the first protein is a phosphatase gene product, or fragment thereof, that is known to interact with another protein or nucleic acid, this assay can be used to detect agents that interfere with the binding interaction. Expression of the reporter gene is monitored as different test agents are added to the system. The presence of an inhibitory agent results in lack of a reporter signal.

When the function of the phosphatase polypeptide gene product is unknown and no ligands are known to bind the gene product, the yeast two-hybrid assay can also be used to identify proteins that bind to the gene product. In an assay to identify proteins that bind to a phosphatase polypeptide, or fragment thereof, a fusion polynucleotide encoding both a phosphatase polypeptide (or fragment) and a UAS binding domain (i.e., a first protein) may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

Other assays may be used to search for agents that bind to the target protein. One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

In preferred embodiments of the invention, methods of screening for compounds which modulate phosphatase activity comprise contacting test compounds with phosphatase polypeptides and assaying for the presence of a complex between the compound and the phosphatase polypeptide. In such assays, the ligand is typically labelled. After suitable incubation, free ligand is separated from that present in bound form, and the amount of free or uncomplexed label is a measure of the ability of the particular compound to bind to the phosphatase polypeptide.

In another embodiment of the invention, high throughput screening for compounds having suitable binding affinity to phosphatase polypeptides is employed. Briefly, large numbers of different small peptide test compounds are synthesised on a solid substrate. The peptide test compounds are contacted with the phosphatase polypeptide and washed. Bound phosphatase polypeptide is then detected by methods well known in the art. Purified polypeptides of the invention can also be coated directly onto plates for use in the aforementioned drug screening techniques. In addition, non-neutralizing antibodies can be used to capture the protein and immobilize it on the solid support.

Other embodiments of the invention comprise using competitive screening assays in which neutralizing antibodies capable of binding a polypeptide of the invention specifically compete with a test compound for binding to the polypeptide. In this manner, the antibodies can be used to detect the presence of any peptide that shares one or more antigenic determinants with a phosphatase polypeptide. Radiolabeled competitive binding studies are described in A. H. Lin et al. Antimicrobial Agents and Chemotherapy, 1997, vol. 41, no. 10. pp. 2127-2131, the disclosure of which is incorporated herein by reference in its entirety.

In another aspect, the invention provides methods for treating a disease by administering to a patient in need of such treatment a substance that modulates the activity of a phosphatase selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. Preferably the disease is selected from the group consisting of cancers, immune-related diseases and disorders, cardiovascular disease, brain or neuronal-associated diseases, and metabolic disorders. More specifically these diseases include cancer of tissues or hematopoietic origin; central or peripheral nervous system diseases and conditions including migraine, pain sexual dysfunction, mood disorders attention disorders, cognition disorders, hypotension, and hypertension; psychotic and neurological disorders, including anxiety, schizophrenia, manic depression, delirium, dementia severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, including cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

In preferred embodiments, the invention provides methods for treating or preventing a disease or disorder by administering to a patient in need of such treatment a substance that modulates the activity of a phosphatase polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. Preferably the disease is selected from the group consisting of cancers, immune-related diseases and disorders, cardiovascular disease, brain or neuronal-associated diseases, and metabolic disorders. More specifically these diseases include cancer of tissues or hematopoietic origin; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorder, atherosclerosis; ocular disease including glaucoma retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The invention also features methods of treating or preventing a disease or disorder by administering to a patient in need of such treatment a substance that modulates the activity of a phosphatase polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. Preferably the disease is selected from the group consisting of cancers, immune-related diseases and disorders, cardiovascular disease, brain or neuronal-associated diseases, and metabolic disorders. More specifically these diseases include cancer of tissues or hematopoietic origin; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The invention also features methods of treating or preventing a disease or disorder by administering to a patient in need of such treatment a substance that modulates the activity of a phosphatase polypeptide having an amino acid sequence selected from the group consisting those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. Preferably the disease is selected from the group consisting of immune-related diseases and disorders, cardiovascular disease, and cancer. Most preferably, the immune-related diseases and disorders are selected from the group consisting of rheumatoid arthritis, chronic inflammatory bowel disease, chronic inflammatory pelvic disease, multiple sclerosis, at osteoarthritis, psoriasis, atherosclerosis, rhinitis, autoimmunity, and organ transplantation.

Substances useful for treatment of phosphatase-related disorders or diseases preferably show positive results in one or more in vitro assays for an activity corresponding to treatment of the disease or disorder in question (Examples of such assays are provided and referenced herein). Examples of substances that can be screened for favorable activity are provided and referenced below. The substances that modulate the activity of the phosphates preferably include, but are not limited to, antisense oligonucleotides and inhibitors of protein phosphatases, as determined by methods and screens referenced below.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation, or cell survival.

Abnormal cell proliferative conditions include cancers such as fibrotic and mesangial disorder, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of protein phosphatases are associated with the apoptosis pathways. Aberrations in the function of any one of the protein phosphatases could lead to cell immortality or premature cell death.

The term “aberration”, in conjunction with the function of a phosphatase in a signal transduction process, refers to a phosphatase that is over- or under-expressed in an organism, mutated such that its catalytic activity is lower or higher than wild-type protein phosphatase activity, mutated such that it can no longer interact with a natural binding partner, is no longer modified by another protein phosphatase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig, or goat, more preferably a monkey or ape, and most preferably a human.

In another aspect, the invention features methods for detection of a phosphatase polypeptide in a sample as a diagnostic tool for diseases or disorders, wherein the method comprises the steps of: (a) contacting the sample with a nucleic acid probe which hybridizes under hybridization assay conditions to a nucleic acid target region of a phosphatase polypeptide having an amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, arteriosclerosis, autoimmune disorders, organ transplantation, myocardial infarction, cardiomyopathies, stroke, renal failure, oxidative stress-related neurodegenerative disorders, and cancer.

The phosphatase “target region” is the nucleotide base sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, or the corresponding full-length sequences, a functional derivative thereof, or a fragment thereof to which the nucleic acid probe will specifically hybridize. Specific hybridization indicate that in the presence of other nucleic acids the probe only hybridizes detectably with the phosphatase of the invention's target region. Putative target regions can be identified by methods well known in the art consisting of alignment and comparison of the most closely related sequences in the database.

In preferred embodiments the nucleic acid probe hybridizes to a phosphatase target region encoding at least 6, 12, 75, 90, 105, 120, 150, 200, 250, 300 or 350 contiguous amino acids of a sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ D NO:23, and SEQ ID NO:24, or the corresponding full-length amino acid sequence, or a functional derivative thereof. Hybridization conditions should be such that hybridization occurs only with the phosphatase genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined, above.

The diseases for which detection of phosphatase genes in a sample could be diagnostic include diseases in which phosphatase nucleic acid (DNA and/or RNA) is amplified in comparison to normal cells. By “amplification” is meant increased numbers of phosphatase DNA or RNA in a cell compared with normal cells. In normal cells, phosphatases are typically found as single copy genes. In selected diseases, the chromosomal location of the phosphatase genes may be amplified, resulting in multiple copies of the gene, or amplification. Gene amplification can lead to amplification of phosphatase RNA, or phosphatase RNA can be amplified in the absence of phosphatase DNA amplification.

“Amplification” as it refers to RNA can be the detectable presence of phosphatase RNA in cells, since in some normal cells there is no basal expression of phosphatase RNA. In other normal cells, a basal level of expression of phosphatase exists, therefore in these cases amplification is the detection of at least 1-2-fold, and preferably more, phosphatase RNA, compared to the basal level.

The diseases that could be diagnosed by detection of phosphatase nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissue, cells or extracts to be assayed. Methods for preparing nucleic acid extracts of cells are well known in the art and can be readily adapted in order to obtain a sample that is compatible with the method utilized.

In another aspect, the invention features a method for detection of a phosphatase polypeptide in a sample as a diagnostic tool for a disease or disorder, wherein the method comprises: (a) comparing a nucleic acid target region encoding the phosphatase polypeptide in a sample, where the phosphatase polypeptide has an amino acid sequence selected from the group consisting those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ D NO:22, SEQ ID NO:23, and SEQ ID NO:24, or one or more fragments thereof, with a control nucleic acid target region encoding the phosphatase polypeptide, or one or more fragments thereof, and (b) detecting differences in sequence or amount between the target region and the control target region, as an indication of the disease or disorder. Preferably the disease is selected from the group consisting of cancers, immune-related diseases and disorders, cardiovascular disease, brain or neuronal-associated diseases, and metabolic disorders. More specifically these diseases include cancer of tissues or hematopoietic origin; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, Multiple sclerosis, and Amyotrophic lateral sclerosis; viral infections caused by HIV-1, HIV-2 or other viral- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

Method of Use

Partial amino sequences for human protein phosphatases are encoded by nucleic acid sequences set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

These sequences will be used to find the full-length clone of each of the predicted protein phosphatases. These clones will be useful for screening for small molecule compounds that inhibit the catalytic activity of the encoded protein phosphatase with potential utility in treating disorders including cancers of tissues or blood particular those involving breast, colon, lung, prostate, cervical, brain, ovarian, bladder, or kidney; central or peripheral nervous system diseases and conditions including migraine, pain, sexual dysfunction, mood disorders, attention disorders, cognition disorders, hypotension, and hypertension; psychotic and neurological disorders, including anxiety, schizophrenia, manic depression, delirium, dementia, severe mental retardation and dyskinesias, such as Huntington's disease or Tourette's Syndrome; neurodegenerative diseases including Alzheimer's, Parkinson's, multiple sclerosis and Amyotrophic lateral sclerosis; viral infections caused by HIV-1, HIV-2 or other vial- or prion-agents or fungal- or bacterial-organisms; metabolic disorders including Diabetes and obesity and their related syndromes, among others; cardiovascular disorders including reperfusion restenosis, coronary thrombosis, clotting disorders, unregulated cell growth disorders, atherosclerosis; ocular disease including glaucoma, retinopathy, and macular degeneration; inflammatory disorders including rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplant rejection.

The summary of the invention described above is not limiting and other features and advantages of the invention will be apparent from the following detailed description of the invention, and from the claims.

### BRIEF DESCRIPTION OF THE FIGURES

FIGS. 1A-H show the nucleotide sequences for human protein phosphatases (SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12).

FIGS. 2A-2C provide amino acid sequences for the human protein phosphatases encoded by SEQ ID NO: 1-SEQ ID NO:12 (SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, respectively). Some of the sequences encode predicted stop codons within the coding region, indicated by an ‘x.’

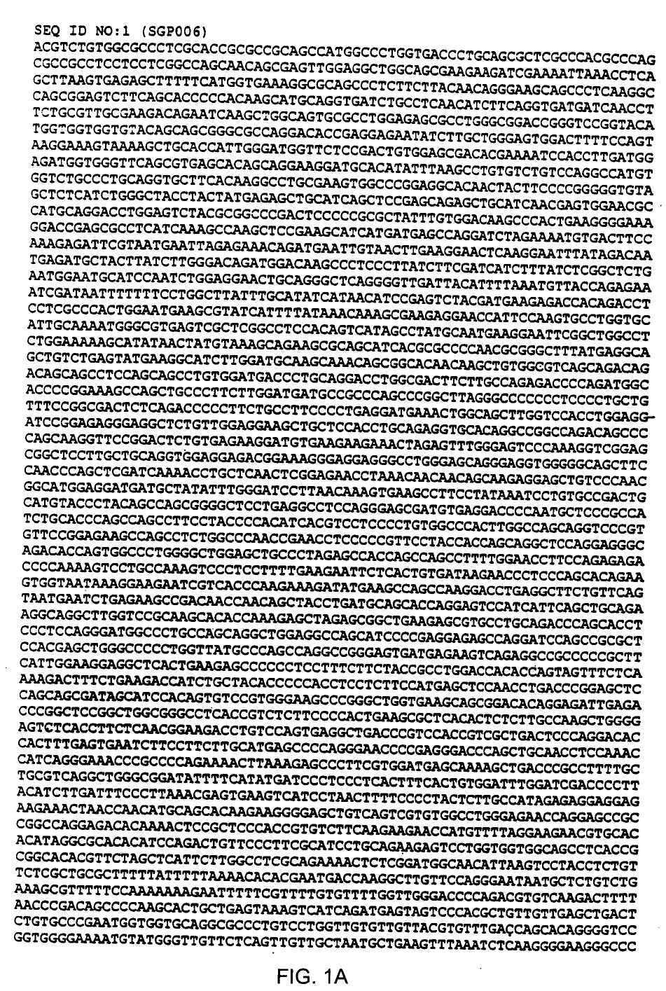

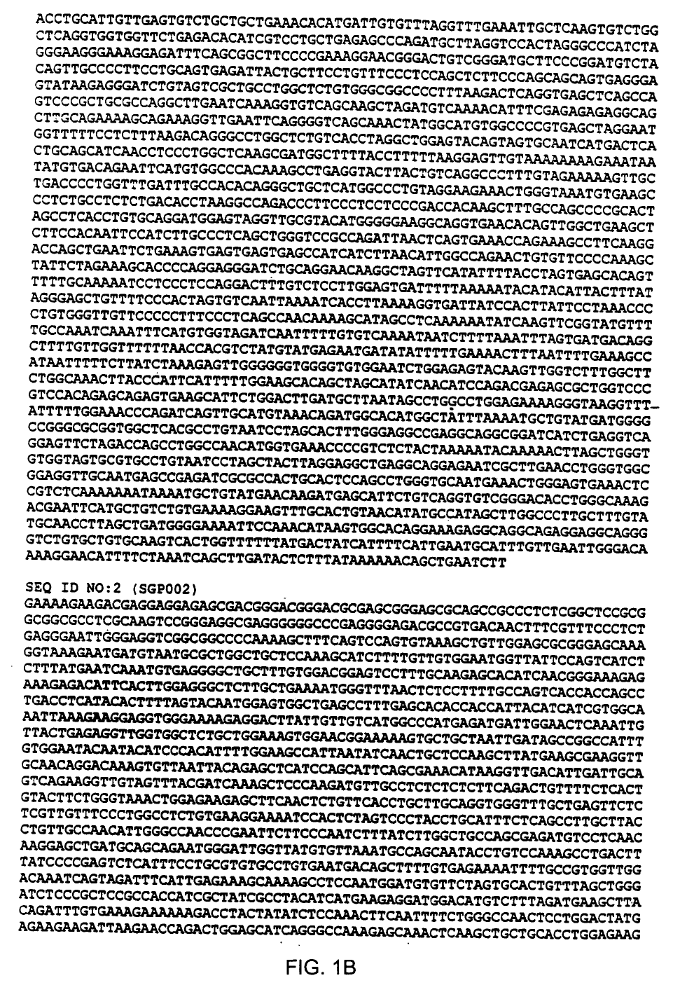

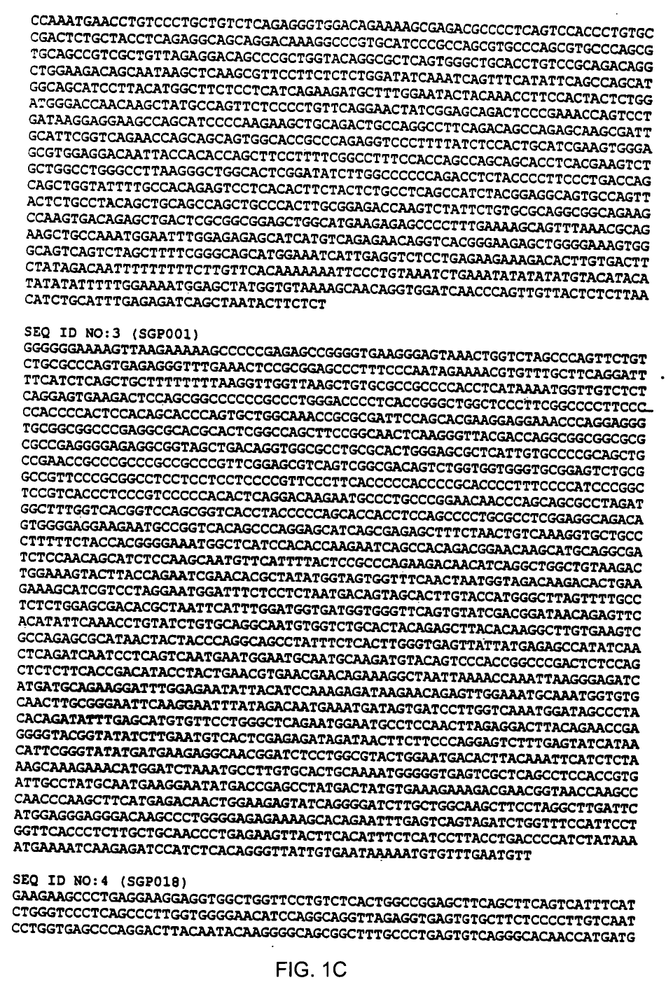

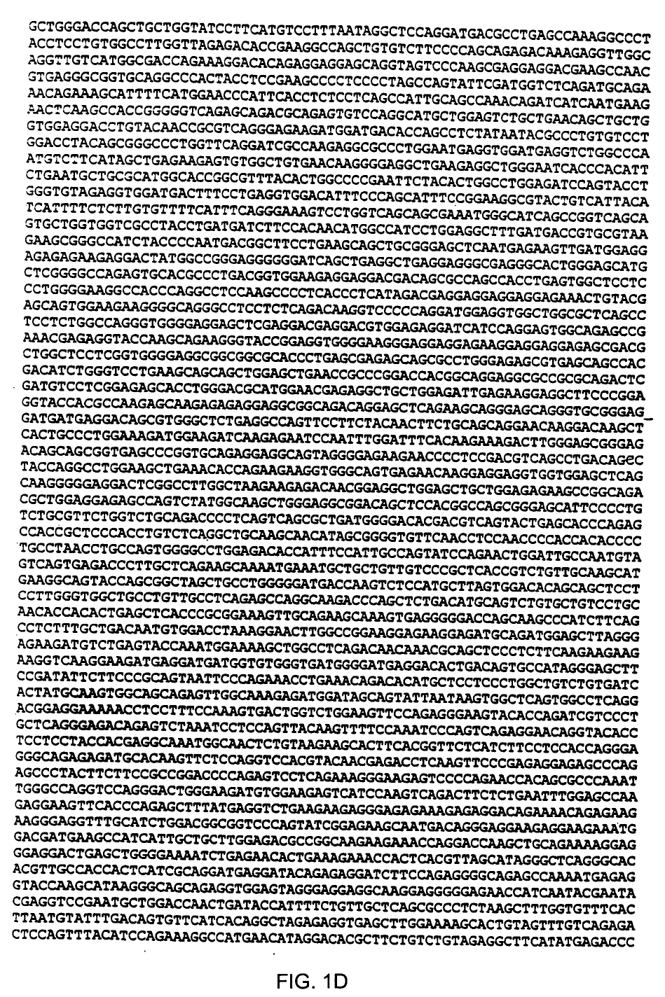

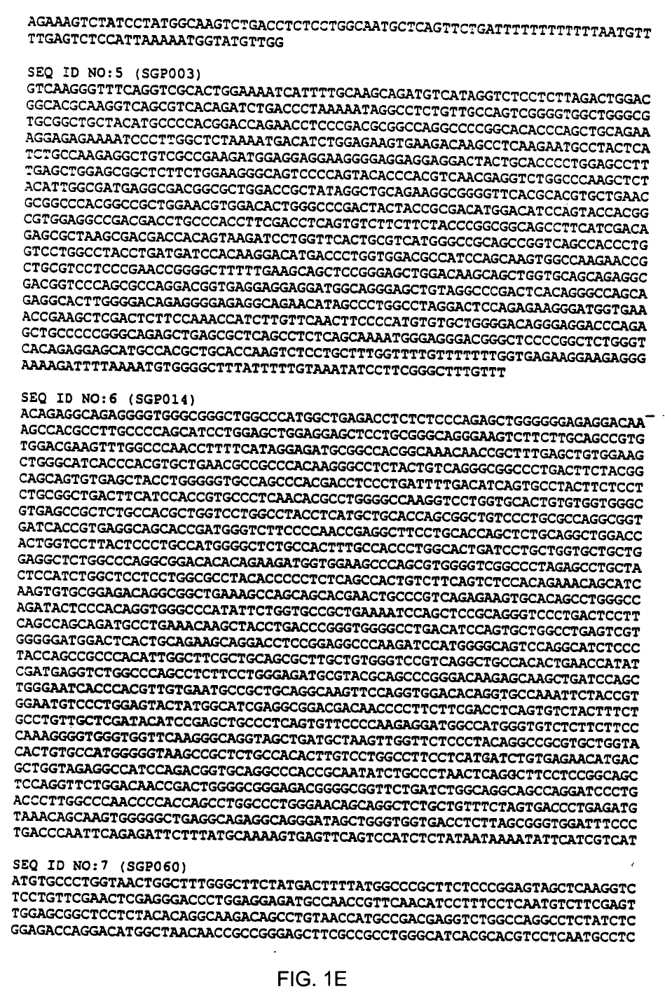

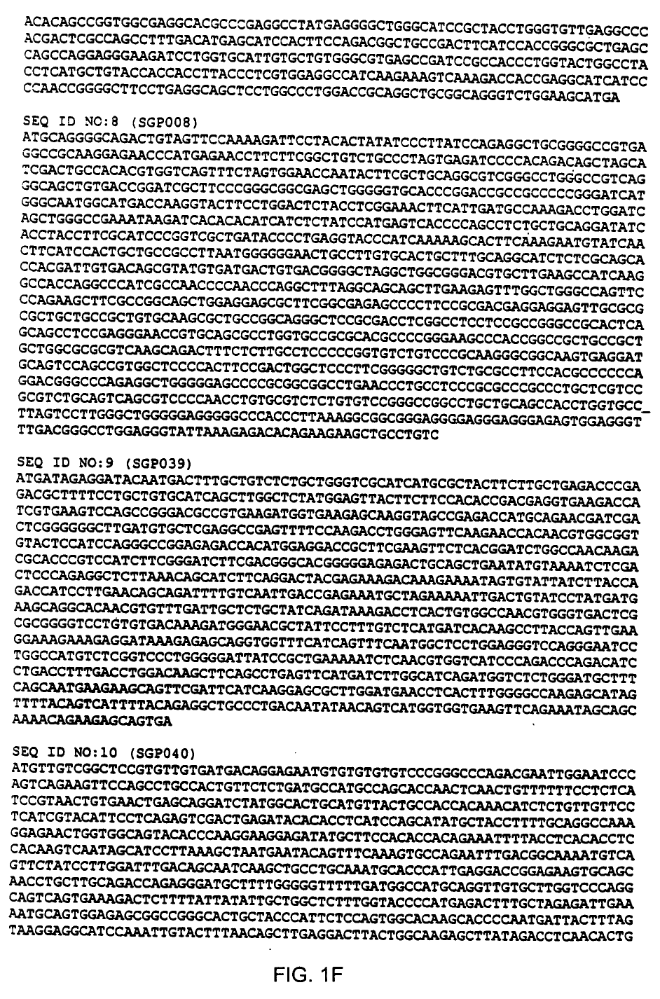

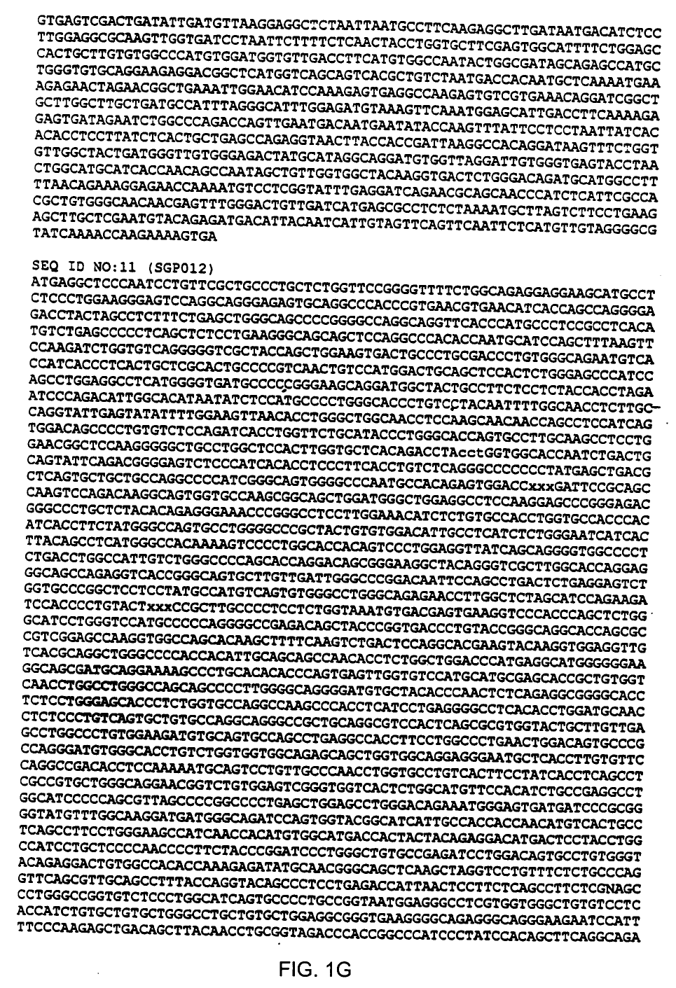

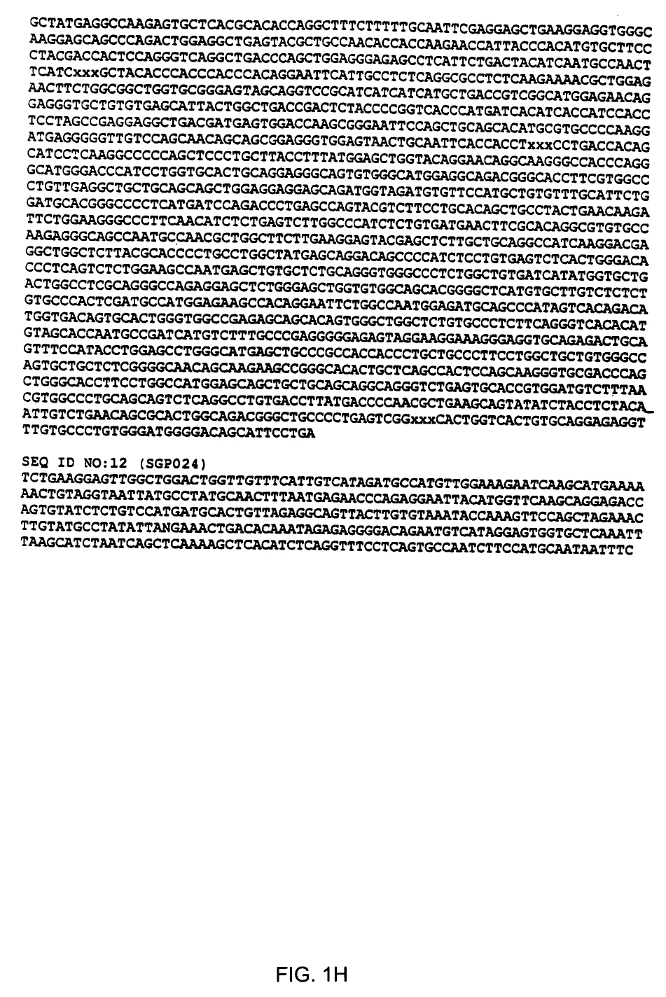

### DETAILED DESCRIPTION OF THE INVENTION

The present invention relates to the isolation and characterization of new polypeptides, nucleotide sequences encoding these polypeptides, various products and assay methods that can be used to identify compounds useful for the diagnosis and treatment of various polypeptide-related diseases and conditions, for example cancer. Polypeptides, preferably phosphatases, and nucleic acids encoding such polypeptides may be produced, using well-known and standard synthesis techniques when given the sequences presented herein. By reference, e.g., to Tables 1 though 8, below, genes according to the invention can be better understood. The invention additionally provides a number of different embodiments, such as those described below.

Nucleic Acids

Associations of chromosomal localizations for mapped genes with amplicons implicated in cancer are based on literature searches (PubMed http://www.ncbi.nlm.nih.gov/entrez/query.fcgi), OMIM searches (Online Mendelian Inheritance in Man, http://www.ncbi.nlm.nih.gov/Omim/searchomim.html) and the comprehensive database of cancer amplicons maintained by Knuutila et al. (Knuutila, et al., DNA copy number amplifications in human neoplasms. Review of comparative genomic hybridization studies. Am J Pathol 152:1107-1123, 1998. http://www.helsinki.fi/˜lgl www/CMG.html). For many of the mapped genes, the cytogenetic region from Knuutila is listed followed by the number of cases with documented amplification and the total number of cases studied. Thus for SGP006 below, the entry “Bladder carcinoma (12q21-q24, 1/16)” means that the chromosomal position has been associated with non-small cell lung cancer, at position 12q21-q24, which encompasses the SGP006's position, and the amplification has been noted in 1 of the 16 samples studied.

For single nucleotide polymorphisms, an accession number (for example, ss1581624 for SGP187) is given if the SNP is documented in dbSNP (the database of single nucleotide polymorphisms) maintained at NCBI (http://www.ncbi.nlm.nih.gov/SNP/index.html). The accession number for SNP can be used to retrieve the full SNP-containing sequence from this site. Candidate SNPs without a dbSNP accession number were identified by inspection of Blastn outputs of the patent sequences vs cDNA and genomic databases, as shown in Table 7 and Table 8, respectively, in Example 1.

Nucleic Acid Probes, Methods, and Kits for Detection of Phosphatases

The present invention additionally provides nucleic acid probes an uses therefor. A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences which correspond to N-terminal and C-terminal portions of the amino acid sequence of the polypeptide of interest. The synthesized nucleic acid probes may be used as printers in a polymerase chain reaction (PCR) carried out in accordance with recognized PCR techniques, essentially according to PCR Protocols, “A Guide to Methods and Applications”, Academic Press, Michael et al., eds., 1990, utilizing the appropriate chromosomal or cDNA library to obtain the fragment of the preset invention.

One skilled in the art can readily design such probes, based on the nucleic acid and amino acid sequences disclosed herein, using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample which is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin). Preferably, the kit further comprises instructions for use.

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats which are well known in the art.

### Categorization of the Polypeptides According to the Invention

For a number of protein phosphatases of the invention, there is provided a classification of the protein class and family to which it belongs, a summary of non-catalytic protein motifs, as well as a chromosomal location. This information is useful in determing function, regulation and/or therapeutic utility for each of the proteins. Amplification of chromosomal region can be associated with various cancers. For amplicons discussed in this application, the source of information was Knuutila, et al (Knuutila S, Björkqvist A-M, Autio K, Tarkkanen M, Wolf M, Monni O, Szymanska J, Larramendy M L, Tapper J, Pere H, El-Rifai W, Hemmer S, Wasenius V-M, Vidgren V & Zhu Y: DNA copy number amplifications in human neoplasms. Review of comparative genomic hybridization studies. Am J Pathol 152:1107-1123, 1998. http://www.helsinki.fi/˜lgl_www/CMG.html).

The phosphatase classification and protein domains often reflect pathways, cellular roles, or mechanisms of up- or down-stream regulation. Also disease-relevant genes often occur in families of related genes. For example, if one member of a phosphatase family functions as an oncogene, a tumor suppressor, or has been found to be disrupted in an immune, neurologic, cardiovascular, or metabolic disorder, frequently other family members may play a related role.

Chromosomal location can identify candidate targets for a tumor amplicon or a tumor-suppressor locus. Summaries of prevalent tumor amplicons are available in the literature, and can identify tumor types to experimentally be confirmed to contain amplified copies of a phosphatase gene which localizes to an adjacent region.

A more specific characterization of the polypeptides of the invention, including potential biological and clinical implications, is provided, e.g., in EXAMPLES 2 and 3.

### Classification of Polypeptides Exhibiting Phosphatase Activity

The polypeptides described in the present invention may belong to one of the following groups: (1) dual-specificity group of protein phosphates (DSP); (2) serine-threonine phosphatases (STP); or (3) protein tyrosine phosphatases (PTP). This classification relies, at least in part on the conserved core amino acid sequence-motifs that make up the catalytic domain of this class of phosphatases.

DSP Group

The unique signature motifs of the catalytic domain of the dual-specificity class of phosphatases is responsible for the ability of these enzymes to dephosphorylate phosphoserine/phosphothreonine as well phosphotyrosine residues. The dual-specificity group of protein phosphatases include the family member MAP kinase phosphatases (MKP). A description of the structural and functional characteristics for the MKP family now follows.

MKP Family

Novel MKP-like phosphatases in this application include SGP006 (SEQ ID NO:1), SGP002 (SEQ ID NO:2), SGP001 (SEQ ID NO:3), SGP018 (SEQ ID NO:4), SGP003 (SEQ ID NO:5), SGP014 (SEQ ID NO:6), SGP060 (SEQ ID NO:7), and SGP008 (SEQ ID NO:8), which are disclosed in greater detail in the Tables 1-6 and Example 2, for example.

The dual specificity phosphatase family includes around 20 known human members (for a list, see http://smart.emblheidelberg.de/smart/get_member.pl?WHAT=species&NAME=DSPc&WHICH=Homo_sapiens ). Well-known members of the MPK family of dual-specificity phosphatases include: DUS1 (also known as MPK-1, CLI100, PTPN-10, erp, VH1 or 3CH134), DUS3 (also known as VHR), DUS4 (also known as HVH2, TYP1, MKP2 or VH2), DUS5 (also known as HVH3, B23, VH3), DUS6 (also known as PYST1, MKP3, rVH6), DUS7 (also known as PYST2), CDKN3 (also known as CDKN3, KAP, CIP2 or CDI1), VH5 and STYX.

Most MKP phosphatases are capable of inactivating, through a dephosphorylation reaction, kinases that participate in the MAPK pathways. The ERK (extracellular signal-regulated kinase), JNK/SAPK (c-Jun N-terminal kinase/stress-activated protein kinase) and p38 MAP kinase pathways mediate the signal transduction events that are responsible for cell division, differentiation or apoptosis in response to extracellular ligands (Cobb M H, Prog Biophys Mol Biol. 1999;71(3-4):479-500). Full MAP kinase enzymatic activation requires the concomitant phosphorylation by selective upstream dual-specificity kinases of threonine and tyrosine residues residing in the activation loop of the MAP kinases. MKP family dual-specificity phosphatases mediate MAP kinase inactivation by dephosphorylating these threonine and tyrosine residues. This mechanism provides negative feedback regulation of the MAP kinase pathways. MKPs may play a significant role in human cancer by attenuating MAP kinase cascades involved in cellular transformation.

Given the large number of MAP kinases, as well as MKP's, a central question is whether there is selectivity in kinase substrate recognition by MKP's. Evidence that such specificity exists provided by DUS-6 (MKP3) and VH5 which have been shown to be highly selective phosphatases towards the ERK or JNK/SAPK and p38 MAP kinases, respectively (Muda M, et al., J Biol Chem. 1996 Nov. 1;271(44):27205-8.). Another level of substrate specificity comes from subcellular compartmentalization as shown by DUS-6 (MKP3) which is found exclusively in the cytosol rather in the nucleus (Groom, L. A. et al (1996) EMBO J. 15: 3621-3632). Further specificity can arise at the level of the tissue specificity of expression (i.e. Muda, M. et al (1997) J. Biol Chem 272:5141-5151).

MKP's appear to be as ubiquitous in their phylogenetic distribution as their MAP kinase counterparts with multiple members present in yeast (i.e. YVH1), C. elegans (i.e. Y042), Drosophila, (i.e. puckered), plants (i.e. DsPTP1) and mammals. The primary mode of action of MKP's isolated from different species appears to be MAPK dephosphorylation thereby providing negative feedback to the MAPK signal transduction pathways.

MKP's may play an important role during pathophysiological hypoxia as suggested by the induction of MKP-1 gene expression under low oxygen conditions (Laderroute, K. R. (1999) J. Biol. Chem. 274:12890-12897). Tumor hypoxia is directly linked to the onset of angiogenesis during malignant progression (Hanahan, D. et al (1996) Cell 86:353-364 and Mazure, N. M. et al (1996) Cancer Res. 56:3436-3440). A number of genes have been found to be induced during hypoxic conditions such as the heat shock transcription factor-1 (HSF-1) (Benjamin I. J. et a. (1990) Proc. Natl. Acad. Sci. 87:6263-6267), c-fos and c-jun (Ausserer, W. A. et al (1994) Mol. Cell. Biol. 14:5032-5042, and Muller, J. M. (1997) J. Biol. Chem 272:23435-23439) and the hypoxia-inducible factor-1 (HIF-1) (Wenger, R. H. et al (1997) J. Biol. Chem. 378:609-616). MKP-1 transcripts and protein have been shown to be upregulated in early-stage carcinomas well as in multiple stages of breast and prostate carcinomas (i.e. Leav, I. Et al (1996) Lab. Invest. 75: 361-370). Over-expression of MKP-1 in prostate tumor cell lines confers resistance to Fas ligand-induced apoptosis (Srikanth, S. et al. (1999) Mol. Cell. Biochem. 199:169-178) and it has also been suggested that MKP-1 may contribute to the inhibition of apoptosis resulting in androgen-independent growth. MKP-1 may also inhibit the induction of apoptosis that is produced by anti-neoplastic agents such as cisplatin and camptothecin (Sanchez-Perez, I et al. (2000) Oncogene 19: 5142-5152; Costa-Pereira, A. P. et al. (2000) Br. J. Cancer 82: 1827-1834). Since hypoxic conditions are known to trigger apoptosis via the activation of the JNK pathway (reviewed in Ip, Y. T. et al (1998) Curr. Opin. Cell Biol. 10:205-219) and MAPK phosphatases provide negative feedback to this pathway, it is conceivable that MKP-1 supports tumor growth by blocking apoptosis. Over-expression of MKP-1 can block the hypoxia-induced activation of SAPK/JNK in co-transfected tumor cells (Laderroute, K. R. (1999) J. Biol. Chem. 274:12890-12897).

The dephosphorylation and subsequent inactivation of ERK-1 and ERK-2 by MAPK phosphatases may also be responsible for suppressing angiogenic vascular endothelial cell proliferation by angiostatin Redlitz A. et al. (1999 J. Vasc. Res 36:28-34).

The novel MPK family phosphatases presented in this filing contribute to a growing list of phosphatases that appear to have as their primary function negative feedback regulation of MAPK signal transduction. Since there is precedence for selectivity in the mechanism of action at the level of substrate recognition, subcellular localization and tissue distribution among the known MPK's, the novel MPK's described may display similar selectivity. The novel MPK's may also play a role in suppressing apoptosis by blocking the JNK/SAPK pathway during pathological hypoxia such as that occurring in angiogenic tumors. The development of specific phosphatase inhibitors that target the anti-apoptotic MKP's may prove valuable as an approach to cancer therapy.

PTP Group

There are 2 PTP-like sequences in this application: SGP012 (SEQ ID NO:11) and SGP024 (SEQ ID NO:12), which are disclosed in greater detail in the Tables 1-6 and Example 2, for example.

SGP012 is closely related to murine OST-PTP, also called PTP-ESP. Osteotesticular PTP (OST-PTP) is a putative receptor protein tyrosine phosphatase that possesses 10 fibronectin type III repeats, a potential membrane-spanning region and an intracellular domain consisting of two tandem catalytic domains. The expression pattern is highly restricted and is detectable primarily in bone and testis (Mauro et al. J Biol Chem 1996 269:30659-67). The ligand for OST-PTP is not known but the structure of the extracellular domain suggests that cell-cell interactions may be involved. Importantly, the human ortholog has not yet been cloned.

The balance between bone deposition and resorption is controlled by the relative activities of two cell types, osteoblasts and osteoclasts. The potential role of phosphatases in bone metabolism is only incompletely understood. However, in osteoblast cultures, inhibition of PTP activity with orthovanadate enhances matrix formation (Lau et al. Endocrinology 188 123:2858-67). In addition, bisphophonates, which are used clinically to treat bone diseases with excess resorption, cause a range of changes in osteoblast cultures that are consistent with increased bone deposition including osteoblast differentiation, alkaline phosphatase activity, type I collagen secretion, and mineralization (Reinholz et al. Cancer Research 2000 60:6001-007). The molecular target of these compounds is still unknown, but it is plausible that inhibition of OST-PTP activity is responsible for the observed increases in bone-forming activities in osteoblast cultures. Therefore targeting of OST-PTP activity could provide treatments for osteoporosis, non-healing fractures, and other disorders of bone metabolism.

SGP024 represents a partial PTPT catalytic domain related to PTP-delta.

STP Group

The are 2 STP proteins in this application: SGP039 (SEQ ID NO:9) and SGP040 (SEQ ID NO:10), which are disclosed in greater detail in the Tables 1-6 and Example 2, for example.

The Serine-threonine phosphatases can be divided into four major classes represented by PP1, PP2A, PP2B, and PP2C. PP2a is found associated with multiple regulatory subunits and its inactivation leads to transformation by viral components such as small T antigen. Mutations in one of the regulatory subunits have been associated with colorectal cancers consistent with a role as a tumor suppressor (Takagi et al. Gut 2000 47:268-71. Recently, PP2a has also been implicated in activation of T lymphocytes (Chuang et al. Immunity 2000 13:313-22). PP1 has been implicated in a variety of cellular functions including response to hypoxia, apoptosis and cytokinesis (Taylor et al., PNAS 2000 97:12091-96, Aylion et a. EMBO J 2000 19 2237-46. Orr et al., Infect. Immun. 2000 68:1350-58). Finally, studies in diabetic rats showed decreased PP1 activity and elevated PP2A activity compared to controls (Begum and Ragolia Metabolism 1998 47:54-62). Because of the diversity of regulatory subunits that affect the activity of serine-threonine phosphatases, biological function of novel members are difficult to predict. However, the studies suggest potential involvement in a variety of diseases including tumorigenesis, inflammatory diseases, and metabolic diseases.

### Therapeutic Methods According to the Invention

Diagnostics:

The invention provides methods for detecting a polypeptide in a sample as a diagnostic tool for diseases or disorders, wherein the method comprises the steps of: (a) contacting the sample with a nucleic acid probe which hybridizes under hybridization assay conditions to a nucleic acid target region of a polypeptide selected from the group consisting of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, atherosclerosis, autoimmune disorders, organ transplantation, myocardial infarction, cardiomyopathies, stroke, renal failure, oxidative stress-related neurodegenerative disorders, metabolic disorder including diabetes, reproductive disorders including infertility, and cancer.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined herein.

The diseases for which detection of genes in a sample could be diagnostic include diseases in which nucleic acid (DNA and/or RNA) is amplified in comparison to normal cells. By “amplification” is meant increased numbers of DNA or RNA in a cell compared with normal cells.

“Amplification” as it refers to RNA can be the detectable presence of RNA in cells, since in some normal cells there is no basal expression of RNA. In other normal cells, a basal level of expression exists, therefore in these cases amplification is the detection of at least 1-2-fold, and preferably more, compared to the basal level.

The diseases that could be diagnosed by detection of nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Antibodies, Hybridomas, Methods of Use and Kits for Detection Phosphatases:

The present invention relates to an antibody having binding affinity to a phosphatase of the invention. The polypeptide may have the amino acid sequence selected from the group consisting of those set forth in SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, or a functional derivative thereof or at least 9 contiguous amino acids thereof (preferably, at least 20, 30, 35, or 40 contiguous amino acids thereof).

The present invention also relates to an antibody having specific binding affinity to a phosphatase of the invention. Such an antibody may be isolated by comparing its binding affinity to a phosphatase of the invention with its binding affinity to other polypeptides. Those which bind selectively to a phosphatase of the invention would be chosen for use in methods requiring a distinction between a phosphatase of the invention and other polypeptides. Such methods could include, but should not be limited to, the analysis of altered phosphatase expression in tissue containing other polypeptides.

The phosphatases of the present invention can be used in a variety of procedures and methods, such as for the generation of antibodies, for use in identifying pharmaceutical compositions, and for studying DNA/protein interaction.

The phosphatases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the preset invention may be generated using one of the procedures known in the art such as chimerization or CDR grafting.

The present invention also relates to a hybridoma which produces the above-described monoclonal antibody, or binding fragment thereof. A hybridoma is an immortalized cell line which is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal which is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globule or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell which produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al, J. Histochem. Cytochem. 18:315, 1970; Bayer et al., Meth. Enzym. 62:308, 1979; Engval et al., Immunol. 109:129, 1972; Goding, J. Immunol. Meth. 13:215, 1976. The labeled antibodies of the present invention can be used for in vitro, in vivo, and in situ assays to identify cells or tissues which express a specific peptide.

The above-described antibodies may also be immobilized on a solid support. Examples of such solid supports include plastics such as polycarbonate, complex carbohydrates such as agarose and sepharose, acrylic resins such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilize antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, NY, pp. 289-307, 1992; Kaspczak et al., Biochemistry 28:9230-9238, 1989).

Anti-peptide peptides can be generated by replacing the basic amino acid residues found in the peptide sequences of the phosphatases of the invention with acidic residues, while maintaining hydrophobic and uncharged polar groups. For example, lysine, arginine, and/or histidine residues are replaced with aspartic acid or glutamic acid and glutamic acid residues are replaced by lysine, arginine or histidine.

The present invention also encompasses a method of detecting a phosphatase polypeptide in a sample, comprising (a) contacting the sample with an above-described antibody, under conditions such that immunocomplexes form, and (b) detecting the presence of said antibody bound to the polypeptide. In detail the methods comprise incubating a test sample with one or more of the antibodies of the present invention and assaying whether the antibody binds to the test sample. Altered levels of a phosphatase of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion-based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry,” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology,” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents which are capable of reacting with the labeled antibody. The compartmentalized kit may be as described above for nucleic acid probe kits. One skilled in the art will readily recognize that the antibodies described in the present invention can readily be incorporated into one of the established kit formats which are well known in the art.

Isolation of Compounds Which Interact with Phosphatases

The present invention also relates to a method of detecting a compound capable of binding to a phosphatase of the invention comprising incubating the compound with a phosphatase of the invention and detecting the presence of the compound bound to the phosphatase. The compound may be present within a complex mixture, for example, serum, body fluid, or cell extracts.

The present invention also relates to a method of detecting an agonist or antagonist of phosphatase activity or phosphatase binding partner activity comprising incubating cells that produce a phosphatase of the invention in the presence of a compound and detecting changes in the level of phosphatase activity or phosphatase binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

Modulating Polypeptide Activity:

The invention additionally provides methods for treating a disease or abnormal condition by administering to a patient in need of such treatment a substance that modulates the activity of a polypeptide selected from the group consisting of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, a functional derivative thereof, and a fragment thereof. Preferably, the disease is selected from the group consisting of rheumatoid arthritis, atherosclerosis, autoimmune disorders, organ transplantation, myocardial infarction, cardiomyopathies, stroke, renal failure; oxidative stress-related neurodegenerative disorders, metabolic and reproductive disorders, and cancer.

Substances useful for treatment of disorders or diseases preferably show positive results in one or more assays for an activity corresponding to treatment of the disease or disorder in question Substances that modulate the activity of the polypeptides preferably include, but are not limited to, antisense oligonucleotides and inhibitors of protein phosphatases.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (, slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation or cell survival. An abnormal condition may also include irregularities in cell cycle progression, i.e., irregularities in normal cell cycle progression through mitosis and meiosis.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to, neurodegenerative disorders, slow wound healing rates, and slow tissue grafting healing rates.

Abnormal cell survival conditions may also relate to conditions in which programmed cell death (apoptosis) pathways are activated or abrogated. A number of protein phosphatases are associated with the apoptosis pathways. Aberrations in the function of any one of the protein phosphatases could lead to cell immortality or premature cell death.

The term “aberration”, in conjunction with the function of a phosphatase in a signal transduction process, refers to a phosphatase that is over- or under-expressed in an organism, mutated such that its catalytic activity is lower or higher than wild-type protein phosphatase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral, parenteral, dermal, injection, and aerosol applications. For cells outside of the organism, multiple techniques exist in the art to administer the compounds, including (but not limited to) cell microinjection techniques, transformation techniques and carrier techniques.

The abnormal condition can also be prevented or treated by administering a compound to a group of cells having an aberration in a signal transduction pathway to an organism. The effect of administering a compound on organism function can then be monitored. The organism is preferably a mouse, rat, rabbit, guinea pig or goat, more preferably a monkey or ape, and most preferably a human.

Stimulating or Antagonizing Phosphatase-Associated Activity

The present invention also encompasses a method of agonizing (stimulating) or antagonizing phosphatase associated activity in a mammal comprising administering to said mammal an agonist or antagonist to an amino acid sequence selected from the group consisting of SEQ ID NO:13, SEQ ID NO:14, SEQ ID NO:15, SEQ ID NO:16, SEQ ID NO:17, SEQ ID NO:11, SEQ ID NO:19, SEQ ID NO:20, SEQ ID NO:21, SEQ ID NO:22, SEQ ID NO:23, SEQ ID NO:24, a functional derivative thereof, and a fragment thereof in an amount sufficient to effect said agonism or antagonism. The present application also contemplates a method of treating diseases in a mammal with an agonist or antagonist of the activity of one of the above mentioned polypeptides of the invention comprising administering the agonist or antagonist to a mammal in an amount sufficient to agonize or antagonize a phosphatase-associated function.

The relevance of a phosphatase gene to a particular diseased condition can be evaluated in order to effect treatment. According to one embodiment of the present invention, microarray expression analysis is performed to establish expression profiles of various phosphatase genes according to the invention, and thereby identify the ones whose expression correlates with certain diseased conditions.

Due to the broad functional implications of various phosphatase families, such treatment may be effectuated to a wide range of diseases, including cancer, pathophysiological hypoxia, cardiovascular disorders, Papillon-Lefevre syndrome, Cowden disease, ectordermal dysplasia, Moebius syndrome, Bjornstad syndrome, Bannayan Zonana syndrome, schizophrenia and hamartomas. Of particular importance is treatment to various type of cancers. Accordingly, the present invention provides methods for treating pathologies, including breast cancer, urogenital cancer, prostate cancer, head and neck cancer, lung cancer, synovial sarcomas, renal cell carcinoma, non-small cell lung cancer, hepatocellular carcinoma, pancreatic endocrine tumors, stomach cancer, gliobastoma, colorectal cancer, and thyroid cancer.

For example, cDNAs made from RNA samples of a variety of tissue sources were spotted onto nylon membranes and hybridized with radiolabeled probes derived from the phosphatase genes of interest. Referring to Example 3 and table 5, phosphatase gene sequences used include: SEQ ID NO4, SEQ ID NO:5, and SEQ ID NO:7. As discussed in the description of Table 5, infra, samples from normal tissues, tumor tissues, various cell lines, and P53 wild type and mutant were used to make the expression array. As shown in Example 3, the relative gene expression levels of the tested phosphatase genes in various tissue sources and cell lines were quantitated by measuring Syber Green I staining of hybridized signals. The numerical readings recorded in the table were normalized to the hybridization result from ds cDNA or undenatured probes, after subtracting the background counts.

Together with the information of corresponding nucleic acid and amino acid sequences provided herein, the relevant expression levels in Table 5 constitutes expression profiles of the phosphatase genes of interest in various tissue sources. Such expression profile data guides application of the treatment regime according to the present invention. For example, referring to the sample, “M14” cell line (a malignant melanoma) in Table 5, the levels of expression of SEQ ID NO:4 is zero. The level of expression of SEQ ID NO:7 (58) is low to marginal. However, the level of expression of SEQ ID NO:5 (2,528) is significantly higher. Such horizontal comparison reveals that the phosphatase gene encoded by SEQ ID NO:5 is implicated in melanoma. That is, manipulation of the function activities of this gene may affect the cancerous condition of malignant melanoma SEQ ID NO:5 (SGP003) encodes SEQ ID NO:17, a protein belonging to the MKP family, as shown in Table 1, for example. Therefore, a method of treating the cancer condition connected to a malignant melanoma can be, for example, to administer to the patient suffering from this cancer an agent that is capable of modulating the activities of the phosphatase activity of the protein represented by SEQ ID NO:17. The expression analysis according to the preferred embodiment of this invention, thus, confers specificity and effectiveness to the method of treatment disclosed.

It should be appreciated that many ways of comparison and correlation analysis may be carried out, based on expression data generated in the way similar to that described in Example 3. These ways will be apparent to one skilled in the art, based on the above discussion and, therefore, fall within the scope of the invention. Inferences derived from those comparison and correlation analysis similarly may be used in substantiating a treatment method or regimen, according to the invention. For instance, when pairs of samples of normal tissues and diseased tissues are used to make the expression arrays, the data generated will specifically demonstrate which phosphatase genes are differentially expressed in certain diseased conditions and, thereby, form targets of the treatment method according to the present invention. That is, modulators or agents that are capable of regulating their activities, either in vivo or in vitro, may be identified and used in the treatment of the given diseased conditions.

According to the present invention, there also is provided a method for detecting a phosphatase in a sample as a diagnostic tool for a disease or disorder using nucleotide probes derived from the phosphatase gene sequences disclosed in the present invention, such as those disclosed herein. Due to the broad functional implications of various phosphatase families, such diagnostic measures may be used for a wide range of diseases, including cancer, pathophysiological hypoxia, cardiovascular disorders, Papillon-Lefevre syndrome, Cowden disease, ectordermal dysplasia, Moebius syndrome, Bjornstad syndrome, Bannayan Zonana syndrome, schizophrenia and hamartomas. Of particular importance is diagnose of various type of cancers. The diagnostic method of the present invention may be used to test for breast cancer, urogenital cancer, prostate cancer, head and neck cancer, lung cancer, synovial sarcomas, renal cell carcinoma, non-small cell lung cancer, hepatocellular carcinoma, pancreatic endocrine tumors, stomach cancer, gliobastoma, colorectal cancer, and thyroid cancer.

In a similar vein, it is useful to determine the level of relevance of a phosphatase gene to a particular diseased condition in order to effect accurate diagnoses. Such determinations can be accomplished by performing microarray expression analysis according to one embodiment of this invention. The phosphatase genes whose expression correlates with certain diseased conditions may be identified by the procedure described above.

The data obtained from the microarray data also can be used to diagnose a patient who may be suffering from a particular pathology. A method of diagnosing the cancer condition connected to melanoma, according to the present invention is, therefore, to contact a test sample, which may be collected from a patient, with a nucleotide probe which is capable of hybridizing to the nucleic acid sequence which encodes the protein represented by SEQ ID NO:17; and then to detect the presence of the hybridized probe:target pairs and to quantify the level of such hybridization as an indication of the cancer condition connected to neuroblastoma The expression analysis according to the preferred embodiment of this invention, thus, confers specificity and effectiveness to the diagnostic method disclosed.

As discussed above, many ways of comparison and correlation analysis may be carried out based on expression data generated in the way similar to that described here; they also necessarily fall in the scope of the present invention. Inferences derived from those comparison and correlation analysis may similarly be used in substantiating the diagnostic method according to this invention. One scenario to be noted is when pairs of samples of normal tissues and diseased tissues are used to make the expression arrays, the data generated will specifically demonstrate which phosphatase genes are differentially expressed in certain diseased conditions, therefore may serve as diagnostic markers used in the aforementioned diagnostic method.

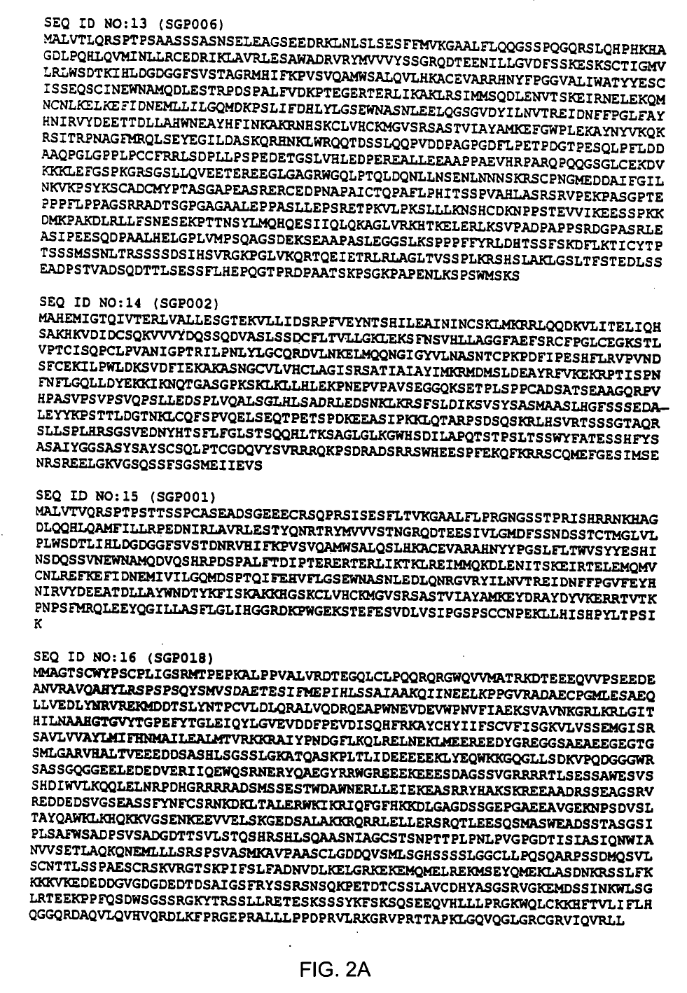

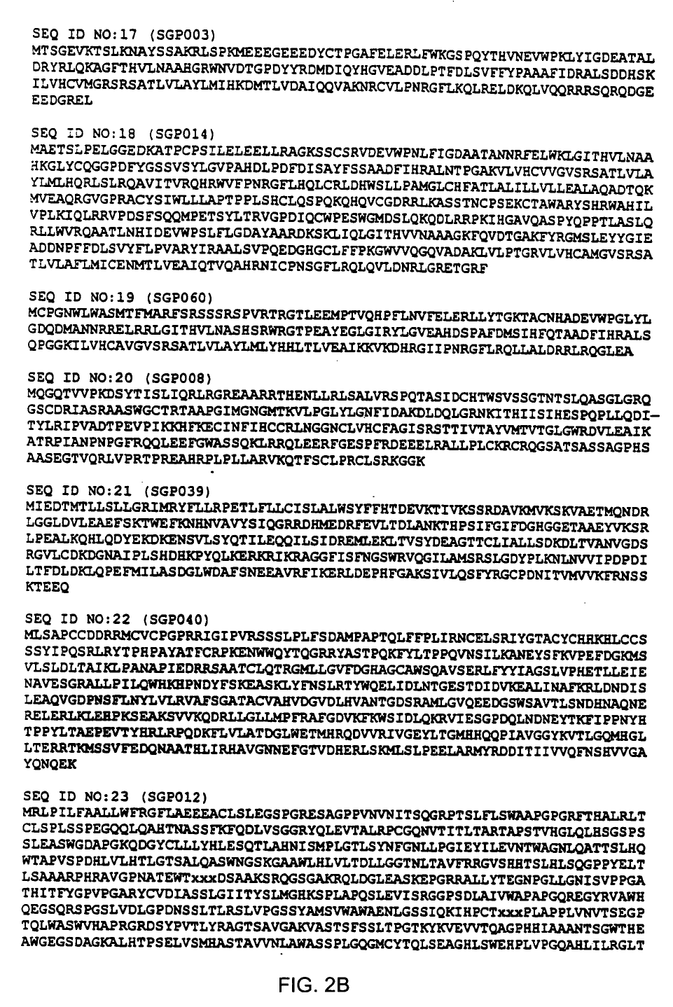

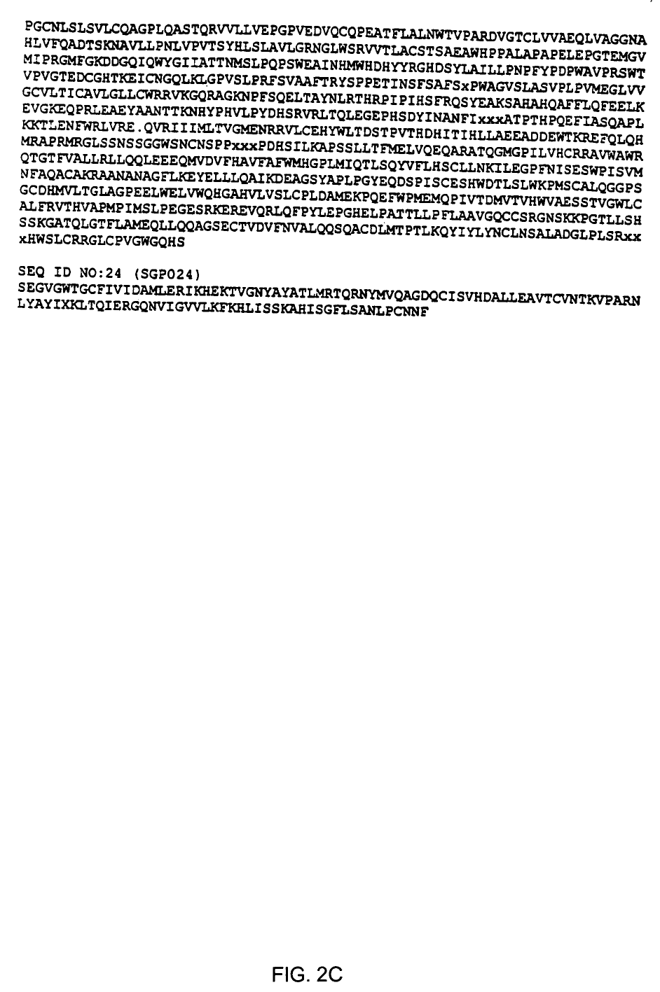

According to the present invention, there also is provided another method for detection of a phosphatase in a sample as a diagnostic tool for a disease or disorder by comparing a nucleic acid target region of the phosphatase genes disclosed in the present invention, such genes encoding the amino acid sequences listed in FIG. 2, with a control region; and then detecting differences in sequence or amount between the target region and control region as an indication of the disease or disorder. This method also may be used for diagnosing a wide range of diseases, including cancer, pathophysiological hypoxia, cardiovascular disorders, Papillon-Lefevre syndrome, Cowden disease, ectordermal dysplasia, Moebius syndrome, Bjornstad syndrome, Bannayan Zonana syndrome, schizophrenia and hamartomas. Of particular importance is diagnosis of various type of cancers. As the aforementioned diagnostic method, this particular method may similarly be used to test for breast cancer, urogenital cancer, prostate cancer, head and neck cancer, lung cancer, synovial sarcomas, renal cell carcinoma, non-small cell lung cancer, hepatocellular carcinoma, pancreatic endocrine tumors, stomach cancer, gliobastoma, colorectal cancer, and thyroid cancer.

A target region can be any particular region of interest in a phosphatase gene, such as an upstream regulatory region. Variations of sequence in an upstream regulatory region in a family of phosphatase often have functional implications some of which may be significant in bringing about certain diseased conditions. Changes of the amount of a target region, e.g., changes of number of copies of a regulatory region such as a receptor-binding site, in certain phosphatase genes, may also represent mechanisms of functional differentiation and hence may be connected to certain diseased states. Detection of such differences in sequence and amount of a target region compared to a control region therefore may effectively lead to detection of a diseased condition.

In one embodiment of the present invention, microarray studies may be used to identify the potential connections between a diseased condition and variations of a target region among a set of phosphatase genes. For example, nucleic acid probes may be made that correspond to a given target region and a control region, respectively, of a phosphatase gene of interest. Samples from normal and diseased tissues are used to make microarray as discussed, supra, and in Example 3. Hybridization of these probes to the array so made will yield comparative profiles of the region of interest in the normal and diseased condition, and thus may derive a definition of differences of the target region and control region that is characterized of the disease in question. Such definition, in turn, may serve as an indication of the diseased condition as used in the second-mentioned diagnostic method according to the present invention. It should be appreciated that many equivalent or similar methods may be used in carrying out the diagnosis according to this method which would become apparent to the skilled person in the art based on the example provided here, and therefore, they are covered in the scope of this invention.

In an effort to discover novel treatments for diseases, biomedical researchers and chemists have designed, synthesized, and tested molecules that inhibit the function of protein phosphatases. Some small organic molecules form a class of compounds that modulate the function of protein phosphatases. Examples of molecules that have been reported to inhibit the function of protein phosphatases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compound (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein phosphatase inhibitors only weakly inhibit the function of protein phosphatases. In addition, many inhibit a variety of protein phosphatases and will therefore cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar moieties including hydroxylated alkyl phosphate, and ether moieties. U.S. patent application Ser. No. 08/702,232, filed Aug. 23, 1996, entitled “Idolinone Combinatorial Libraries and Related Products and Methods for the Treatment of Disease” by Tang et al. (U.S. Ser. No. 08/702,232) and U.S. Pat. No. 5,880,141, entitled “Benzylidene-Z-Indoline Compounds for the Treatment of Disease” by Tang et al. (U.S. Ser. No. 08/485,323) and International Patent Publications WO 96/40116, published Dec. 19, 1996 by Tang, et al, and WO 96/22976, published Aug. 1, 1996 by Ballinari et al, all of which are incorporated herein by reference in their entirety, including any drawing, figures, or tables, describe indolinone chemical libraries of indolinone compounds harboring other bicyclic moieties as well as monocyclic moieties fused to the oxindole ring. Application Ser. No. 08/702,232, filed Aug. 23, 1996, entitled “Indolinone Combinatorial Libraries and Related Products and Methods for the Treatment of Disease” by Tang et al.; U.S. Pat. No. 5,880,141, filed Jun. 7, 1995, entitled “Benzylidene-Z-Indoline Compounds for the Treatment of Disease” by Tang et al. (U.S. Ser. No. 08/485,323), and WO 96/22976, published Aug. 1, 1996 by Ballinari et al. teach methods of indolinone synthesis, methods of testing the biological activity of indolinone compounds in cells, and inhibition patterns of indolinone derivatives.

Other examples of substances capable of modulating phosphatase activity include, but are not limited to, tyrphostins, quinazolines, quinoxolines, and quinolines. The quinazolines, tyrphostins, quinzolines, and quinoxolines referred to above include well known compounds such as those described in the literature. For example, representative publications describing quinazolines include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5,316,553; Kreighbaum and Comer, U.S. Pat. No. 4,343,940; Pegg and Wardleworth, EPO Publication No. 0 562 734 A1; Barker et al., (1991) Proc. of Am. Assoc. for Cancer Research 32:327; Bertino, J. R., (1979) Cancer Research 3:293-304; Berino, J. R., (1979) Cancer Research 9(2 part 1):293-304; Curtin et al., (1986) Br. J. Cancer 53:361-368;Fernandes et al., (1983) Cancer Research 43:1117-1123; Ferris et al. J. Org. Chem. 44(2):173-178; Fry et al., (1994) Science 265:1093-1095; Jackman et al., (1981) Cancer Research 51:5579-5586; Jones et al. J. Med Chem 29(6):1114-1118; Lee and Skibo, (1987) Biochemistry 26(23):7355-7362; Lemus et al., (1989) J. Org. Chem. 54:3511-3518; Ley and Seng, (1975) Synthesis 1975:415-522; Maxwell et al., (1991) Magnetic Resonance in Medicine 17:189-196; Mini et al., (1985) Cancer Research 45:325-330; Phillips and Castle, J. (1980) Heterocyclic Chem. 17(19):1489-1596; Reece et al., (1977) Cancer Research 47(11):2996-2999; Sculier et al., (1986) Cancer Immunol. and Immunother. 23, A65; Sikora et al., (1984) Cancer Letters 23:289-295; Sikora et al., (1988) Analytical Biochem. 172:344-355; all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., (1994) J. Med. Chem. 37:2627-2629; MaGuire, J. (1994) Med. Chem. 37:2129-2131; Burke et al., (1993) J. Med. Chem. 36:425-432 ; and Burke et al. (1992) BioOrganic Med. Chem. Letters 2:1771-1774, all of which are incorporated by reference in their entirety, including any drawings.

Tyrphostins are described in Allen et al., (1993) Clin. Exp. Immunol. 91:141-156; Anafi et al., (1993) Blood 82:12,3524-3529; Baker et al., (1992) J. Cell Sci. 102:543-555; Bilder et al., (1991) Amer. Physiol. Soc. pp. 6363-6143:C721-C730; Brunton et al., (1992) Proceedings of Amer. Assoc. Cancer Rsch. 33:558; Bryckaert et al., (1992) Exp. Cell Research 199:255-261; Dong et al., (1993) J. Leukocyte Biology 53:53-60; Dong et al., (1993) J. Immunol. 151(5):2717-2724; Gazit et al., (1989) J. Med. Chem. 32, 2344-2352; Gazit et al., (1993) J. Med. Chem. 36:3556-3564; Kaur et al., (1994) Anti-Cancer Drugs 5:213-222; King et al., (1991) Biochem. J. 275:413-418; Kuo et al., (1993) Cancer Letters 74:197-202; Levitzki, A., (1992) The FASEB J. 6:3275-3282; Lyall et al., (1989) J. Biol. Chem. 264:14503-14509; Peterson et al., (1993) The Prostate 22:335-345; Pillemer et al., (1992) Int J. Cancer 50:80-85; Posner et al., (1993) Molecular Pharmacology 45:673-683; Rendu et al., (1992) Biol. Pharmacology 44(5):881-888; Sauro and Thomas, (1993) Life Sciences 53:371-376; Sauro and Thomas, (1993) J. Pharm. and Experimental Therapeutics 267(3):119-1125; Wolbring et al., (1994) J. Biol. Chem. 269(36):22470-22472; and Yoneda et al., (1991) Cancer Research 51:4430-4435; all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

### Recombinant DNA Technology

DNA Constructs Comprising a Phosphatase Nucleic Acid Molecule and Cells Containing These Constructs

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and hereby is capable of expressing a polypeptide. The polypeptide may be purified from cells which have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either a eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequence which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a phosphatase of the invention may be obtained by the above-described methods. This region may be retained for its transcriptional termination regulatory sequences, such as termination and polyadenylation. Thus, by retaining the 3′-region naturally contiguous to the DNA sequence encoding a phosphatase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a phosphatase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a phosphatase of the invention, or (3) interfere with the ability of the gene sequence of a phosphatase of the invention to be transcribed by the promoter region sequence. Thus, a promoter region would be operably linked to a DNA sequence if the promoter were capable of effecting transcription of that DNA sequence. Thus, to express a gene encoding a phosphatase of the invention, transcriptional and translational signals recognized by an appropriate host are necessary.

The present invention encompasses the expression of a gene encoding a phosphatase of the invention (or a functional derivative thereof) in either prokaryotic or eukaryotic cells. Prokaryotic hosts are, generally, very efficient and convenient for the production of recombinant proteins and are, therefore, one type of preferred expression system for phosphatases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC119 and the like; suitable phage or bacteriophage vectors may include λgt10, λgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a phosphatase of the invention (or a functional derivative thereof) in a prokaryotic cell, it is necessary to operably link the sequence encoding the phosphatase of the invention to a functional prokaryotic promoter. Such promoters may be either constitutive or, more preferably, regulatable (i.e., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, recA, λacZ λacI, and gal promoters of E. coli, the α-amylase (Ulmanen et al., J. Bacteriol. 162:176-182, 1985) and the ç-28-specific promoters of B. subtilis (Gilman et al., Gene Sequence 32:11-20, 1984), the promoters of the bacteriophages of Bacillus (Gryczan, In: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the phosphatase polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus 35S and 19S, and nopaline synthase promoter and polyadanylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of phosphatases of the invention in insect cells (Jasny, Science 238:1653, 1987; Miller et al., In: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of phosphatases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperate, expression can be repressed or initiated, or are subject to chemical (such as metabolite) regulation.

Expression of phosphatases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al, Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon which encodes the first methionine. For this reason, it is preferable to ensure that the linkage between a eukaryotic promoter and a DNA sequence which encodes a phosphatase of the invention (or a functional derivative thereof) does not contain any intervening codons which are capable of encoding a methionine (i.e., AUG). The presence of such codons results either in the formation of a fusion protein (if the AUG codon is in the same reading frame as the phosphatase of the invention coding sequence) or a frame-shift mutation (if the AUG codon is not in the same reading frame as the phosphatase of the invention coding sequence).

A nucleic acid molecule encoding a phosphatase of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The maker may provide for prototrophy to an auxotrophic host, biocide resistance, e.g., antibiotics, or heavy metals, such as copper, or the like. The selectable maker gene sequence can either be directly lid to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-289, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector, the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, Co1E1, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:4177-4183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bactriol. 33:729-742, 1978).

Preferred eukaryotic plasmids include, for example, BPV, vaccinia, SV40, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, NY, p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the constuct(s) has been prepared for expression, the DNA constuct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a phosphatase of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

Transgenic Animals:

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82:4438-4442, 1985). Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), Harlan Sprague Dawley (Indipolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sanford et al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Terotocarcinomas and Embryonic Stem Cells, A Practical Approach, E. J. Robertson, ed., IRL Press, 1987).

In cases involving random gene integration, a clone containing the secquence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombina-tion (Capecchi, Science 244:1288-1292, 1989). Methods for positive selection of the recombinaton event (i.e., neo resistance) and dual positive-negative selection (i.e., neo resistance and gancyclovir resistance) and the subsequent identification of the desired clones by PCR have been described by Capecchi, supra and Joyner et al. (Nature 338:153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other a animals have been discussed by others (Houdebine and Chourrout, supra; Pursel et al., Science 244:1281-1288, 1989; and Simms et al., Bio/Technology 6:179-183, 1988).

Thus, the invention provides transgenic, nonhuman mammals containing a transgene encoding a kinase of the invention or a gene affecting the expression of the kinase. Such transgenic nonhuman mammals are particularly useful as an in vivo—test system for studying the effect of introduction of a kinase, or regulating the expression of a kinase (i.e., through the introduction of additional genes, antisense nucleic acids, or ribozymes).

A “transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode human kinases. Native expression in an animal may be reduced by providing an amount of antisense RNA or DNA effective to reduce expression of the receptor.

Gene Therapy

Phosphatases or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing a phosphatase coding sequence is inserted into cells, the cells are grown in vitro and then infused in large numbers into patients. In another preferred embodiment, a DNA segment containing a promoter of choice (for example a strong promoter) is transferred into cells containing an endogenous gene encoding phosphatases of the invention in such a manner that the promoter segment enhances expression of the endogenous phosphatase gene (for example, the promoter segment is transferred to the cell such that it becomes directly linked to the endogenous phosphatase gene).

The gene therapy may involve the use of an adenovirus containing phosphatase cDNA targeted to a tumor, systemic phosphatase increase by implantation of engineered cells, injection with phosphatase-encoding virus, or injection of naked phosphatase DNA into appropriate tissue.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction, may be used to inhibit an abnormal, deleterious signal transduction event.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encoding recombinant phosphatase of the invention protein into the targeted cell population (e.g., tumor cells). Methods which are well known to those skilled in the art can be used to construct recombinant viral vectors containing coding sequences (Maniatis et al., Molecular Cloning: A Laboratory Manual, Cold Spring Harbor Laboratory, N.Y., 1989; Ausubel et al., Current Protocols in Molecular Biology, Greene Publishing Associates and Wiley Interscience, N.Y., 1989). Alternatively, recombinant nucleic acid molecules encoding protein sequences can be used as naked DNA or in a reconstituted system e.g., liposomes or other lipid systems for delivery to target cells (e.g., Felgner et al., Nature 337:387-8, 1989). Several other methods for the direct transfer of plasmid DNA into cells exist for use in human gene therapy and involve targeting the DNA to receptors on cells by complexing the plasmid DNA to proteins (Miller, supra).

In its simplest form, gene transfer can be performed by simply injecting minute amounts of DNA into the nucleus of a cell, through a process of microinjection (Capecchi Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with calcium phosphate and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophllic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a phosphatase polypeptide is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid sequence which is capable of being expressed in vivo in an animal and thereby providing or augmenting the function of an endogenous gene which is missing or defective in the animal.

Pharmaceutical Formulations and Routes of Administration

The compounds described herein can be administered to a human patient per se, or in pharmaceutical compositions where it is mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., latest edition.

Routes of Administration:

Suitable routes of administration may, for example, include oral, rectal transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Composition/Formulation:

The pharmaceutical compositions of the present invention may be manufactured in a manner that is itself known, e.g., by means of conventional mixing dissolving, granulating, dragee-making levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Suitable carriers include excipients such as, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium caboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oil, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichloroterafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds may be formulated for parenteral administration by injection, e.g, by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as sending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol a nonpolar surfactant, a water-miscible organic polymer, and an aqueous phase. The cosolvent system may be the VPD co-solvent system. VPD is a solution of 3% w/v benzyl alcohol 8% w/v of the nonpolar sure polysorbate 80, and 65% w/v polyethylene glycol 300, made up to volume in absolute ethinol. The VPD co-solvent syster (VPD:D5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the tyrosine or serine/threonine phosphatase modulating compounds of the invention may be provided as salts with pharmaceutically compatible counterions. Pharmaceutically compatible salts may be formed with many acids, including but not limited to hydrochloric, sulfuric, acetic, lactic, tataric, malic, succinic, etc. Salts tend to be more soluble in aqueous or other protonic solvents that are the corresponding free base forms.

Suitable Dosage Regimens:

Pharmaceutical compositions suitable for use in the preset invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

For any compound used in the methods of the invention, the therapeutically effective dose can be estimated initially from cell culture assays. For example, a dose can be formulated in animal models to achieve a cling concentration range that includes the IC₅₀ as determined in cell culture (i.e., the concentration of the test compound which achieves a half-maximal inhibition of the tyrosine or serine/threonine phosphatase activity). Such information can be used to more accurately determine useful doses in humans.

Toxicity and therapeutic efficacy of the compounds described herein can be determined by standard pharmaceutical procedures in cell cultures or experimental animals, e.g., for determining the LD₅₀ (the dose lethal to 50% of the population) and the Ed₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Dosage amount and interval may be adjusted individually to provide plasma levels of the active moiety which are sufficient to maintain the phosphatase modulating effects, or minimal effective concentration (MEC). The MEC will vary for each compound but can be estimated from in vitro data; e.g., the concentration necessary to achieve 50-90% inhibition of the phosphatase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%.

In cases of local adminison or selective uptake, the effective local concentration of the drug may not be related to plasma concentration.

The amount of composition administered will, of course, be dependent on the subject big treated, on the subject's weight, the severity of the affliction, the manner of administration and the judgment of the prescribing physician.

Packaging:

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the polynucleotide for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition. Suitable conditions indicated on the label may include treatment of a tumor, inhibition of angiogenesis, treatment of fibrosis, diabetes, and the like.

### Functional Derivatives

Also provided herein are functional derivatives of a polypeptide or nucleic acid of the invention. By “functional derivative” is meant a “chemical derivative,” “fragment,” or “variant” of the polypeptide or nucleic acid of the invention, which terms are defined below. A functional derivative retains at least a portion of the function of the protein, for example reactivity with an antibody specific for the protein, enzymatic activity or binding activity mediated through noncatalytic domains, which permits its utility in accordance with the pry invention. It is well known in the art that due to the degeneracy of the genetic code numerous different nucleic acid sequence can code for the same amino acid sequence. Equally, it is also well known in the art that conservative changes in amino acid can be made to arrive at a protein or polypeptide that retains the functionality of the original. In both cases, all permutations are intended to be covered by this disclosure.

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the genes of the invention could be synthesized to give a nucleic acid sequence significantly different from one selected from the group consisting of those set forth in SEQ ID NO:1, SEQ ED NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid formula selected from the group consisting of those set forth in SEQ D NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12, or a derivative thereof Any nucleotide or polynucleotide may be used in this regard, provided that its addition, deletion or substitution does not alter the amino acid sequence of selected from the group consisting of those set forth in SEQ ID NO: 13, SEQ ID NO: 14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24, which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto. All variations of the nucleotide sequence of the phosphatase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

A “chemical derivative” of the complex contains additional chemical moieties not normally a part of the protein. Covalent modifications of the protein or peptides are included within the scope of this invention. Such modifications may be introduced into the molecule by reacting targeted amino acid residues of the peptide with an organic derivatizing agent that is capable of reacting with selected side chains or terminal residues, as described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect or reversing the charge of the lysinyl residues. Other suitable reagents for derivatizing primary amine containing residues include imidoesters such as methyl picolinimidate; pyridoxal phosphate; pyridoxal; chloroborohydride; trinitrobenzenesulfonic acid; O-methylisourea; 2,4 pentanedione; and transaninase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKₐ of the guanidine functional group. Furthermore, these reagents may react with the groups of lysine as well as the arginine alpha-amino group.

Tyrosyl reside are well-known targets of modification for introduction of spectral labels by reaction with aromatic diazonium compounds or tetranitromethane. Most commonly, N-acetylimidizol and tetranitromethane are used to form O-acetyl tyrosyl species and 3-nitro derivatives, respectively.

Carboxyl side groups (aspartyl or glutamyl) are selectively modified by reaction with carbodiimide (R′—N—C—N—R′) such as 1-cyclohexyl-3-(2-morpholinyl(4-ethyl)carbodiimide or 1-ethyl-3-(4-azonia-4,4-dimethylpentyl)carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Derivatization with bifunctional agents is useful, for example, for cross-linking the component peptides of the protein to each other or to other proteins in a complex to a water-insoluble support matrix or to other macromolecular carriers. Commonly used cross-linking agents include, for example, 1,1-bis(diaoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[p-azidophenyl)dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W.H. Freeman & Co., San Francisco, pp. 79-86 (1983)), acetylation of the N-terminal amine, and, in some instances, amidation of the C-terminal carboxyl groups.

Such derivatized moieties may improve the stability, solubility, absorption, biological half-life, and the like. The moieties may alternatively eliminate or attenuate any undesirable side effect of the protein complex and the like. Moieties capable of mediating such effects are disclosed, for example, in Remington's Pharmaceutical Sciences, 18th ed., Mack Publishing Co., Easton, Pa. (1990).

The term “fragment” is used to indicate a polypeptide derived from the amino acid sequence of the proteins, of the complexes having a length less than the full-length polypeptide from which it has been derived. Such a fragment may, for example, be produced by proteolytic cleavage of the full-length protein. Preferably, the fragment is obtained recombinantly by appropriately modifying the DNA sequence encoding the proteins to delete one or more amino acids at one or more sites of the C-terminus, N-terminus, and/or within the native sequence. Fragments of a protein are useful for screening for substances that act to modulate signal transduction, as described herein. It is understood that such fragments may retain one or more characterizing portions of the native complex. Examples of such retained characteristics include: catalytic activity, substrate specificity; interaction with other molecules in the intact cell; regulatory functions; or binding with an antibody specific for the native complex, or an epitope thereof.

Another functional derivative intended to be within the scope of the present invention is a “variant” polypeptide which either lacks one or more amino acids or contains additional or substituted amino acids relative to the native polypeptide. The variant may be derived from a naturally occurring complex component by appropriately modifying the protein DNA coding sequence to add, remove, and/or to modify codons for one or more amino acids at one or more sites of the C-terminus, N-terminus, and/or within the native sequence. It is understood that such variants having added, substituted and/or additional amino acids retain one or more characterizing portions of the native protein, as described above.

A functional derivative of a protein with deleted, inserted and/or substituted amino acid residues may be prepared using standard techniques well-known to those of ordinary skill in the art. For example, the modified components of the functional derivatives may be produced using site-directed mutagenesis techniques (as exemplified by Adelman et al., 1983, DNA 2:183) wherein nucleotides in the DNA coding the sequence are modified such that a modified coding sequence is modified, and thereafter expressing this recombinant DNA in a prokaryotic or eukaryotic host cell, using techniques such as those described above. Alternatively, proteins with amino acid deletions, insertions and/or substitutions may be conveniently prepared by direct chemical synthesis, using methods well-known in the art. The functional derivatives of the proteins typically exhibit the same qualitative biological activity as the native proteins.

The invention also provides methods for determining whether a nucleic acid sequence encodes a phosphatase, according to the invention, which contains one or more characterizing portions of the native complex. As noted, examples of such retained characteristics include: catalytic activity; substrate specificity interaction with other molecules in the intact cell; regulatory functions; or binding with an antibody specific for the native complex, or an epitope thereof. Accordingly, the invention provides an assay analyzing one or more characteristics—in particular, the presence of a catalytic domain—of a polypeptide phosphatase encoded by a given nucleic acid molecule.

To this end, a suitable assay can begin by purifying and quantitating a photphase protein. The protein then can be assayed, for example, by serial dilution and incubation in a buffer (e.g. ABT buffer) comprising a substrate capable of undergoing hydrolysis and optionally a reducing agent capable of increasing any catalytic activity of the polypeptide. Preferably, the substrate is p-nitrophenyl phosphate (pNPP) and the reducing agent is dithiothreitol (DTT), at mM concentrations of 4× and 1× respectively. Incubation can be at room temperature from about 2 minutes to overnight, depending on activity. To stop the reaction, add NaOH, which can be about 100 ul of 10 N NaOH. The suspension can be centrifuged and the supernatant analyzed at an OD of 410 nM to determine whether to protein phosphate exhibited catalytic properties.

### Tables and Description Thereof

Table 1 documents the name of each gene, the classification of each gene product, the positions of the open reading frames within the sequence, and the length of the corresponding peptide. From left to right the data presented is as follows: “Gene Name”, “ID#na”, “ID#aa”, “FL/Cat”, “Superfamily”, “Group”, “Family”, “NA_length”, “ORF Start”, “ORF End”, “ORF Length”, and “AA_length”. “Gene name” refers to name given the sequence encoding the phosphatase or phosphatase-like enzyme. Each gene is represented by “SGP” design followed by an arbitrary number. The SGP name usually represents multiple overlapping sequences built into a single contiguous sequence (a “contig”) The “ID#na” and “ID#aa” refer to the identification numbers given each nucleic acid and amino acid sequence in this patent. “FL/Cat” refers to the length of the gene, with FL indicating full length, and “Cat” indicating that only the catalytic domain is present “Partial” in this column indicates that the sequence encodes a partial protein phosphatase catalytic domain. “Superfamily” identifies whether the gene is a dual specificity phosphatase, a protein tyrosine phosphatase or a serine threonine phosphatase. “Group” and “Family” refer to the phosphatase classification defined by sequence homology and based on previously established phylogenetic (The Protein Phosphatase Factsbook, Nick Tonks, Shirish Shenolikar, Harry Charbonneau, Academic Pr, 2000). “NA_length” refers to the length in nucleotides of the corresponding nucleic acid sequence. “ORF start” refers to the beginning nucleotide of the open reading frame. “ORF end” refers to the last nucleotide of the open reading frame, including the stop codon. “ORF length” refers to the length in nucleotides of the open reading frame. “AA length” refers to the length in amino acids of the peptide encoded in the corresponding nucleic acid sequence.

TABLE 1
Open Reading Frames
424454_2

| Gene Name | ID#na | ID#aa | FL/Cat | Superfamily | Group | Family | NA_length | ORF Start | ORF End | ORF Length | AA_length |
|---|---|---|---|---|---|---|---|---|---|---|---|
| SGP006 | 1 | 13 | FL | Dual Phosphatase | DSP | MKP | 6374 | 34 | 3183 | 3150 | 1049 |
| SGP002 | 2 | 14 | FL | Dual Phosphatase | DSP | MKP | 2732 | 538 | 2535 | 1998 | 665 |
| SGP001 | 3 | 15 | FL | Dual Phosphatase | DSP | MKP | 2260 | 709 | 2205 | 1497 | 496 |
| SGP018 | 4 | 16 | FL | Dual Phosphatase | DSP | MKP | 4361 | 206 | 3609 | 3402 | 1133 |
| SGP003 | 5 | 17 | FL | Dual Phosphatase | DSP | MKP | 1262 | 240 | 902 | 663 | 220 |
| SGP014 | 6 | 18 | FL | Dual Phosphatase | DSP | MKP | 1917 | 31 | 1680 | 1650 | 549 |
| SGP060 | 7 | 19 | FL | Dual Phosphatase | DSP | MKP | 636 | 1 | 636 | 636 | 211 |
| SGP008 | 8 | 20 | FL | Dual Phosphatase | DSP | STYX | 1326 | 1 | 990 | 990 | 329 |
| SGP039 | 9 | 21 | FL | Serine Phosphatase | STP | PP2C | 1083 | 1 | 1083 | 1083 | 360 |
| SGP040 | 10 | 22 | FL | Serine Phosphatase | STP | PP2C | 1725 | 1 | 1725 | 1725 | 574 |
| SGP012 | 11 | 23 | Cat | Tyrosine | RPTP | PTPd | 4719 | 1 | 4719 | 4719 | 1573 |
| | | | | Phosphatase | | | | | | | |
| SGP024 | 12 | 24 | Partial | Tyrosine | RPTP | PTPd | 354 | 1 | 357 | 357 | 118 |
| | | | | Phosphatase | | | | | | | |

Table 2 lists the following features of the genes described in this application: chromosomal localization, single nucleotide polymorphisms (SNPs), representation in dbEST, and repeat regions. From left to right the data presented is as follows: “Gene Name”, “ID#na”, “ID#aa”, “FL/Cat”, “Superfamily”, “Group”, “Family”, “Chromosome”, “SNPs”, “dbEST_hits”, & “Repeats”. The contents of the first 7 columns (i.e., “Gene Name”, “ID#na”, “ID#aa”, “FL/Cat ”, “Superfamily”, “Group”, “Family”) are as described above for Table 1. “Chromosome” refers to the cytogenetic localization of the gene. Information in the “SNPs” column describes the nucleic acid position and degenerate nature of candidate single nucleotide polymorphisms (SNPs. “dbEST hits” lists accession numbers of entries in the public database of ESTs (dbEST, http://www.ncbi.nlm.nih.gov/dbEST/index.html) that contain at least 100 bp of 100% identity to the corresponding gene. These ESTs were identified by blastn of dbEST. “Repeats” contains information about the location of short sequences, approximately 20 bp in length, that are of low complexity and that are present in several distinct genes. These repeats were identified by blastn of the DNA sequence against the non-redundant nucleic acid database at NCBI (nrna). To be included in this repeat column, the sequence typically has 100% identity over its lengh and is presnt in at least 5 different genes.

TABLE 2
CHR, SNPs, dbEST, Repeats
424454_2

| Gene Name | ID#na | ID #aa | FL/Cat | Superfamily | Group | Family | Chromosome | 8NPa | dbE9T_hits | Repeats |
|---|---|---|---|---|---|---|---|---|---|---|
| SGP008 | 1 | 13 | FL | Dual Phosphatase | DSP | MKP | 12p21.3-g22 | [symbol] | BE783082.1, AI851213.1, | AI 8750-8010, |
| | | | | | | | | | BE250079.1 | 5750-5770 |
| SGP002 | 2 | 14 | FL | Dual Phosphatase | DSP | MKP | 12p11.1-p121 | none | BE807795; | 2810-2831 |
| SGP001 | 3 | 15 | FL | Dual Phosphatase | DSP | MKP | Xg11.1-113 | none | AI272211, BF205586, | 679-598 |
| SGP018 | 4 | 16 | FL | Dual Phosphatase | DSP | MKP | NA | [symbol] | BF114881 | 883-1014 |
| SGP003 | 5 | 17 | FL | Dual Phosphatase | DSP | MKP | CHR10 | none | none | 311-334 |
| SGP014 | 6 | 18 | FL | Dual Phosphatase | DSP | MKP | [symbol] | none | AA723271, AW444890.1, | none |
| | | | | | | | | | AW435513.1 | |
| SGP080 | 7 | 19 | FL | Dual Phosphatase | DSP | MKP | [symbol] | none | BP207232, BF314818, | none |
| | | | | | | | | | AW9532101 | |
| SGP006 | 8 | 20 | FL | Dual Phosphatase | DSP | STYX | 20q112 | [symbol] | AW406820.1, BF377364.1, | 1251-1270 |
| | | | | | | | | | AW691298.1 | |
| SGP039 | 9 | 21 | FL | Serine | STP | PP2C | NA | none | BE147139.1 | none |
| | | | | Phosphatase | | | | | | |
| SGP040 | 10 | 22 | FL | Serine | STP | PP2C | $$21.3 | none | AV708533.1, AV705571.1, | none |
| | | | | Phosphatase | | | | | AV710601.1 | |
| SGP012 | 11 | 23 | Cat | Tyrosine | RPTP | PTPd | NA | none | AL042532.1, AD81571, | 3105-3124 |
| | | | | Phosphatase | | | | | AW872877 | |
| SGP024 | 12 | 24 | Partial | Tyrosine | RPTP | PTPd | NA | none | none | none |
| | | | | Phosphatase | | | | | | |

Table 3 Lists the extent and the boundaries of the phosphatase catalytic domains. The column headings are: “Gene Name”, “ID#na”, “ID#aa”, “FL/Cat”, “Domain”, “Phos_start”, “Phos_end”, “Profile_start”, “Profile_end”. The contents columns “Gene Name”, “ID#na”, “ID#aa”, “FL/Cat”, are as described above for Table 1. “Phos Start”, “Phos End”, “Profile Start” and “Profile End” refer to data obtained using a Hidden-Markov Model to define catalytic range boundaries (http://pfam.wustl.edu/index.html). The boundaries of the catalytic domains within the overall protein are noted in the “Phos Start” and “Phos End” columns. Three profiles were used, one for dual specificity phosphatases (DSP) which is 173 amino acids long;, one for STPs, which is 301 amino acids long; and one for PTPs, which is 264 amino acids long. (The profiles used are described in http://pfam.wustl.edu/). Proteins in which the profile recognizes a full length catalytic domain have a “Profile Start” of 1 and, for the three families, the following Profile Ends: 173 for DSP, 301 for STPs, and 264 for PTPs. Genes which have a partial catalytic domain will have a “Profile Start” of greater than 1 (indicating that the beginning of the phosphatase domain is missing, and/or a “Profile End” of less than 261 (indicating that the C-terminal end of the phosphatase domain is missing. Each of the sequences encompasses a complete catalytic domain, except for SGP024, which has a partial catalytic domain represents amino acids 205 to 264 of the PTP profile.

TABLE 3
Phosphatase Domains
424454_2

| Gene Name | ID#na | ID#aa | FL/Cat | Domain | Phos_start | Phos_end | Profile_start | Profile_end |
|---|---|---|---|---|---|---|---|---|
| SGP006 | 1 | 13 | FL | DSP | 308 | 446 | 1 | 173 |
| SGP002 | 2 | 14 | FL | DSP | 158 | 297 | 1 | 173 |
| SGP001 | 3 | 15 | FL | DSP | 307 | 441 | 1 | 173 |
| SGP018 | 4 | 16 | FL | DSP | 185 | 330 | 1 | 173 |
| SGP003 | 5 | 17 | FL | DSP | 54 | 199 | 1 | 173 |
| SGP014 | 6 | 18 | FL | 2 DSPs (37-181 & 368-520) | 37 & 368 | 181 & 520 | 1 | 173 |
| SGP060 | 7 | 19 | FL | DSP | 61 | 204 | 1 | 173 |
| SGP008 | 8 | 20 | FL | DSP | 98 | 235 | 1 | 173 |
| SGP039 | 9 | 21 | FL | Protein phosphatase 2C | 91 | 344 | 1 | 301 |
| SGP040 | 10 | 22 | FL | Protein phosphatase 2C | 209 | 497 | 1 | 301 |
| SGP012 | 11 | 23 | Cat | PTP | 1010 | 1259 | 1 | 264 |
| SGP024 | 12 | 24 | Partial | PTP | 3 | 58 | 205 | 264 |

Table 4 describes the results of Smith Waterman similarity searches (Matrix: Pam100; gap open/extension penalties 12/2) of the amino acid sequences it the NCBI database of non-redundant protein sequences (http://www.ncbi.nlm.nih.gov/Entrez/protein.html). The column headings are: “Gene Name”, “ID#na”, “ID#aa”, “FL/Cat”, “Family”, “Pscore”, “aa_length”, “aa_ID_match”, “%Identity”, “%Similar”, “ACC#_nraa_match”, “Description”, “Query start”, “Query end”, “Target start”, and “Target end”. The contents of columns, “Gene Name”, “ID#na”, “Id#aa”, “FL/Cat”, and “Family” are as described above for Table 1. “Pscore” refers to the Smith Waterman probability score. This number approximates the probability that the alignment occurred by chance. Thus, a very low number, such as 2.10E-64, indicates that there is a very significant match between the query and the database target. “aa_length” refers to the length of the protein in amino acids. “aa_ID_match” indicates the number of amino acids that were identical in the alignment “% Identity” list the percent of nucleotides that were identical over the aligned region. “% Similarity” lists the percent of amino acids that were similar over the alignment “ACC#nraa_match” list the accession number of the most similar protein in the NCBI database of non-redundant proteins. “Description” contains the name of the most similar protein in the NCBI database of non-redundant proteins. “Query start” refers to the amino acid number in the phosphatase (“Query”) at which the alignment begins. “Query end” refers to the amino acid number the phosphatase (“Query”) at which the alignment ends. “Target start” refers to the amino acid number in the Smith Waterman hit (“Target”) from NRAA at which the alignment begins. “Target end” refers to the amino acid number in the Smith Waterman hit (“Target”) from NRAA at which the alignment ends. Note that for SGP006 there three entries, and for SGP014 there are two entries. These additional rows describe different regions of alignments with different database “Targets” (see below for detailed descriptions).

TABLE 4
Smith Waterman

| Gene | | | | | | | | | | |
|---|---|---|---|---|---|---|---|---|---|---|
| Name | ID#na | ID#aa | FL/Cat | Family | Pscore | aa_length | aa_ID_match | % Identity | % Similar | ACC#_prss_match |
| SGP006 | 1 | 13 | FL | MKP | 0 | 1049 | 715 | 100 | 100 | BAA92536.1 |
| SGP006 | 1 | 13 | FL | MKP | 1.50E−99 | 1049 | 248 | 46 | 59 | BAA89534.1 |
| SGP006 | 1 | 13 | FL | MKP | 6.80E−58 | 1049 | 119 | 41 | 59 | NP_060327.1 |
| SGP002 | 2 | 14 | FL | MKP | 1.10E−157 | 565 | 304 | 46 | 60 | NP_004411.1 |
| SGP001 | 3 | 15 | FL | MKP | 8.30E−133 | 498 | 250 | 47 | 60 | BAA89534.1 |
| SGP018 | 4 | 16 | FL | MKP | 2.20E−27 | 1133 | 79 | 45 | 63 | NP_057448.1 |
| SGP003 | 5 | 17 | FL | MKP | 3.40E−54 | 220 | 91 | 49 | 68 | NP_057448.1 |
| SGP014 | 6 | 18 | FL | MKP | 7.50E−122 | 549 | 198 | 88 | 88 | NP_057448.1 |
| SGP014 | 6 | 18 | FL | MKP | 8.20E−36 | 549 | 75 | 45 | 65 | NP_004081.1 |
| SGP060 | 7 | 19 | FL | MKP | 1.10E−48 | 211 | 86 | 53 | 72 | NP_057448.1 |
| SGP008 | 8 | 20 | FL | STYX | 4.40E−172 | 329 | 260 | 92 | 92 | CAC10008.1 |
| SGP039 | 9 | 21 | FL | PP2C | 1.00E−106 | 360 | 154 | 96 | 99 | AAD17235.1 |
| SGP040 | 10 | 22 | FL | PP2C | 0 | 574 | 574 | 100 | 100 | NP_060914.1 |
| SGP012 | 11 | 23 | Cat | PTPd | 0.00E+00 | 1573 | 1053 | 60 | 70 | NP_031981.1 |
| SGP024 | 12 | 24 | Partial | PTPd | 5.90E−54 | 118 | 90 | 76 | 82 | CAA38068.1 |

| Gene | | | | | |
|---|---|---|---|---|---|
| Name | Description | Query start | Query end | Target start | Target end |
| SGP006 | KIAA1298 protein [Homo sapeins] | 322 | 1049 | 11 | 738 |
| SGP006 | MAP kinase phosphatase [Dropsophila melanogaster] | 120 | 477 | 199 | 551 |
| SGP006 | Hypothetical protein FLJ20515 [Homo sapeins] | 1 | 263 | 1 | 283 |
| SGP002 | dual specificity phosphatase 8 [Homo sapeins] | 13 | 565 | 14 | 625 |
| SGP001 | MKP [Dropsophila melanogaster] | 1 | 442 | 1 | 522 |
| SGP018 | Protein phosphatase LOC51207 [Homo sapeins] | 162 | 334 | 23 | 194 |
| SGP003 | Protein phosphatase LOC51207 [Homo sapeins] | 22 | 206 | 12 | 197 |
| SGP014 | Protein phosphatase LOC51207 [Homo sapeins] | 324 | 549 | 1 | 198 |
| SGP014 | Dual specificity phosphatase 3 [Homo sapeins] | 20 | 179 | 6 | 174 |
| SGP060 | Protein phosphatase LOC51207 [Homo sapeins] | 48 | 205 | 31 | 191 |
| SGP008 | Novel protein [Homo sapeins] | 63 | 329 | 1 | 275 |
| SGP039 | PP 2C [Mus musculus] | 133 | 299 | 1 | 167 |
| SGP040 | Pyruvate dehydrogenase phosphatase [Homo sapeins] | 1 | 574 | 1 | 574 |
| SGP012 | Embryonic slam cell phosphatase [Mus musculus] | 1 | 1559 | 1 | 1704 |
| SGP024 | Protein tyrosine phosphatase delta [Homo sapiens] | 1 | 118 | 1165 | 1262 |

Table 5 shows the results of a gene expression analysis of selected phosphatases presented in this application using a microarray of cDNAs derived from 499 tissues and cell lines. The cDNAs were spotted on nylon and probed with labeled phosphatase genes, as described in Materials and Methods below. The phosphatase probes were PCR cloned from genomic exons. Data presentation from left to right is as follows: “ID”: number of the sample; “Sample name”; “T/N”, tumor or normal tissue; “Type”, tissue of origin; “Tissue/cell line”, sample is derived from tissue or from a cultured cell line; “notes”: additional information about the sample; “Treatment”: chemical or physical treatment of the tissue or cell line; “p53” refers to the status, mutant or wild-type, of the p53 gene in the source samples. Normalized expression values are presented for each gene referred to by its SGP and SEQ_ID# on the subsequent columns. Genes represented in Table 5 are: SGP003, SGP060, and SGP018.

Images of the blots containing the probed tissue arrays are included.

TABLE 5
Tissue Array

| | | | | | | | | | ID#NA_5, | ID#NA_7, |
|---|---|---|---|---|---|---|---|---|---|---|
| ID | Sample_name | T/N | Type | Tissue/cell line | Notes | Treatment | p53 | ID#NA_4, SGP018 | SGP003 | SGP060 |
| 7 | cerebellum - h | N | neuro | tissue | | none | | 736 | 0 | 1,924 |
| 195 | 458 medullo mRNA | N | neuro | tissue | | none | | 717 | 2,956 | 0 |
| 11 | fetal kidney - h | N | renal | tissue | | none | | 708 | 3,029 | 341 |
| 41 | Duodenum - h | N | col | tissue | | none | | 633 | 0 | 0 |
| 8 | pituitary gland - h | N | neuro | tissue | | none | | 627 | 2,928 | 3,003 |
| 14 | salivary gl. - h | N | salivary | tissue | | none | | 608 | 2,044 | 193 |
| 31 | trachea - h | N | trachea | tissue | | none | | 580 | 0 | 0 |
| 44 | testis - h | N | testes | tissue | | none | | 535 | 0 | 457 |
| 65 | HT 154 | T | HNS | tissue | | none | | 496 | 1,852 | 109 |
| 185 | ACHN | T | renal | cell line | Renal adenocarcinomea | none | | 495 | 1,114 | 0 |
| 1 | adrenal gland - h | N | adrenal | tissue | | none | | 470 | 0 | 804 |
| 10 | placenta - h | N | plac | tissue | | none | | 459 | 294 | 0 |
| 25 | HPAEC | N | endo | cell line | renal proximal tubule epithelial cells | none | | 458 | 133 | 226 |
| 6 | pancreas - h | N | pan | tissue | | none | | 451 | 1,691 | 0 |
| 377 | OVCAR-5 - 7 | T | OV | cell line | | 400 ng/ml noco-24 | mutant | 448 | 0 | 0 |
| 17 | heart - h | N | heart | tissue | | none | | 447 | 288,691 | 426 |
| 43 | Salivary gl. - h | N | salivary | tissue | | none | | 446 | 0 | 68 |
| 13 | fetal liver - h | N | liver | tissue | | none | | 437 | 1,858 | 0 |
| 46 | h keratinocytes 2/25/92 #10 | T | keratinocyte | cell line | | unknown | | 433 | 434 | 155 |
| 21 | liver - h | N | liver | tissue | | none | | 408 | 0 | 56 |
| 23 | lung - h | N | lung | tissue | | none | | 406 | 1,349 | 0 |
| 457 | SF-268-6 | T | neuro | cell line | | 10 um cisplatin | mutant | 405 | 0 | 0 |
| 38 | fetal liver - h | N | liver | tissue | | none | | 393 | 160 | 41 |
| 350 | HT29 - 4 | T | col | cell line | | 3 mM HU | mutant | 393 | 0 | 0 |
| 26 | TCGP | T | testes | tissue | | none | | 384 | 831 | 0 |
| 324 | Malone-3M | T | renal | cell line | Malignant melanoma, metastasia to | none | | 382 | 1,223 | 279 |
| | | | | | lung | | | | | |
| 114 | SF-539 | T | neuro | cell line | Olioblastoma | none | | 375 | 0 | 268 |
| 229 | HL-60 | T | col | cell line | PML Peripheral blood, | none | | 358 | 0 | 0 |
| | | | | | promyelocytic leukemia | | | | | |
| 30 | RPTEC | N | endo | cell line | mammary epithelial cells | none | | 352 | 5,308 | 0 |
| 375 | OVCAR-5 - 6 | T | OV | cell line | | 10 uM cisplatin | mutant | 350 | 0 | 0 |
| 465 | AngioTest1-13 | N | HUVEC | cell line | 10 mn stimulation with PDGF | PDGF | | 341 | 0 | 0 |
| 93 | HT385 | T | lung | tissue | | none | | 339 | 370 | 151 |
| 111 | A549/ATCC | T | lung | cell line | Lung carcinoma | none | | 336 | 2,559 | 190 |
| 423 | C33A - 8 | T | cervical | cell line | | 400 ng/ml noco-48 | mutant | 333 | 0 | 0 |
| 45 | 458 medullo RNA | T | neuro | | | none | | 333 | 0 | 348 |
| 354 | HT29 - 6 | T | col | cell line | | 10 uM cisplatin | mutant | 328 | 0 | 0 |
| 15 | fetal lung - h | N | lung | tissue | | none | | 323 | 4,701 | 0 |
| 483 | Prostate_sample MG-6 | unknown | pro | unknown | | unknown | | 321 | 0 | 0 |
| 143 | UO-31 | T | renal | cell line | | none | | 319 | 0 | 5 |
| 5 | brain - h | N | neuro | tissue | | none | | 316 | 0 | 1,102 |
| 468 | AngioTest1-3 | T | endo | cell line | HcLa25X DEF-MES for Hypoxia, | 25X DEF-MES | | 304 | 0 | 0 |
| | | | | | 4 h | | | | | |
| 98 | OVCAR-8 | T | OV | cell line | | none | | 302 | 340 | 0 |
| 76 | HT317 | T | lung | tissue | | none | | 300 | 0 | 0 |
| 256 | MDA-N | T | breast | cell line | | none | | 298 | 1,080 | 0 |
| 83 | HELA-9h-031899 | T | endo | cell line | | | | 294 | 0 | 28 |
| 223 | HT382 | T | lung | tissue | | none | | 293 | 0 | 282 |
| 22 | Spleen - h | N | heme | tissue | | none | | 291 | 3,580 | 0 |
| 25 | testis - h | N | testes | tissue | | none | | 290 | 0 | 197 |
| 50 | HT213 | T | kidney | tissue | | none | | 285 | 347 | 0 |
| 123 | RPMI 8226 | T | LEU | cell line | Multiple myeloma | none | | 282 | 0 | 106 |
| 201 | stomach - h | N | col | tissue | | none | | 282 | 0 | 0 |
| 490 | Prostate_sampleMG - 10 | unknown | neuro | unknown | NIH3T3 vector | unknown | | 279 | 0 | 0 |
| 251 | HT382-normal | N | lung | tissue | | none | | 279 | 0 | 60 |
| 378 | MCF-7 - 1 | T | breast | cell line | normal/10% FBS | none | wt | 274 | 0 | 0 |
| 489 | OVCAR-5 - 5 | T | OV | cell line | | 2 uM AUR2 inhibitor | mutant | 273 | 0 | 0 |
| 121 | HL-60 | T | col | cell line | PML Peripheral blood, | none | | 273 | 0 | 0 |
| | | | | | promyslocytic leukemia | | | | | |
| 323 | BioMarker_BS-13 | N | endo | cell line | HUVEC VEGF + 5416 - 24 h | VEGF | | 273 | 0 | 0 |
| 265 | KHOS poly A+ | T | bone | cell line | | none | | 271 | 801 | 83 |
| 318 | A498 | T | renal | cell line | Kidney carcinoma | none | | 268 | 221 | 0 |
| 139 | HCT-15 | T | col | cell line | Colon adenocarcinoma | none | | 267 | 0 | 0 |
| 426 | U2OS - 2 | T | bone | cell line | low serum/0.1% FBS | low sereum | mutant | 267 | 0 | 0 |
| 105 | NCI-H322M | T | lung | cell line | Lung Br. A./Lung | none | | 267 | 44 | 58 |
| | | | | | bronchiolosveolar carcinoma | | | | | |
| 85 | HT146 | T | kidney | tissue | | none | | 266 | 1,305 | 0 |
| 411 | C33A - 2 | T | cervical | cell line | low serum/0.1% FBS | low sereum | mutant | 265 | 0 | 0 |
| 464 | AngioTest1-1 | T | endo | cell line | HcLa25X DEF-MES for Hypoxia, | 25X DEF-MES | | 260 | 0 | 0 |
| | | | | | 0 h | | | | | |
| 469 | Prostate_sampleMG - 21 | unknown | pan | unknown | Hu294 | unknown | | 258 | 0 | 0 |
| 254 | MDA-MB-435 | T | breast | cell line | | none | | 254 | 0 | 153 |
| 82 | HT335 | T | pan | tissue | | none | | 253 | 0 | 176 |
| 459 | SF-268-7 | T | neuro | cell line | | 400 ng/ml noco-24 | mutant | 253 | 0 | 0 |
| 106 | SNB-75 | T | neuro | cell line | Astrocytoma | none | | 251 | 655 | 40 |
| 92 | HELA-10h-031899 | T | endo | cell line | | | | 250 | 0 | 0 |
| 37 | Skeletal muscle - h | N | muscle | tissue | | none | | 250 | 0 | 113 |
| 288 | HT394 | T | lung | tissue | | none | | 242 | 0 | 0 |
| 319 | HT393 | T | lung | tissue | | none | | 240 | 0 | 106 |
| 277 | HT338 | T | lung | tissue | | none | | 239 | 82 | 0 |
| 237 | UO-31 | T | renal | cell line | | none | | 238 | 0 | 0 |
| 456 | EKVX - 4 | T | lung | cell line | | 3 mM HU | mutant | 237 | 0 | 0 |
| 382 | MCF-7 - 3 | T | breast | cell line | | 200 uM mimosine | wt | 236 | 0 | 0 |
| 410 | SW480 - 6 | T | col | cell line | | 10 um cisplatin | mutant | 234 | 0 | 0 |
| 476 | AngioTest1-7 | N | endo | cell line | HUVEC unstimulated/control | none | | 234 | 0 | 0 |
| 369 | SF539 - 6 | T | neuro | cell line | | 10 uM cisplatin | wt | 234 | 0 | 0 |
| 379 | ADR-RES - 5 | T | breast | cell line | | 2 uM AUR2 inhibitor | mutant | 232 | 0 | 0 |
| 74 | HT314 | T | MG | tissue | | none | | 225 | 730 | 172 |
| 151 | MCF7 | T | breast | cell line | Breast adenocarcinoma, pleural | none | | 225 | 68 | 0 |
| | | | | | effusion | | | | | |
| 436 | U2OS - 7 | T | bone | cell line | | 400 ng/ml noco-24 | mutant | 224 | 0 | 0 |
| 227 | MOLT-4 | T | LEU | cell line | ALL Peripheral blood, acute | none | | 223 | 0 | 0 |
| | | | | | lymphoblastic leukemia | | | | | |
| 359 | SF539 - 1 | T | neuro | cell line | normal/10% FBS | none | wt | 222 | 0 | 0 |
| 412 | SW480 - 7 | T | col | cell line | | 400 ng/ml noco-24 | mutant | 221 | 0 | 0 |
| 304 | HOP-62 | T | lung | cell line | Lung adenocarcinoma | none | | 219 | 0 | 0 |
| 29 | thyroid gland - h | N | thyroid | tissue | | none | | 217 | 383 | 0 |
| 399 | HeLa - 8 | T | endo | cell line | | 400 ng/ml noco-48 | HPV E6 | 215 | 0 | 0 |
| 146 | NCI-H322M | T | lung | cell line | lung Br. A./Lung | none | | 215 | 0 | 38 |
| | | | | | bronchioloaeveolar carcinoma | | | | | |
| 218 | BioMarker_BS-6 | N | endo | cell line | HUVEC VEGF - 6 h | VEGF | | 213 | 0 | 0 |
| 4 | mammary gland - h | N | breast | tissue | | none | | 213 | 870 | 0 |
| 305 | MDA-MB-231 | T | breast | cell line | Breast adenocarcinoma, pleural | none | | 210 | 403 | 0 |
| | | | | | effusion | | | | | |
| 148 | NCI-H460 | T | lung | cell line | Lung large cell carcinoma | none | | 210 | 0 | 297 |
| 202 | Heart - h | N | heart | tissue | h choriocarcinoma | none | | 208 | 0 | 0 |
| 80 | HT327 | T | lung | tissue | | none | | 208 | 0 | 0 |
| 34 | HCAEC | N | endo | cell line | | none | | 207 | 884 | 14 |
| 104 | SNB-19 | T | neuro | cell line | Glioblastoma | none | | 207 | 0 | 11 |
| 286 | HT392 | T | lung | tissue | | none | | 206 | 412 | 366 |
| 312 | SW-620 | T | col | cell line | Colon adenocarcinoma, lymph node | none | | 204 | 0 | 123 |
| | | | | | metastasis | | | | | |
| 491 | Prostate_sampleMG - 11 | unknown | neuro | unknown | NIH3T3 EWS/FLI1 | unknown | | 202 | 0 | 0 |
| 156 | SF-268 | T | neuro | cell line | Glioblastoma | none | | 202 | 0 | 157 |
| 107 | NCI-H460 | T | lung | cell line | Lung large cell carcinoma | none | | 201 | 0 | 0 |
| 101 | NCI-H23 | T | lung | cell line | Lung adenocarcinoma | none | | 201 | 0 | 0 |
| 441 | WI 38 - 5 | N | lung | cell line | | 2 uM AUR2 inhibitor | wt | 200 | 0 | 0 |
| 380 | MCF-7 - 2 | T | breast | cell line | low serum/0.1% FBS | low sereum | wt | 200 | 0 | 0 |
| 67 | HT169 | T | pro | tissue | | none | | 200 | 0 | 0 |
| 118 | K-562 | T | LEU | cell line | CML Chronic myelogenous | none | | 199 | 0 | 0 |
| | | | | | leukemis | | | | | |
| 403 | H1299 - 6 | T | lung | cell line | | 10 uM cisplatin | mutant | 199 | 0 | 0 |
| 310 | HCC-2998 | T | col | cell line | | none | | 196 | 0 | 0 |
| 433 | WI38 - 1 | N | lung | cell line | normal/10% FBS | none | wt | 196 | 0 | 0 |
| 53 | fetal lung - h | N | lung | tissue | | none | | 196 | 0 | 0 |
| 314 | COLO 205 | T | col | cell line | Colon adenocarcinoma | none | | 194 | 0 | 242 |
| 122 | SNI2C | T | neuro | cell line | | none | | 194 | 0 | 0 |
| 453 | SF-268-4 | T | neuro | cell line | | 3 mM HU | mutant | 193 | 0 | 0 |
| 20 | spinal cord - h | N | neuro | tissue | | none | | 193 | 13,667 | 387 |
| 97 | HOP-92 | T | lung | cell line | Lung large cell carcinoma | none | | 192 | 0 | 365 |
| 77 | Medulloblastoma #425 11/8 | T | neuro | tissue | h Wilms' tumor | none | | 190 | 0 | 138 |
| 259 | adrenal gland - h | N | adrenal | tissue | | none | | 185 | 4,748 | 0 |
| 257 | Y79 poly A+ | T | retinal | cell line | h retinoblastoma | none | | 185 | 0 | 0 |
| 79 | HELA-2h-031899 | T | endo | cell line | | | | 184 | 82 | 191 |
| 155 | Hs 578T | T | breast | cell line | Ductal carcinoma | none | | 183 | 1,362 | 142 |
| 35 | Pancreas - h | N | pan | tissue | h embryonic palstal mesenchyme | none | | 183 | 651 | 0 |
| 55 | heart - h | N | heart | tissue | | none | | 182 | 2 | 0 |
| 187 | UACC-62 | T | mel | cell line | | none | | 180 | 311 | 18 |
| 418 | H1299 - 2 | T | lung | cell line | low serum/0.1% FBS | low sereum | mutant | 179 | 0 | 0 |
| 33 | uterus - h | N | uterus | tissue | | none | | 179 | 0 | 0 |
| 216 | BioMarker_BS-2 | N | endo | cell line | HUVEC control - 1 h | none | | 178 | 822 | 197 |
| 381 | ADR-RES - 6 | T | breast | cell line | | 10 uM cisplatin | mutant | 174 | 0 | 0 |
| 103 | NCI-H226 | T | lung | cell line | Lung aquamous ca | none | | 173 | 0 | 0 |
| 269 | SA-OS (Mundy) poly A+ | T | bone | cell line | h osteogenic sarcoma, primary | none | | 172 | 0 | 24 |
| 63 | HT138 | T | kidney | tissue | | none | | 172 | 0 | 38 |
| 108 | U251 | T | neuro | cell line | Glioblastoma | none | | 171 | 1,326 | 0 |
| 452 | EKVX - 2 | T | lung | cell line | low serum/0.1% FBS | low sereum | mutant | 171 | 0 | 0 |
| 245 | SK-MEL-5 | T | mel | cell line | Malignant melanoma, metastasis to | none | | 171 | 0 | 62 |
| | | | | | axillary node | | | | | |
| 154 | SNB-75 | T | neuro | cell line | Astrocytoma | none | | 170 | 168 | 0 |
| 466 | AngioTest1-2 | T | endo | cell line | HsLa25X DEF-MES for Hypoxin, | 25X DEF-MES | | 169 | 0 | 0 |
| | | | | | 1 h | | | | | |
| 140 | SK-MEL-2 | T | mel | cell line | Malignant melanoma, metastasis to | none | | 168 | 751 | 0 |
| | | | | | skin of thigh | | | | | |
| 442 | Hs68 - 2 | N | lung | cell line | low serum/0.1% FBS | low sereum | wt | 166 | 0 | 0 |
| 371 | SF539 - 7 | T | neuro | cell line | | 400 ng/ml noco-24 | wt | 166 | 0 | 0 |
| 275 | HT334 | T | MG | tissue | | none | | 166 | 174 | 0 |
| 60 | HT170 | T | pro | tissue | | none | | 164 | 0 | 0 |
| 263 | h fibroblasts3/31/92 #12 | T | fibroblast | cell line | metastasis to supraoribital area | unknown | | 164 | 756 | 117 |
| 416 | H1299 - 1 | T | lung | cell line | normal/10% FBS | none | mutant | 163 | 0 | 0 |
| 493 | Prostate_sampleMG - 13 | unknown | neuro | cell line | TC-71 Ewings tumor derived cell | none | | 162 | 0 | 0 |
| | | | | | line | | | | | |
| 144 | SK-MEL-28 | T | mel | cell line | Malignant melanoma | none | | 158 | 0 | 0 |
| 126 | Caki-1 | T | renal | cell line | Clear cell carcinoma, renal primary, | none | | 157 | 296 | 0 |
| | | | | | metastasis to skin | | | | | |
| 326 | Hs 578T | T | breast | cell line | Ductal carcinoma | none | | 155 | 0 | 0 |
| 470 | AngioTest1-4 | T | liver | cell line | HepG2 25X DEF-MES for Hypoxia, | 25X DEF-MES | | 155 | 0 | 0 |
| | | | | | 0h | | | | | |
| 81 | HELA-4h-031899 | T | endo | cell line | | | | 155 | 739 | 0 |
| 492 | Prostate_sampleMG - 12 | unknown | neuro | cell line | TC-32 Ewings tumor derived cell | none | | 152 | 0 | 0 |
| | | | | | line | | | | | |
| 281 | pituitary gland - h | N | neuro | tissue | | none | | 151 | 0 | 218 |
| 404 | SW480 - 3 | T | col | cell line | | 200 um mimosine | mutant | 151 | 0 | 0 |
| 135 | SW-620 | T | col | cell line | Colon adenocarcinoma, lymph node | none | | 151 | 52 | 318 |
| | | | | | metastasis | | | | | |
| 181 | KB poly A+ | T | unknown | cell line | h spidermoid cancer | none | | 148 | 0 | 0 |
| 357 | HT29 - 8 | T | col | cell line | | 400 ng/ml noco-48 | mutant | 148 | 0 | 0 |
| 451 | SF-268-3 | T | neuro | cell line | | 200 uM mimosine | mutant | 147 | 0 | 0 |
| 116 | CCRF-CEM | T | LEU | cell line | ALL Acuts lymphobilastic leukemia | none | | 147 | 1,543 | 202 |
| 70 | HT190 | T | kidney | tissue | | none | | 147 | 676 | 0 |
| 290 | HT312 | T | lung | tissue | | none | | 144 | 0 | 31 |
| 215 | HT371 | T | lung | tissue | | none | | 144 | 75 | 98 |
| 308 | PC-3 | T | pro | cell line | Prostate adenocarcinoma | none | | 144 | 139 | 0 |
| 372 | OVCAR-5 - 4 | T | OV | cell line | | 3 mM HU | mutant | 142 | 0 | 0 |
| 32 | HMEC | N | endo | cell line | coronary artery endothelial cells | none | | 140 | 986 | 0 |
| 197 | fetal brain - h | N | neuro | tissue | | none | | 140 | 0 | 0 |
| 222 | BioMarker_BS-8 | N | endo | cell line | HUVEC 5416-1 h | SU5416 | | 138 | 191 | 0 |
| 296 | HT162 | T | OV | tissue | | none | | 137 | 0 | 0 |
| 2 | lymph node - h | N | heme | tissue | | none | | 136 | 3,663 | 323 |
| 307 | PT cells poly A+ | T | unknown | cell line | | none | | 133 | 0 | 66 |
| 478 | AngioTest1-8 | N | endo | cell line | HUVEC 10 mn stimulation with | VEGF | | 133 | 0 | 0 |
| | | | | | VEGF | | | | | |
| 358 | OVCAR-4 - 4 | T | OV | cell line | | 3 mM HU | wt | 133 | 0 | 0 |
| 19 | kidney - h | N | renal | tissue | | none | | 131 | 2,148 | 55 |
| 145 | UACC-62 | T | mel | cell line | | none | | 130 | 0 | 42 |
| 261 | neruoblastoma RNA | T | neuro | tissue | | none | | 129 | 0 | 395 |
| 388 | MCF-7 - 6 | T | breast | cell line | | 10 uM cisplatin | wt | 128 | 0 | 0 |
| 165 | Caki-1 | T | renal | cell line | Clear cell carcinoma, renal primary, | none | | 128 | 134 | 252 |
| | | | | | metastasis to skin | | | | | |
| 313 | HT 192 | T | MG | tissue | | none | | 127 | 656 | 290 |
| 395 | HeLa - 6 | T | endo | cell line | | 10 uM cisplatin | HPV E6 | 127 | 0 | 0 |
| 24 | stomach - h | N | col | tissue | | none | | 124 | 4,601 | 47 |
| 295 | 458 medullo RNA | T | neuro | | | none | | 124 | 0 | 0 |
| 337 | A549 - 4 | T | lung | cell line | | 3 mM HU | wt | 124 | 0 | 0 |
| 115 | OVCAR - 3 | T | OV | cell line | Ovary adenocarcinoma | none | | 124 | 959 | 0 |
| 225 | BioMarker_BS-11 | N | endo | cell line | HUVEC VEGF + 5416 - 1 h | VEGF | | 123 | 0 | 0 |
| 325 | HT395 | T | lung | tissue | | none | | 121 | 0 | 184 |
| 162 | DU-145 | T | pro | cell line | prostate carcinoma | none | | 117 | 0 | 15 |
| 128 | RXF 393 | T | renal | cell line | | none | | 117 | 212 | 295 |
| 407 | H1299 - 8 | T | lung | cell line | | 400 ng/ml noco-48 | mutant | 116 | 0 | 0 |
| 455 | SF-268-5 | T | neuro | cell line | | 2 uM AUR2 inhibitor | mutant | 116 | 0 | 0 |
| 360 | OVCAR-4 - 5 | T | OV | cell line | | 2 um AUR2 inhibitor | wt | 115 | 0 | 0 |
| 243 | SK-MEL-2 | T | mel | cell line | Malignant melanoma, metastasis to | none | | 113 | 408 | 119 |
| | | | | | skin of thigh | | | | | |
| 199 | placenta - h | N | plac | tissue | | none | | 113 | 481 | 139 |
| 221 | HT372-normal | N | lung | tissue | | none | | 111 | 0 | 59 |
| 334 | HCT-116 - 4 | T | col | cell line | | 3 mM HU | wt | 110 | 0 | 0 |
| 247 | SK-MEL-28 | T | mel | cell line | Malignant melanomca | none | | 109 | 255 | 0 |
| 125 | SR | T | LEU | cell line | Large Cell leukemia | none | | 108 | 0 | 0 |
| 333 | A549 - 2 | T | lung | cell line | low serum/0.1% FBS | low sereum | wt | 107 | 0 | 0 |
| 477 | Prostate_sampleMG - 3 | unknown | pro | unknown | | unknown | | 106 | 0 | 0 |
| 271 | MK poly A+ | T | unknown | unknown | | unknown | | 105 | 244 | 266 |
| 207 | CRL1441 + TPA (24 h) 8/30 | T | renal | cell line | h lung, embroyonie | TPA | | 104 | 798 | 0 |
| 87 | HT348 | T | pro | tissue | | none | | 103 | 1,459 | 0 |
| 472 | Angio Test1-5 | T | liver | cell line | HapG2 25X DEF-MES for Hypoxia, | 25X DEF-MES | | 102 | 0 | 0 |
| | | | | | 1 h | | | | | |
| 385 | ADR-RES - 8 | T | breast | cell line | | 400 ng/ml noco-48 | mutant | 102 | 0 | 0 |
| 406 | SW480 - 4 | T | col | cell line | | 3 mM HU | mutant | 101 | 0 | 0 |
| 59 | lung - h | N | lung | tissue | | none | | 100 | 0 | 123 |
| 160 | CCRF-CEM | T | LEU | cell line | ALL acute lymphobllastic leukemia | none | | 100 | 0 | 0 |
| 301 | lymph node - h | N | heme | tissue | | none | | 100 | 0 | 144 |
| 152 | SNB-19 | T | neuro | cell line | Glio blastoma | none | | 100 | 0 | 324 |
| 454 | EKVX - 3 | T | lung | cell line | | 200 uM mimosine | mutant | 98 | 0 | 0 |
| 383 | ADR-RES - 7 | T | breast | cell line | | 400 ng/ml noco-24 | mutant | 97 | 00 | 0 |
| 322 | TK-10 | T | renal | cell line | | none | | 97 | 0 | 167 |
| 408 | SW480 - 5 | T | col | cell line | | 2 uM AUR2 inhibitor | mutant | 97 | 0 | 0 |
| 356 | OVCAR-4 - 3 | T | OV | cell line | | 200 uM mimosine | wt | 97 | 0 | 0 |
| 18 | small intestine - h | N | col | tissue | | none | | 96 | 4,224 | 0 |
| 450 | EKVX - 1 | T | lung | cell line | normal/10% FBS | none | mutant | 96 | 0 | 0 |
| 113 | HOP-62 | T | lung | cell line | Lung adenocarcinoma | none | | 95 | 0 | 0 |
| 321 | BioMarker_BS-12 | N | endo | cell line | HUVEC VEGF + 5416 - 6 h | VEGF | | 95 | 389 | 175 |
| 274 | testis - h | N | testes | tissue | | none | | 94 | 0 | 0 |
| 173 | WI-38 72 h | N | lung | cell line | | unknown | | 94 | 310 | 18 |
| 458 | EKVX - 6 | T | lung | cell line | | 400 ng/ml noco-48 | mutant | 94 | 0 | 0 |
| 409 | C33A - 1 | T | cervical | cell line | normal/10% FBS | none | mutant | 94 | 0 | 0 |
| 198 | HCAEC | N | endo | cell line | | none | | 94 | 0 | 0 |
| 95 | HT308 | T | MG | tissue | | none | | 94 | 0 | 0 |
| 302 | RPTEC | N | endo | cell line | mammary epithelial cells | none | | 94 | 398 | 0 |
| 330 | HT218-normal | N | lung | tissue | | none | | 93 | 0 | 57 |
| 467 | Prostate_sampleMG - 20 | unknown | pan | unknown | Hu71 | unknown | | 92 | 0 | 0 |
| 124 | A498 | T | renal | cell line | Kidney carcinoma | none | | 91 | 0 | 0 |
| 335 | A549 - 3 | T | lung | cell line | | 200 uM mimosine | wt | 90 | 0 | 0 |
| 36 | lymph node - h | N | heme | tissue | | none | | 90 | 636 | 0 |
| 182 | HEPM 3d TOFBI detergent + DNase | N | palatal | cell line | TGFBI detergent + Dnase | TGFBI | | 90 | 0 | 0 |
| 16 | skeletal muscle - h | N | muscle | tissue | | none | | 89 | 3,018 | 826 |
| 272 | Spleen - h | N | heme | tissue | | none | | 89 | 0 | 205 |
| 132 | 786-0 | T | renal | cell line | Primary renal cell adenocaricinoma | none | | 89 | 0 | 87 |
| 40 | thymus.h | N | heme | tissue | | none | | 88 | 1,145 | 259 |
| 387 | HeLa - 1 | T | endo | cell line | normal/10% FBS | none | HPV E6 | 88 | 0 | 0 |
| 349 | EKVX - 6 | T | lung | cell line | | 10 uM cisplatin | mutant | 87 | 0 | 0 |
| 56 | HT155 | T | lung | tissue | | none | | 85 | 607 | 108 |
| 396 | ADR-RES - 2 | T | breast | cell line | low serum/0.1% FBS | low sereum | mutant | 84 | 0 | 0 |
| 473 | Prostate_sampleMG - 1 | unknown | pro | unknown | | unknown | | 82 | 0 | 0 |
| 127 | DU-145 | T | pro | cell line | prostate carcinoma | none | | 82 | 0 | 107 |
| 287 | trachea - h | N | trachea | tissue | | none | | 80 | 0 | 46 |
| 309 | brain - h | N | neuro | tissue | | none | | 79 | 58 | 314 |
| 270 | spinal cord - h | N | neuro | tissue | | none | | 78 | 0 | 71 |
| 236 | OVCAE-4 | T | OV | cell line | | none | | 76 | 0 | 9 |
| 449 | SF-268-2 | T | neuro | cell line | low serum/0.1% FBS | low sereum | mutant | 76 | 0 | 0 |
| 189 | MCF-7/ADR-RES | T | breast | cell line | | none | | 74 | 0 | 0 |
| 192 | Duodemum - h | N | col | tissue | | none | | 74 | 0 | 0 |
| 382 | OVCAR-4 - 6 | T | OV | cell line | | 10 uM cisplatin | wt | 74 | 0 | 0 |
| 71 | HT145 | T | MG | tissue | | none | | 72 | 839 | 0 |
| 239 | SN12C | T | neuro | cell line | | none | | 71 | 0 | 120 |
| 428 | U2OS - 3 | T | bone | cell line | | 200 uM mimosine | mutant | 71 | 0 | 0 |
| 190 | Skeletal muscle - h | N | muscle | tissue | | none | | 70 | 0 | 0 |
| 183 | HOS poly A+ | T | bone | cell line | h ostnogenic sarcoma | none | | 69 | 0 | 0 |
| 298 | HT398-normal | N | lung | tissue | | none | | 69 | 0 | 208 |
| 241 | LOX IMVI | T | mel | cell line | Amlanotic malanoma | none | | 67 | 0 | 66 |
| 475 | Prostate_sampleMG - 2 | unknown | pro | unknown | | unknown | | 66 | 0 | 0 |
| 153 | MCF-7/ADR-RES | T | breast | cell line | | none | | 66 | 0 | 206 |
| 138 | Malme-3M | T | renal | cell line | Malignant melanoma, metastasis to | none | | 65 | 104 | 0 |
| | | | | | lung | | | | | |
| 90 | HELA-8 h-031899 | T | endo | cell line | | | | 63 | 1,319 | 0 |
| 62 | HT172 | T | OV | tissue | | none | | 63 | 0 | 274 |
| 386 | MCF-7 - 5 | T | breast | cell line | | 2 uM AUR2 inhibitor | wt | 62 | 0 | 0 |
| 47 | h adult SMC 10/21/92 #17 | T | smc | cell line | | unknown | | 62 | 885 | 0 |
| 497 | Prostate_sampleMG - 17 | T | pro | tissue | metastasis | unknown | | 62 | 0 | 0 |
| 402 | SW480 - 2 | T | col | cell line | low serum/0.1% FBS | low sereum | mutant | 62 | 0 | 0 |
| 398 | ADR-RES - 3 | T | breast | cell line | | 200 uM mimosine | mutant | 61 | 0 | 0 |
| 141 | KM-12 | T | col | cell line | | none | | 60 | 252 | 428 |
| 188 | Pancreas - h | N | pan | tissue | h embryonic palatal mesenchyme | none | | 59 | 177 | 0 |
| 276 | HTB36 24 h TPA RNA 6/23 | T | unknown | cell line | h lung, diploid | TPA | | 59 | 0 | 174 |
| 204 | HT307 | T | MG | tissue | | none | | 58 | 0 | 41 |
| 297 | thymus.h | N | heme | tissue | | none | | 56 | 2,879 | 0 |
| 129 | PC-3 | T | pro | cell line | prostate adenocarcinoma | none | | 55 | 22 | 42 |
| 68 | HT180 | T | lung | tissue | | none | | 54 | 0 | 0 |
| 167 | T-47D | T | breast | cell line | | none | | 54 | 1,052 | 255 |
| 69 | HT143 | T | END | tissue | | none | | 54 | 0 | 117 |
| 120 | MOLT-4 | T | LEU | cell line | ALL Peripheral blood, acute | none | | 52 | 146 | 723 |
| | | | | | lymphoblastic leukemia | | | | | |
| 158 | SF-295 | T | neuro | cell line | | none | | 51 | 0 | 206 |
| 462 | OVCAR-5-8 | T | OV | cell line | | 400 ng/ml noco-48 | mutant | 50 | 0 | 0 |
| 214 | BioMarker_BS-1 | N | endo | cell line | HUVEC control - 0 h | none | | 50 | 534 | 0 |
| 61 | lymph node - h | N | heme | tissue | | none | | 50 | 1,068 | 219 |
| 368 | OVCAR-5 - 1 | T | OV | cell line | normal/10% FBS | none | mutant | 48 | 0 | 0 |
| 75 | CRL1572 3/17/89 | T | OV | cell line | | none | | 47 | 40 | 0 |
| 361 | SF539-2 | T | neuro | cell line | low serum/0.1% FBS | low sereum | wt | 47 | 0 | 0 |
| 166 | 786-0 | T | renal | cell line | Primary renal cell adenocarcinoma | none | | 45 | 791 | 301 |
| 9 | fetal brain - h | N | neuro | tissue | | none | | 42 | 6,215 | 1,666 |
| 06 | U251 | T | neuro | cell line | Glioblastoma | none | | 42 | 294 | 188 |
| 27 | thymus-h | N | heme | tissue | | none | | 42 | 679 | 0 |
| 68 | HT189 | T | OV | tissue | | none | | 41 | 0 | 0 |
| 119 | OVCAR-5 | T | OV | cell line | | none | | 41 | 6 | 0 |
| 495 | Prostate sampleMG - 15 | unknown | neuro | unknown | ES10 primary Ewings tumor | unknown | | 40 | 0 | 0 |
| 51 | fetal liver-h | N | liver | tissue | | none | | 38 | 28 | 0 |
| 169 | Ken-3 | T | lung | cell line | A549 + 50 mg/ml HGF - 6 h | HGF | | 38 | 0 | 218 |
| 57 | kidney - h | N | renal | tissue | | none | | 37 | 0 | 0 |
| 279 | mammary gland - h | N | breast | tissue | | none | | 37 | 0 | 50 |
| 130 | ACHN | T | renal | cell line | Renal adenocarcinoma | none | | 37 | 0 | 107 |
| 136 | LOX IMVI | T | mel | cell line | Amelanotic melanoma | none | | 36 | 911 | 92 |
| 52 | HT288 | T | pan | tissue | | none | | 35 | 479 | 189 |
| 64 | HT178 | T | END | tissue | | none | | 34 | 714 | 0 |
| 275 | bladder - h | N | urinary | tissue | | none | | 34 | 2,586 | 0 |
| 338 | HCT-116 - 6 | T | col | cell line | | 10 μM cisplatin | wt | 33 | 0 | 0 |
| 384 | MCF-7 - 4 | T | breast | cell line | | 3 mM HU | wt | 32 | 0 | 0 |
| 196 | HMEC | N | endo | cell line | coronary artery endothelial cells | none | | 32 | 1,000 | 0 |
| 250 | M14 | T | mel | cell line | Malignant melanoma | none | | 32 | 0 | 0 |
| 294 | HT347 | T | pro | tissue | | none | | 30 | 755 | 0 |
| 320 | RXF 393 | T | renal | cell line | | none | | 30 | 586 | 259 |
| 78 | HT323 | T | MG | tissue | | none | | 28 | 0 | 0 |
| 474 | AngioTest1-6 | T | liver | cell line | HspG2 25X DEF-MES for Hypoxia, | 25X DEF-MES | | 28 | 0 | 0 |
| | | | | | 4 h | | | | | |
| 300 | h adult SMC 10/21/92 #17 | T | smc | cell line | | unknown | | 27 | 0 | 290 |
| 328 | HT157-normal | N | lung | tissue | | none | | 27 | 57 | 133 |
| 427 | Hs68 - 6 | N | lung | cell line | | 10 μM cisplatin | wt | 26 | 0 | 0 |
| 413 | C33A - 3 | T | cervical | cell line | | 200 μM mimosine | mutant | 25 | 0 | 0 |
| 264 | prostate, h | N | pro | tissue | | none | | 25 | 675 | 493 |
| 86 | HELA-A-0h-031899 | T | endo | cell line | | none | | 24 | 410 | 0 |
| 342 | HCT-116 - 8 | T | col | cell line | | 400 ng/ml noco-48 | wt | 22 | 0 | 0 |
| 425 | U2OS - 1 | T | bone | cell line | normal/10% FBS | none | mutant | 22 | 0 | 0 |
| 91 | HT378 | T | lung | tissue | | none | | 21 | 0 | 0 |
| 435 | WI 38 - 2 | N | lung | cell line | low sereum/0.1% FBS | low sereum | wt | 21 | 0 | 0 |
| 303 | NCI-H226 | T | lung | cell line | Lung aquamous ca | none | | 21 | 0 | 0 |
| 209 | bladder - h | N | urinary | tissue | | none | | 19 | 0 | 0 |
| 373 | SF539 - 8 | T | neuro | cell line | | 400 ng/ml noco-48 | wt | 18 | 0 | 0 |
| 228 | EKVX | T | lung | cell line | Lung adenocarcinoma | none | | 17 | 752 | 0 |
| 364 | OVCAR-4 - 7 | T | OV | cell line | | 400 ng/ml noco-24 | wt | 15 | 0 | 0 |
| 440 | Hs68 - 1 | N | lung | cell line | normal/10% FBS | none | wt | 14 | 0 | 0 |
| 461 | SF-268-8 | T | neuro | cell line | | 400 ng/ml noco-48 | mutant | 13 | 0 | 0 |
| 283 | salivary gt. - h | N | salivary | tissue | | none | | 13 | 459 | 0 |
| 424 | H1299 - 5 | T | lung | cell line | | 2 μM AUR2 inhibitor | mutant | 13 | 0 | 0 |
| 133 | HCT 116 | T | col | cell line | Colon carcinoma | none | | 12 | 0 | 138 |
| 205 | CCL137 RNA 3/21/88 | N | lung | cell line | | none | | 12 | 0 | 110 |
| 208 | Ken-1 | T | lung | cell line | A549 + 50 ng/ml HGF - 0h | none | | 12 | 0 | 0 |
| 240 | OVCAR-8 | T | OV | cell line | | none | | 12 | 0 | 173 |
| 72 | HT227 | T | kidney | tissue | | none | | 11 | 0 | 0 |
| 401 | SW480 - 1 | T | col | cell line | normal/10% FBS | none | mutant | 11 | 0 | 0 |
| 370 | OVCAR-5 - 3 | T | OV | cell line | | 200 μM mimosine | mutant | 10 | 0 | 0 |
| 291 | HEPM 3d untreated | N | palatal | cell line | pulmonary artery endobelial cells | none | | 9 | 630 | 11 |
| 420 | H1299 - 3 | T | lung | cell line | | 200 μM mimosine | mutant | 8 | 0 | 0 |
| 479 | Prostate_sampleMG - 4 | unknown | pro | unknown | | unknown | | 7 | 0 | 0 |
| 273 | liver - h | N | liver | tissue | | none | | 6 | 0 | 110 |
| 329 | HT213-normal | N | kidney | tissue | | none | | 5 | 279 | 0 |
| 392 | MCF-7 - 8 | T | breast | cell line | | 400 ng/ml noco-48 | wt | 5 | 0 | 0 |
| 180 | thymus - h | N | heme | tissue | | none | | 5 | 62 | 0 |
| 268 | small intestine - h | N | col | tissue | | none | | 4 | 0 | 0 |
| 486 | AngioTest1-12 | N | endo | cell line | HUVEC 30 mm stimulation with | bFGF | | 2 | 0 | 0 |
| | | | | | bFGF | | | | | |
| 355 | HT29 - 7 | T | col | cell line | | 400 ng/ml noco-24 | mutant | 2 | 0 | 0 |
| 485 | Prostate_sampleMG - 8 | unknown | pro | unknown | | unknown | | 1 | 0 | 0 |
| 12 | prostate, h | N | pro | tissue | | none | | 0 | 4,960 | 699 |
| 176 | HT398 | T | lung | tissue | | none | | 0 | 0 | 590 |
| 232 | A546/ATCC | T | lung | cell line | Lung carcinoma | none | | 0 | 3,434 | 572 |
| 159 | MDA-MB-435 | T | breast | cell line | | none | | 0 | 0 | 561 |
| 234 | OVCAR-3 | T | OV | cell line | Ovary adenocarcinoma | none | | 0 | 0 | 470 |
| 317 | HT151 | T | lung | tissue | | none | | 0 | 1,571 | 439 |
| 164 | HCT116 | T | col | cell line | Colon carcinoma | none | | 0 | 895 | 403 |
| 142 | SK-MEL-5 | T | mel | cell line | Malignant melanoma, metastasis to | none | | 0 | 0 | 318 |
| | | | | | axillary node | | | | | |
| 117 | OVCAR-4 | T | OV | cell line | | none | | 0 | 0 | 308 |
| 54 | HT139 | T | END | tissue | | none | | 0 | 0 | 302 |
| 137 | COLO 205 | T | col | cell line | Colon adenocarcinoma | none | | 0 | 232 | 297 |
| 179 | HT372 | T | lung | tissue | | none | | 0 | 644 | 275 |
| 49 | fetal kidney - h | N | renal | tissue | | none | | 0 | 0 | 262 |
| 102 | SK-OV-3 | T | OV | cell line | Ovary adenocarcinoma, malignant | none | | 0 | 0 | 254 |
| | | | | | ascites | | | | | |
| 219 | h keratinocytes 2/25/92 #10 | T | keratiocyte | cell line | h neuroblastoma | unknown | | 0 | 0 | 252 |
| 110 | SF-268 | T | neuro | cell line | Glioblastoma | none | | 0 | 0 | 252 |
| 206 | WI-38 72h 0.5% FBS, 24h 10% FBS | T | lung | cell line | 0.5% FBS, 24 h 10% FBS | low serum | | 0 | 74 | 208 |
| 99 | EKVX | T | lung | cell line | Lung adenocarcinoma | none | | 0 | 0 | 180 |
| 163 | T-47D | T | breast | cell line | | none | | 0 | 1,556 | 172 |
| 94 | HELA-11h-O31899 | T | endo | cell line | | | | 0 | 977 | 170 |
| 157 | MDA-MB-231 | T | breast | cell line | Brest adenocarcinoma, pleural | none | | 0 | 0 | 142 |
| | | | | | effusion | | | | | |
| 39 | Heart - h | N | heart | tissue | h choriocarcinoma | none | | 0 | 0 | 116 |
| 171 | BioMarker_BS-5 | N | endo | cell line | HUVEC VEGF - 1 h | VEGF | | 0 | 95 | 116 |
| 184 | Salivery gi. - h | N | salivary | tissue | | none | | 0 | 0 | 104 |
| 233 | SR | T | LEU | cell line | Large Cell leukemia | none | | 0 | 0 | 93 |
| 282 | prostate, h | N | pro | tissue | | none | | 0 | 0 | 89 |
| 289 | uterus - h | N | uterus | tissue | | none | | 0 | 0 | 85 |
| 178 | HT281 | T | MG | tissue | | none | | 0 | 0 | 81 |
| 170 | BioMarker_BS-3 | N | endo | cell line | HUVEC control - 6 h | none | | 0 | 434 | 79 |
| 262 | HTB10 | T | neuro | cell line | h ovarian teratocarcinoma ascitic | none | | 0 | 0 | 77 |
| | | | | | fluid cells | | | | | |
| 188 | HT311 | T | MG | tissue | | none | | 0 | 0 | 75 |
| 194 | TCGP | T | testes | tissue | | none | | 0 | 0 | 67 |
| 248 | K-562 | T | LEU | cell line | CML Chronic myslogenous | none | | 0 | 0 | 67 |
| | | | | | leukemia | | | | | |
| 244 | SK-OV-3 | T | OV | cell line | Ovary adenocarcinoma, malignant | none | | 0 | 0 | 62 |
| | | | | | ascites | | | | | |
| 193 | WISH (Collagen) poly A+ | T | unknown | cell line | h amnion. HaLa markers | none | | 0 | 409 | 60 |
| 149 | M14 | T | mel | cell line | Malignant melanoma | none | | 0 | 2,528 | 58 |
| 112 | SF-295 | T | neuro | cell line | | none | | 0 | 463 | 56 |
| 327 | HT157 | T | lung | tissue | | none | | 0 | 0 | 52 |
| 284 | HELA-EXP-031899 | T | endo | cell line | | | | 0 | 0 | 48 |
| 48 | h fibroblasts 3/31/92 #12 | T | fibroblast | cell line | metastasis to supraoribital area | unknown | | 0 | 0 | 36 |
| 181 | MDA-N | T | breast | cell line | | none | | 0 | 0 | 35 |
| 252 | MCF7 | T | breast | cell line | Breast adenocarcinoma, pleural | none | | 0 | 0 | 23 |
| | | | | | effusion | | | | | |
| 88 | HELA-6h-031899 | T | endo | cell line | | | | 0 | 719 | 21 |
| 58 | HT163 | T | OV | tissue | | none | | 0 | 270 | 19 |
| 172 | BioMarker_BS-9 | N | endo | cell line | HUVEC 5416 - 6 h | SU5416 | | 0 | 0 | 14 |
| 73 | HT302 | T | pan | tissue | | none | | 0 | 0 | 4 |
| 3 | bone marrow - h | N | bone | tissue | | none | | 0 | 2,984 | 0 |
| 175 | 781T untreated + Dnase | T | unknown | cell line | | none | | 0 | 1,604 | 0 |
| 285 | thyroid gland - h | N | thyroid | tissue | | none | | 0 | 1,301 | 0 |
| 210 | Ken-2 | T | lung | cell line | A549 + 50 ng/ml HGF - 1 h | HGF | | 0 | 903 | 0 |
| 235 | HCT-15 | T | col | cell line | Colon adenocarcinoma | none | | 0 | 680 | 0 |
| 84 | BioMarker_BS-4 | N | endo | cell line | HUVEC control - 24 h | none | | 0 | 601 | 0 |
| 293 | HTB36 Oh RNA | T | unknown | cell line | | none | | 0 | 572 | 0 |
| 316 | KM-12 | T | col | cell line | | none | | 0 | 511 | 0 |
| 203 | HT160 | T | lung | tissue | | none | | 0 | 502 | 0 |
| 266 | skeletal muscle - h | N | muscle | tissue | | none | | 0 | 423 | 0 |
| 258 | HPAEC | N | endo | cell line | renal proximal tubule epithelial cells | none | | 0 | 420 | 0 |
| 109 | NCI-H522 | T | lung | cell line | Lung adenocarcinoma | none | | 0 | 413 | 0 |
| 200 | fetal liver - h | N | liver | tissue | | none | | 0 | 234 | 0 |
| 174 | CRL1441 RNA 8/30 | T | renal | cell line | | none | | 0 | 171 | 0 |
| 253 | HT392-normal | N | lung | tissue | | none | | 0 | 167 | 0 |
| 267 | MNNG-OS poly A+ | T | bone | cell line | h osteosarcoma, chem. transf. | none | | 0 | 45 | 0 |
| 213 | HT370 | T | lung | tissue | | none | | 0 | 16 | 0 |
| 280 | pancreas - h | N | pan | tissue | | none | | 0 | 0 | 0 |
| 389 | HeLa - 3 | T | endo | cell line | | 200 uM mimosine | HPV E6 | 0 | 0 | 0 |
| 391 | HeLa - 4 | T | endo | cell line | | 3 mM HU | HPV E6 | 0 | 0 | 0 |
| 393 | HeLa - 5 | T | endo | cell line | | 2 uM AUR2 inhibitor | HPV E6 | 0 | 0 | 0 |
| 397 | HeLa - 7 | T | endo | cell line | | 400 ng/ml noco-24 | HPV E6 | 0 | 0 | 0 |
| 463 | HeLa - 2 | T | endo | cell line | low serum/0.1% FBS | low serum | HPV E6 | 0 | 0 | 0 |
| 344 | HT29 - 1 | T | col | cell line | normal/10% FBS | none | mutant | 0 | 0 | 0 |
| 346 | HT29 - 2 | T | col | cell line | low serum/0.1% FBS | low sereum | mutant | 0 | 0 | 0 |
| 347 | EKVX - 5 | T | lung | cell line | | 2 uM AUR2 inhibitor | mutant | 0 | 0 | 0 |
| 348 | HT29 - 3 | T | col | cell line | | 200 uM mimosine | mutant | 0 | 0 | 0 |
| 351 | EKVX - 7 | T | lung | cell line | | 400 ng/ml noco-24 | mutant | 0 | 0 | 0 |
| 352 | HT29 - 5 | T | col | cell line | | 2 uM AUR2 inhibitor | mutant | 0 | 0 | 0 |
| 368 | OVCAR-5 - 2 | T | OV | cell line | low sereum/0.1% FBS | low serum | mutant | 0 | 0 | 0 |
| 394 | ADR-RES - 1 | T | breast | cell line | normal/10% FBS | none | mutant | 0 | 0 | 0 |
| 400 | ADR-RES - 4 | T | breast | cell line | | 3 mM HU | mutant | 0 | 0 | 0 |
| 405 | H1299 - 7 | T | lung | cell line | | 400 ng/ml noco-24 | mutant | 0 | 0 | 0 |
| 414 | SW480 - 8 | T | col | cell line | | 400 ng/ml noco-48 | mutant | 0 | 0 | 0 |
| 415 | C33A - 4 | T | cervical | cell line | | 3 mM HU | mutant | 0 | 0 | 0 |
| 417 | C33A - 5 | T | cervical | cell line | | 2 uM AUR2 inhibitor | mutant | 0 | 0 | 0 |
| 419 | C33A - 6 | T | cervical | cell line | | 10 uM cisplatin | mutant | 0 | 0 | 0 |
| 421 | C33A - 7 | T | cervical | cell line | | 400 ng/ml noco-24 | mutant | 0 | 0 | 0 |
| 422 | H1299 - 4 | T | lung | cell line | | 3 mM HU | mutant | 0 | 0 | 0 |
| 430 | U2OS - 4 | T | bone | cell line | | 3 mM HU | mutant | 0 | 0 | 0 |
| 432 | U2OS - 5 | T | bone | cell line | | 2 uM AUR2 inhibitor | mutant | 0 | 0 | 0 |
| 434 | U2OS - 6 | T | bone | cell line | | 10 uM cisplatin | mutant | 0 | 0 | 0 |
| 438 | U2OS - 8 | T | bone | cell line | | 400 ng/ml noco-48 | mutant | 0 | 0 | 0 |
| 448 | SF-268-1 | T | neuro | cell line | normal/10% FBS | none | mutant | 0 | 0 | 0 |
| 331 | A549 - 1 | T | lung | cell line | normal/10% FBS | none | wt | 0 | 0 | 0 |
| 332 | HCT-116 - 3 | T | col | cell line | | 200 uM mimosine | wt | 0 | 0 | 0 |
| 336 | HCT-116 - 5 | T | col | cell line | | 2 uM AUR2 inhibitor | wt | 0 | 0 | 0 |
| 339 | A549 - 5 | T | lung | cell line | | 2 uM AUR2 inhibitor | wt | 0 | 0 | 0 |
| 340 | HCT-116 - 7 | T | col | cell line | | 400 ng/ml noco-24 | wt | 0 | 0 | 0 |
| 341 | A549 - 6 | T | lung | cell line | | 10 uM cisplatin | wt | 0 | 0 | 0 |
| 343 | A549 - 7 | T | lung | cell line | | 400 mg/ml noco-24 | wt | 0 | 0 | 0 |
| 345 | A549 - 8 | T | lung | cell line | | 400 mg/ml noco-48 | wt | 0 | 0 | 0 |
| 353 | HCT-116 - 1 | T | col | cell line | normal/10% FBS | none | wt | 0 | 0 | 0 |
| 363 | SF539 - 3 | T | neuro | cell line | | 200 uM mimosine | wt | 0 | 0 | 0 |
| 365 | SF539 - 4 | T | neuro | cell line | | 3 mM HU | wt | 0 | 0 | 0 |
| 367 | SF539 - 5 | T | neuro | cell line | | 2 uM AUR2 inhibitor | wt | 0 | 0 | 0 |
| 374 | OVCAR-4 - 1 | T | OV | cell line | normal/10% FBS | none | wt | 0 | 0 | 0 |
| 376 | OVCAR-4 - 2 | T | OV | cell line | low serum/0.1% FBS | low sereum | wt | 0 | 0 | 0 |
| 390 | MCF-7 - 7 | T | breast | cell line | | 400 ng/ml noco-24 | wt | 0 | 0 | 0 |
| 429 | Hs68 - 7 | N | lung | cell line | | 400 ng/ml noco-24 | wt | 0 | 0 | 0 |
| 431 | Hs68 - 8 | N | lung | cell line | | 400 ng/ml noco-48 | wt | 0 | 0 | 0 |
| 437 | WI38 - 3 | N | lung | cell line | | 200 uM mimosine | wt | 0 | 0 | 0 |
| 439 | WI38 - 4 | N | lung | cell line | | 3 mM HU | wt | 0 | 0 | 0 |
| 443 | WI38 - 6 | N | lung | cell line | | 10 uM cisplatin | wt | 0 | 0 | 0 |
| 444 | Hs68 - 3 | N | lung | cell line | | 200 uM mimosine | wt | 0 | 0 | 0 |
| 445 | WI38 - 7 | N | lung | cell line | | 400 ng/ml noco-24 | wt | 0 | 0 | 0 |
| 446 | Hs68 - 4 | N | lung | cell line | | 3 mM HU | wt | 0 | 0 | 0 |
| 447 | WI 38 - 8 | N | lung | cell line | | 400 ng/ml noco-48 | wt | 0 | 0 | 0 |
| 460 | HCT-116 - 2 | T | col | cell line | low serum/0.1% FBS | low sereum | wt | 0 | 0 | 0 |
| 488 | OVCAR-4 - 8 | T | OV | cell line | | 400 ng/ml noco-48 | wt | 0 | 0 | 0 |
| 42 | Fetal brain - h | N | neuro | tissue | | none | | 0 | 0 | 0 |
| 69 | HT368 | T | lung | tissue | | none | | 0 | 0 | 0 |
| 96 | HELA-12h-031899 | T | endo | cell line | | | | 0 | 0 | 0 |
| 100 | IGROV1 | T | OV | cell line | | none | | 0 | 0 | 0 |
| 131 | HCC-2998 | T | col | cell line | | none | | 0 | 0 | 0 |
| 134 | TK-10 | T | renal | cell line | | none | | 0 | 0 | 0 |
| 147 | UACC-257 | T | mel | cell line | Malignant melanoma | none | | 0 | 0 | 0 |
| 150 | NCI-H522 | T | lung | cell line | Lung adenocarcinoma | none | | 0 | 0 | 0 |
| 177 | HT140 | T | kidney | tissue | | none | | 0 | 0 | 0 |
| 186 | testis - h | N | testes | tissue | | none | | 0 | 0 | 0 |
| 191 | UTOS (Mundy) poly a+ | T | bone | cell line | | none | | 0 | 0 | 0 |
| 211 | HT369 | T | lung | tissue | | none | | 0 | 0 | 0 |
| 212 | Ken-4 | T | lung | cell line | A549+50 ug/ml HGF - 24 h | HGF | | 0 | 0 | 0 |
| 217 | HT377 | T | lung | tissue | | none | | 0 | 0 | 0 |
| 220 | BioMarker_BS-7 | N | endo | cell line | HUVEC VEGF - 24 h | VEGF | | 0 | 0 | 0 |
| 224 | BioMarker_BS-10 | N | endo | cell line | HUVEC 5416 - 24 h | SU5416 | | 0 | 0 | 0 |
| 226 | HOP-92 | T | lung | cell line | Lung large cell carcinoma | none | | 0 | 0 | 0 |
| 230 | NCI-H23 | T | lung | cell line | Lung adenocarcinoma | none | | 0 | 0 | 0 |
| 231 | RPMI 8226 | T | LEU | cell line | Multiple myeloma | none | | 0 | 0 | 0 |
| 238 | OVCAR-5 | T | OV | cell line | | none | | 0 | 0 | 0 |
| 242 | IGROV1 | T | OV | cell line | | none | | 0 | 0 | 0 |
| 246 | SF-539 | T | neuro | cell line | Glioblastoma | none | | 0 | 0 | 0 |
| 249 | UACC-257 | T | mel | cell line | Malignant melanoma | none | | 0 | 0 | 0 |
| 255 | HT279 | T | MG | tissue | | none | | 0 | 0 | 0 |
| 260 | bone marrow - h | N | heme | tissue | | none | | 0 | 0 | 0 |
| 292 | HT149 - normal | N | lung | tissue | | none | | 0 | 0 | 0 |
| 299 | Fetal brain - h | N | neuro | tissue | | none | | 0 | 0 | 0 |
| 311 | cerebellum - h | N | neuro | tissue | | none | | 0 | 0 | 0 |
| 315 | HT218 | T | lung | tissue | | none | | 0 | 0 | 0 |
| 471 | Prostate_sampleMG - 22 | unknown | pan | unknown | Hu399 | unknown | | 0 | 0 | 0 |
| 480 | AngioTest1-9 | N | endo | cell line | HUVEC 30 mn stimulation with | VEGF | | 0 | 0 | 0 |
| | | | | | VEGF | | | | | |
| 481 | Prostate_sampleMG - 5 | unknown | pro | unknown | | unknown | | 0 | 0 | 0 |
| 482 | AngioTest1-10 | N | endo | cell line | HUVEC 30 mn stimulation with | PDGF | | 0 | 0 | 0 |
| | | | | | PDGF | | | | | |
| 484 | AngioTest1-11 | N | endo | cell line | HUVEC 10 mn stimulation with | bFGF | | 0 | 0 | 0 |
| | | | | | bFGF | | | | | |
| 487 | Prostate_sampleMG - 9 | unknown | pro | unknown | | unknown | | 0 | 0 | 0 |
| 494 | Prostate_sampleMG - 14 | unknown | neuro | unknown | ES7 primary Ewings tumor | unknown | | 0 | 0 | 0 |
| 496 | Prostate_sampleMG - 16 | T | pro | tissue | metastasis | unknown | | 0 | 0 | 0 |
| 498 | Prostate_sampleMG - 18 | T | pro | tissue | metastasis | unknown | | 0 | 0 | 0 |
| 499 | Prostate_sampleMG - 19 | T | pro | tissue | metastasis | unknown | | 0 | 0 | 0 |

Table 6, “Multiple Tissue Blot”, contains results of probing a Clontech Multiple Tissue Blot with radioactively labeled probes derived from SGP002 and SGP012. The table list the issues on the blot and the values obtained for relative gene expression in each tissue.

TABLE 6
Multiple Tissue Blot CIP02

| Tissue | ID#NA11_SGP012 | ID#NA2_SGP002 |
|---|---|---|
| whole brain | 1244 | 14946 |
| cerebellum left | 3610 | 22681 |
| substantia nigra | 0 | 14730 |
| heart | 0 | 14816 |
| esophagus | 2008 | 15554 |
| colon, transverse | 1607 | 20564 |
| kidney | 44 | 25345 |
| lung | 637 | 27317 |
| liver | 68 | 37568 |
| leukemia, HL-60 | 0 | 2204 |
| fetal brain | 0 | 7572 |
| cerebral cortex | 1178 | 16874 |
| cerebellum, right | 5201 | 35351 |
| accumbens nucleus | 0 | 14985 |
| aorta | 203 | 13539 |
| stomach | 0 | 22332 |
| colon, descending | 3812 | 16311 |
| skeletal muscle | 220 | 20600 |
| placenta | 497 | 64169 |
| pancreas | 264 | 19531 |
| HeLa S3 | 0 | 20564 |
| fetal heart | 649 | 15777 |
| frontal lobe | 0 | 11984 |
| corpus callosum | 1972 | 27350 |
| thalamus | 789 | 22702 |
| atrium, left | 465 | 14405 |
| duodenum | 695 | 20940 |
| rectum | 0 | 12642 |
| spleen | 0 | 18882 |
| bladder | 528 | 22077 |
| adrenal gland | 570 | 138400 |
| leukemia, K562 | 0 | 7331 |
| fetal kidney | 620 | 38826 |
| parietal lobe | 492 | 21242 |
| amygdala | 830 | 14740 |
| pituitary gland | 1620 | 41283 |
| atrium, right | 754 | 8285 |
| jujenum | 2358 | 21596 |
| thymus | 54 | 29593 |
| uterus | 1427 | 18077 |
| thyroid gland | 65 | 25540 |
| leukemia, MOLT-4 | 92 | 8081 |
| fetal liver | 1189 | 29080 |
| occipital lobe | 449 | 17070 |
| caudate nucleus | 334 | 22638 |
| spinal cord | 556 | 6385 |
| ventricle, left | 0 | 9420 |
| iteum | 1002 | 15704 |
| peripheral blood leukocyte | 1435 | 15521 |
| prostate | 0 | 46589 |
| salivary gland | 741 | 45205 |
| Burkitt's lymphoma, Raji | 0 | 2497 |
| fetal spleen | 0 | 24452 |
| temperal lobe | 913 | 15048 |
| hippocampus | 608 | 13826 |
| ventricle, right | 811 | 9938 |
| ilocecum | 0 | 18970 |
| lymph node | 3497 | 23227 |
| testis | 10751 | 33336 |
| mamary gland | 2429 | 43077 |
| Burkitt's | 2439 | 2384 |
| lymphoma, Daudi | | |
| fetal thymus | 797 | 31519 |
| paracentral gyrus | 0 | 16294 |
| cerebral cortex | | |
| medulla oblongata | 730 | 18935 |
| inter-ventricular septum | 0 | 18269 |
| appendix | 0 | 23931 |
| bone marrow | 1127 | 10289 |
| ovary | 437 | 7103 |
| colorectal adeno- | 466 | 15172 |
| carcinoma, SW480 | | |
| fetal lung | 1 | 26587 |
| pons | 875 | 12156 |
| putamen | 0 | 27800 |
| apex of the heart | 311 | 9897 |
| colon, ascending | 1409 | 12683 |
| trachea | 1894 | 22056 |
| lung carcinoma, A549 | 1276 | 15151 |

### EXAMPLES

The examples below are not limiting and are merely representative of various aspects and features of the present invention. The examples below demonstrate the isolation and characterization of the serine/threonine phosphatases of the invention.

### Example 1

### Identification and Characterization of Protein Phosphatase Genes from Genomic DNA

Materials and Methods

Novel phosphatases were identified from the Celera human genomic sequence databases, and from the public Human Genome Sequencing project (http://www.ncbi.nlm.nih.gov/) using hidden Markov models (HMMRs). The genomic database entries were translated in six open reading frames and searched against the model using a Timelogic Decypher box with a Field programmable array (FPGA) accelerated version of HMMR2.1. The DNA sequences encoding the predicted protein sequences aligning to the HMMR profile were extracted from the original genomic database. The nucleic acid sequences were then clustered using the Pangea Clustering tool to eliminated repetitive entries. The putative protein phosphatase sequences were then sequentially run through a series of queries and filters to identify novel protein phosphatase sequences. Specifically, the HMMR identified sequences were searched using BLASTN and BLASTX against a nucleotide and amino acid repository containing known human protein phosphatases and all subsequent new protein phosphatase sequences as they are identified. The output was parsed into a spreadsheet to facilitate elimination of known genes by manual inspection. Two models were developed, a “complete” model and a “partial” or Smith Waterman model. The partial model was used to identify sub-catalytic phosphatase domains, whereas the complete model was used to identify complete catalytic domains. The selected hits were then queried using BLASTN against the public nrna and EST databases to confirm they are indeed unique. In some cases the novel genes were judged to be orthologues of previously identified rodent or vertebrate protein phosphatases.

Many of the sequences filed in the provisional patents did not contain the entire coding sequence. Extension of partial DNA sequences to encompass the full-length open-reading frame was carried out by several methods. Iterative blastn searching of the cDNA databases listed in Table 7 was used to find cDNAs that extended the genomic sequences. “LifeGold” databases are from Incyte Genomics, Inc (http://www.incyte.com/). NCBI databases are from the National Center for Biotechnology Information (http:/www.ncbi.nlm.nih.gov/). All blastn searches were conducted using a blosum62 matrix, a penalty for a nucleotide mismatch of −3 and reward for a nucleotide match of 1. The gapped blast algorithm is described in: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

Extension of partial DNA sequences to encompass the full-length open-reading frame was also carried out by iterative searches of genomic databases. The first method made use of the Smith-Waterman algorithm to carry out protein-protein searches of the closest homologue or orthologue to the partial. The target databases consisted of Genescan and open-reading frame (ORF) predictions of all human genomic sequence derived from the human genome project (HGP) as well as from Celera. The complete set of genomic databases searched is shown in Table 8, below. Genomic sequences encoding potential extensions were further assessed by blastp analysis against the NCBI nonredundant to confirm the novelty of the hit. The extending genomic sequences were incorporated into the cDNA sequence after removal of potential introns using the Seqman program from DNAStar. The default parameters used for Smith-Waterman searches were as shown next Matrix: blosum 62; gap-opening penalty: 12; gap extension penalty: 2. Genescan predictions were made using the Genescan program as detailed in Chris Burge and Sam Karlin “Prediction of Complete Gene Structures in Human Genomic DNA”, JMB (1997) 268(1):78-94). ORF predictions from genomic DNA were made using a standard 6-frame translation.

Another method for defining DNA extensions from genomic sequence used iterative searches of genomic databases through the Genescan program to predict exon splicing. These predicted genes were then assess to see if they represented “real” extensions of the partial genes based on homology to related phosphatases.

Another method involved using the Genewise program (http://www.sanger.ac.uk/Software/Wise2/) to predict potential ORFs based on homology to the closest orthologue/homologue. Genewise requires two inputs, the homologous protein, and genomic DNA containing the gene of interest. The genomic DNA was identified by blastn searches of Celera and Human Genome Project databases. The orthologs were identified by blastp searches of the NCBI non-redundant protein database (NRAA). Genewise compares the protein sequence to a genomic DNA sequence, allowing for introns and frameshifting errors.

TABLE 7
Databases used for cDNA-based sequence extensions

| Database | Database Date |
|---|---|
| LifeGold templates | Oct 2000 |
| LifeGold compseqs | Oct 2000 |
| LifeGold compseqs | Oct 2000 |
| LifeGold compseqs | Oct 2000 |
| LifeGold fl | Oct 2000 |
| LifeGold flft | Oct 2000 |
| NCBI human Ests | Oct 2000 |
| NCBI murine Ests | Oct 2000 |
| NCBI nonredundant | Oct 2000 |

TABLE 8
Databases used for genomic-based sequence extensions

| | Number of | |
|---|---|---|
| Database | entries | Database Date |
| Celera v. 1-5 | 5,306,158 | Jan 19/00 |
| Celera v. 6-10 | 4,209,980 | Mar 24/00 |
| Celera v. 11-14 | 7,222,425 | Apr 24/00 |
| Celera v. 15 | 243,044 | May 14/00 |
| Celera v. 16-17 | 25,885 | Apr 04/00 |
| Celera Assembly 5 (R1.25) | 3,313 | Oct 13/00 |
| Celera Assembly 4 (R1.24) | 636,234 | Aug 28/00 |
| Celera Assembly 3 (R 1.22, | 1,132,900 | Jul 21/00 |
| 1.23) | | |
| HGP Phase 0 | 4,944 | May 04/00 |
| HGP Phase 1 | 28,478 | May 05/00 |
| HGP Phase 2 | 1,508 | May 04/00 |
| HGP Phase 3 | 9,971 | May 05/00 |
| HGP Phase 0 | 3,189 | Nov 1/00 |
| HGP Phase 1 | 20,447 | Nov 1/00 |
| HGP Phase 2 | 1,619 | Nov 1/00 |
| HGP Phase 3 | 9,224 | Nov 1/00 |
| HGP Chromosomal | 2759 | Aug 1/00 |
| assemblies | | |

Results:

The sources for the sequence information used to extend the genes in the provisional patents are listed below. For genes that were extended using Genewise, the accession numbers of the protein ortholog and the genomic DNA are given. (Genewise uses the ortholog to assemble the coding sequence of the target gene from the genomic sequence). The amino acid sequences for the orthologs were obtained from the NCBI non-redundant database of proteins (http://www.ncbi.nlm.nih.gov/Entrez/protein.html). The genomic DNA came from two sources: Celera and NCBI-NRNA, as indicated below. cDNA sources are also listed below. Abbreviations: HGP: Human Genome Project; NCBI, National Center for Biotechnology Information.

SGP006 (SEQ ID NO:1)

The N-terminal region (1-335) was derived from Genewise predictions using Celera contig 300825903, with protein homologs gi|7242951, gi|8923483 and gi|6714641. Genscan predictions of Celera contig 300825903 was also used. NCBI ESTs used to extend sequence: BE793092.1, gi|9127446, gi|5927364, gi|8148569, gi|9096610, gi|10214290, gi|5927365, gi|4533101, gi|1948748, gi|2010582, gi|30571, gi|2433225, gi|8152915. Icyte sequence 339266.1 is missing exon 7 (GFSVSTAGRMHIFKPVSVQAMW). Public sequence gi|7242951 (KIAA1298) is missing exon 11 and starts near the beginning of axon 10. The lack of exon 11 causes a frameshift, and so KIAA1298 has a divergent N-terminal predicted peptide, reading exon 10 in a different frame. SGP006 is identical to KIAA1298 over the C-terminal 715 amino acids of SGP006 (amino acids 335 to 1049).

SGP006 (SEQ ID NO:1) is 6374 nucleotides long. The open reading frame starts at position 34 and ends at position 3183, giving an ORF length of 3150 nucleotides. The predicted protein is 1049 amino acids long. This sequence is full length (start methionine to stop codon). It is classified as (superfamily/group/family): Dual Phosphatase, DSP, MKP. This gene maps to chromosomal position 12q21.3-q22. Amplification of this chromosomal position has been associated with the following human diseases: Bladder carcinoma (12q21-q24, 1/16) (Knuutila, et al.). This gene contains candidate single nucleotide polymorphisms at the following positions: 6222=R (ccaaacataagtggcacar) dbSNP|rs881179_allele. ESTs for this gene in the public domain (dbEST) are: BE793092.1, AI651213.1, BE256978.1. This gene has repetitive sequence at the following nucleotide positions: Alu 5750-6010; 5750-5770.

SGP002 (SEQ ED NO:2)

SGP002 nucleic acid sequence was derived from Genewise algorithm run with Celera genomic DNA 70000016592596 and the protein homolog gi_{—}6679156. A similar Genscan prediction gave an N-terminal extension, and comparison with HGP contig gi|7658297 corrected a frameshift in the genewise prediction. Close homologs are of same length. NCBI ESTs gi|7950699 and gi|760983 extend into 5′ and 3′ UTRs respectively. Genomic sequence was used to correct sequence errors in these ESTs. NCBI EST gi|10717958 encodes a splice variant. Incyte EST 1026659.2 encodes an alternative splice form missing an exon which includes part of the phosphatase domain. Incyte EST 1026659.7 adds further 172 nucleotides of 5′ UTR to the gene. Incyte and public ESTs show expression in many tissues, most commonly digestive system, nervous system, respiratory system, and male and female genitalia.

SGP002 (SEQ ID NO:2) is 2732 nucleotides long. The open reading frame starts at position 538 and ends at position 2535, giving an ORF length of 1998 nucleotides. The predicted protein is 665 amino acids long. This sequence is full length (start methionine to stop codon). It is classified as (superfamily/group/family): Dual Phosphate, DSP, MKP. This gene maps to chromosomal position 12p11.1-p12.1. This chromosomal position has been associated with the following human diseases: Testis cancer (12p11.2-p12.1, 10/11); non-small cell lung cancer (12p11.2-p12, 4/50), and breast carcinoma (12p11-pter, 2/36) (Knuutila, et al.). ESTs for this gene in the public domain (dbEST) are: BE897795. This gene has repetitive sequence at the following nucleotide positions: 2610-2631.

SGP001 (SEQ ID NO:3)

Used genscan, and genewise with Celera contig 5000012164505, and protein homologs gi_{—}6714641 and gi_{—}7242951. Several public and Incyte ESTs were used to extend the gene, using genomic data to correct for EST sequence errors. They were: Incyte sequences: 210343.1, 210343.2, 637331CB1; and NCBI ESTs: gi|3894502, gi|1100172, gi|11100172, gi4137370, gi|650571, gi|6885171, gi|1123262, and gi|6590412.

SGP001 (SEQ ID NO:3) is 2260 nucleotides long. The open reading frame starts at position 709 and ends at position 2205, giving an ORF length of 1497 nucleotides. The predicted protein is 498 amino acids long. This sequence is full length (start methionine to stop codon). It is classified as (superfamily/group/family): Dual Phosphatase, DSP, MKP. This gene maps to chromosomal position Xp11.1-11.3. This chromosomal potion has been associated with the following human diseases: Prostate cancer (Xp11-q13, 1/9) and small cell lung cancer (Xp11.2, 1/13) (Knuutila, et al.). ESTs for this gene in the public domain (dbEST) are: AI272231, BF206586. This gene has repetitive sequence at the following nucleotide positions: 579-598.

SGP018 (SEQ ID NO:4)

The sequence for SGP018 is predicted from Celera contig 68000017706859, using Genscan and genewise with gi_{—}7305011 and gi_{—}7705959. The genewise prediction covered most of a putative phosphatase. The Genscan prediction overlapped and extended the genewise predictions, and almost all of the genscan was covered by ESTs from Incyte and dbEST. In all cases, ESTs were corrected by first aligning genomic (Celera/HGP) sequence. A splice variant predicted by Genscan would replace the sequence SEFLDEALLTYR with YCHYIIFSCVFIS (changes the nt sequence ACTGTCATTACATCATTTTCTCTTGTGTTTTCATTTC to CTGAGTTCCTGGATGAGGCGCTGCTGACTTACAG. EST origins: Incyte sequences: 981712.1, 981712.3, 981712.2, 364575.1, 061688.1, 144608.1, 7668648H1, 7473603CB1, 7473604CB1. Public ESTS, including: gi|6880197, gi|6880191, gi|6880141, gi|5441204, gi|5441149, gi|1242174, gi|0984357. Genscan also predicts an alternative C-terminus, where the sequence from VHLL to the C-terminus is replaced by

ANGNSVRSTSRFSSSSTREGREMHKFSRSTYNETSSSREESPEPYFFRRT
PESSEREESPEPQRPWARSRDWEDVEESSKSDFSEFGAKRKFTQSFMSEE
EGEKERTENREEGRFASGRRSQYRRSNDREEEEMDDEAIIAAWRRRQEET
RTKLQKRRED.

The cDNA sequence from 544-612 is not covered by any ESTs. Accordingly, the upstream and downstream sequences could be different genes and a start at position 613 would give a peptide a later start, at MLESAE; this would give a protein with good homology and the same N-terminal length has the closest mouse homolog, PTP13. A possible alternative splice form seem by comparing incyte ESTs 061688.1 and 7668648H1 predicts a protein form which is missing the Nterminus and instead starts the sequence MTPEK.

SGP018 (SEQ ED NO:4) is 4361 nucleotides long. The open reading frame starts at position 208 and ends at position 3609, giving ORF length of 3402 nucleotides. The predicted protein is 1133 amino acids long. This sequence is full length (start methionine to stop codon). It is classified as (superfamily/group/family): Dual Phosphatase DSP, MKP. This gene has not been mapped to a chromosomal position. This gene contains candidate single nucleotide polymorphisms at the following positions: 2929=M (agaagatgtctgagtacm) dbSNP|ss1765941; 1161=S (catctaccccaatgas) dbSNP|ss1765940. ESTs for this gene in the public domain (dbEST) are: BF114881. This gene has repetitive sequence at the following nucleotide positions: 1603-1627.

SGP003 (SEQ ID NO:5)

SGP003 sequence is derived from Genewise with Celera contig 173000019613519 and NCBI homolog template gi_{—}7705959, extended to the stop codon by genomic walk. The cDNA template is built from 4 EST clones, 2 from muscle, one each from bone and parathyroid gland. Corrected a frameshift in the sequence using HGP contig gi|10178266, and further extended the sequence by 5′ walking the genomic until the first stop. SGP003 has a 235 nucleotide open reading frame preceding the start codon, extending from nucleotide 3 to nucleotide 239, shown in capital letters below:

caAGGGTTTCAGGTCGCACTGGAAAATCATTTTGCAAGCAGATGT
CATAGGTCTCCTCTTAGACTGGACGGCACGCAAGGTCAGCGTCACAGATC
TGACCCTAAAAATAGGCCTCTGTTGCCAGTCGGGGTGGCTGGGCGTGCGG
CTGCTACATGCCCCACGGACCAGAACCTCCCGACGCGGCCAGGCCCCGGC
ACACCCAGCTGCAGAAAGGAGAGAAAATCCCTTGGCTCTAAAatg

This open reading fame codes for the following peptide sequence:

QGFQVALENHFASRCHRSPLRLDGTQGQRHRSDPKNRPLLPVGVAGRAAA
TCPTDQNLPTRPGPGTPSCRKERKSLGSK

The start codon at position 240 conforms to the Kozak rule for initiating methionines, having an A at the −3 position.

SGP003 (SEQ ID NO:5) is 1262 nucleotides long. The open reading frame starts at position 240 and ends at position 902, giving an ORF length of 663 nucleotides. The predicted protein is 220 amino acids long. This sequence is full length (start methionine to stop codon). It is classified as (superfamily/group/family): Dual Phosphatase, DSP, MKP. This gene maps to chromosomal position CHR10. This gene has repetitive sequence at the following nucleotide positions: 311-334.

SGP014 (SEQ ID NO:6)

Sequence for SGP014 was built from Celera contig 92000005033031 using genscan and genewise, with protein homolog templates gi_{—}7293532, gi_{—}7705959 and gi_{—}9502074. The predicted genewise/genscan proteins were extended by overlaps with several ESTs from dbEST (AA723271, AW444890.1, AA435513.1), and confirmed the public sequence gi|7705959. The full predicted peptide is 549 AA, with full DSP domains from 37-181 and 368-541. The following NCBI ESTs come from this gene: gi|11105857, gi|1998334, gi|1423340, gi|2186481, gi|6986652, gi|10372533, gi|2186305, gi|4825880, gi|2740908, gi|3213953, gi|2436350, gi|2140427, gi|2833919, gi|5768154, gi|1134009, gi|2046580, gi|4822411, gi|11152927. The following Incyte Sequences come from this gene: 128077.1, 1384255.1, 8009838H1, 304421CB1. Alternative splicing is very prevalent. The individual exons are as follows: a parenthetical AA at the end of an exon is a residue which crosses the exons at least in the FL form:

>Exon1:
MAETSLPELGGEDKATPCPSILELEELLRAGKSSCSRVDEVWPNLFIGD
(A)
>Exon2:
ATANNRFELWKLGITHVLNAAHKGLYCQGGPDFYGSSVSYLGVPAHDLPD
FDISAYFSSAADFIHRALNTPG (A)
>Exon3:
KVLVHCVVGVSRSATLVLAYLMLHQRLSLRQAVITVRQHRWVFPNRGFLH
QLCRLD (H)
>Exon4:
WSLLPAMGLCHFATLALILLVLLEALAQADTQKMVEAQRGVGPRACYSIW
LLLAPTPPLSHCLQSPQ
>Exon5:
KQHQVCGDRRLKASSTNCPSEKCTAWARYSHRW
>Exon6:
AHILVPLKIQLRRVPDSFSQQMPETSYLTRVGPDIQCWPESW (G)
>Exon7:
MDSLQKQDLRRPKIHGAVQASPYQPPTLASLQRLLWVRQAATLNHIDEVW
PSLFLGD (A)
>Exon8:
YAARDKSKLIQLGITHVVNAAAGKFQVDTGAKFYRGMSLEYYGIEADDNP
FFDLSVYFLPVARYIRAALSVPQ (E)
>Exon9:
DGHGCLFFPKGWVVQGQVADAKLVLPTGRVLVHCAMGVSRSATLVLAFLM
ICENMTLVEAIQTVQAHRNICPNSGFLRQLQVLDNRLGRETGRF

DSP domain 1 runs from the second half of Exon1 to the end of exon3, domain 2 runs from towards the end of exon7 to almost the end of exon 9. Alternative splicing shown by ESTs: Start of exon 9 (EDG-LPT) is missing in gi|6986652, gi|2186305, gi|10372533 is missing the end of exon 8 and the beginning of exon 9 (KFQ-LPT), gi|11105857 is missing exons 2, 3, 4, 6, and the beginning of exon 9 (EDG-GRV). It has a frameshift between exon 1 and 5, which may be a sequencing error, gi|2186481 is missing exons 2, 3, 6. gi|2740908, gi|2436350, gi|2140427, gi2833919 have a frameshift relative to the consensus towards the end of exon 9, which replaces the sequence after NSGF with SGSSRFWTTDWGGRRGGSDLAGSQDP*. This change destroys the end of the phosphatase domain, and is not similar to anything in the database. It could be due to genomic polymorphism between individuals, a repeated sequencing error, or possibly some form of gene regulation. These ESTs come from testis (2, same library), prostate and cardiac, so are not a library artifact 8009838H1 has an internal deletion within exon 2 from YLG-SSA. 304421CB1 is missing exons 2-6 and has a frameshift between exons 1 and 7, and is missing start of exon 9. 128077.1 is missing exons 2,3,6 and the start of exon 9.

SGP014 (SEQ ID NO:6) is 1917 nucleotides long. The open reading frame starts at position 31 and ends at position 1680, giving an ORF length of 1650 nucleotides. The predicted protein is 549 amino acids long. This sequence is full length (start methionine to stop codon). It is classified as (superfamily/group/family): Dual Phosphatase, DSP, MKP. This gene maps to chromosomal position 10q21.3. This chromosomal position has been associated with the following human diseases: Squamous cell carcinomas of the head and neck (10q21-q22, 2/30) (Knuutila, et al.). ESTs for this gene in the public domain (dbEST) are: AA723271, AW444890.1, AA435513.1.

SGP060 (SEQ ID NO:7)

The sequence of SGP060 is derived from Genewise, using Celera contig 6514035_{—}1 and protein homolog NP_{—}057448. NCBI ESTs used to extend the sequence include BF207232, BF314818, AW953216.1.

SGP060 (SEQ ID NO:7) is 636 nucleotides long. The open ring frame starts at position 1 and ends at position 636, giving an ORF length of 636 nucleotides. The predicted protein is 211 amino acids long. This sequence is full length (start methionine to stop codon). It is classified as (superfamily/group/family): Dual Phosphatase, DSP, MKP. This gene maps to chromosomal position 8p11.1-q11.1 centromeric. This chromosomal position has been associated with the following human diseases: breast carcinoma (8p11-p12, 8/53); non-small cell lung cancer (18p11.2, 2/50) (Knuutila, et al.). ESTs for this gene in the public domain (dbEST) are: BF207232, BF314818, AW953216.1.

SGP008 (SEQ ID NO:8)

Genscan and genewise were done on Celera contig 78000006091415, using homologs gi|9910432, gi|7294466 and gi|7298988. These were verified and extended with public ESTs gi|7280554, gi|6925677 and gi|6142140, and Incyte sequence 7475576CB1. The predicted cDNA was corrected using sequence from the Celera contig and current HGP contigs. Comparison with non-human ESTs and public protein sequences indicate that there may be an internal start to the protein, at amino acid position 95 (at MGNG).

SGP008 (SEQ ID NO:8) is 1326 nucleotides long. The open reading fire starts at position 1 and ends at position 990, giving an ORF length of 990 nucleotides. The predicted protein is 329 amino acids long. This sequence is full length (start methionine to stop codon). It is classified as (superfamily/group/family): Dual Phosphatase, DSP, STYX. This gene maps to chromosomal position 20q11.2. This gene contains candidate single nucleotide polymorphisms at the following positions: 871=S (cagcagcctccgagggaaccs) dbSNP|ss1389419. ESTs for this gene in the public domain (dbEST) are: AW406620.1, BF377364.1, AW593296.1. This gene has repetitive sequence at the following nucleotide positions: 1251-1270.

SGP039 (SEQ D NO:9)

SGP039 is derived from Celera sequence 17000030279756, and from Incyte sequences 272616.1 and 7476908CB1.

SGP039 (SEQ ID NO:9) is 1083 mucleotides long. The open reading frame starts at position 1 and ends at position 1083, giving an ORF length of 1083 nucleotides. The predicted protein is 360 amino acids long. This sequence is full length (start methionine to stop codon). It is classed as (superfamily/group/family): Serine Phosphatase, STP, PP2C. This gene has not been mapped to a chromosomal position. ESTs for this gene in the public domain (dbEST) are: BE147139.

SGP040 (SEQ ED NO:10)

The sequence for SGP040 is derived from Celera sequence 17000091609039 and the public sequence NM_{—}018444.1 for pyruvate dehydrogenase phosphatase.

SGP040, PDP (SEQ ID NO:10) is 1725 nucleotides long. The open reading frame starts at position 1 and ends at position 1725, giving an ORF length of 1725 nucleotides. The predicted protein is 574 amino acids long. This sequence is full length (start methionine to stop codon). It is classified as (superfamily/group/family): Serine Phosphatase, STP, PP2C. This gene maps to chromosomal position 8q21.3. This chromosomal position has been associated with the following human diseases: Mantle cell lymphoma (18q21-q23; 5/50) (Knuutila, et al.). ESTs for this gene in the public domain (dbEST) are: AV706533.1, AV705571.1, AV710801.1.

SGP012 (SEQ ID NO:11)

The sequence for SGP012 is derived from Genewise, using Celera sequences 94000002120453; 142000016367225; 142000016006753, as genomic DNA input and NP_{—}031981 (murine PTP-EST) as protein homolog. Incyte ESTSs that overlap this sequence include 1005303.1, and 7109651_{—}3. Public ESTs which overlap with the sequence include AL042532.1, AI381571, and AW872677.

SGP012 PTP-ESP (SEQ ID NO:11) is 4719 nucleotides long. The open reading frame starts at position 1 and ends at position 4719, giving an ORF length of 4719 nucleotides. The genomic sequence for this gene is of fairly poor quality, i.e., it has not been assembled and has apparent sequence errors. Thus the nucleic acid and protein sequences are partial, with gaps indicated by “X” s in the sequence. The predicted protein is 1573 amino acids long. This sequence contains the catalytic domain. It is classified as (superfamily/group/family): Tyrosine Phosphatase, RPTP, PTPd. This gene has not been mapped to chromosomal position. ESTs for this gene in the public domain (dbEST) are: AL042532.1, AI381571, AW872677. This gene has repetitive sequence at the following nucleotide positions: 1305-1324.

SGP024 (SEQ ID NO:12)

SGP024 is derived from Genewise using Celera DNA sequence 142000016226692 as geneomic source and NP_{—}002830.1 (human PTP delta) as protein homolog.

SGP024 (SEQ ID NO: 12) is 354 nucleotides long. The open reading frame starts at position 1 and ends at position 357, giving an ORF length of 357 nucleotides. The predicted protein is 118 amino acids long. This sequence is a partial catalytic domain. It is classified as (superfamily/group/family): Tyrosine Phosphatase, Receptor PTP, PTPdelta sub-family.

### Example 2

### Predicted Proteins

SGP006, KIAA1298 (SEQ ID NO:1) encodes SEQ ID NO:13, a protein that is 1049 Amino acids long. It is classified as an MKP. The phosphatase domain in this protein matches the hidden Markov profile for a MKP/DSP phosphatase from profile position 1 to profile position 173 (full length catalytic domain). The position of the catalytic region within the encoded protein is from amino acid 308 to amino acid 446. The results of a Smith Waterman search of the public database of amino acid sequences (NRAA) with this protein sequence yielded the following results. The C-terminus of SGP006 (amino acid positions 322 to 1049) is 100% identical to KIAA1298 protein [Homo sapiens]. The output can be summarized as follows: P-value=0; number of identical amino acids=715; percent identity=100%; percent similarity=100%; the accession number of the most similar entry in NRAA is BAA92536.1; the name or description, and species, of the most similar protein in NRAA is: KIAA1298 protein [Homo sapiens]. The region N-terminal to this identity with KIAA1298 is novel—for amino acids 120 to 477, the results of a Smith Waterman search of the public database of amino acid sequences (NRAA) yielded the following results: P-value=1.50E-99; number of identical amino acids=248; percent identity=46%; percent similarity=59%; the accession number of the most similar entry in NRAA is BAA89534.1; the name or description, and species, of the most similar protein in NRAA is: MAP kinase phosphatase [Drosophila melanogaster]. The N-terminal sequence of SGP006, from amino acid 1 to 263, is also novel, and the results of a Smith Waterman search of the public database of amino acid sequences (NRAA) with this protein sequence yielded the following results: P-value=6.80E-58; number of identical amino acids=119; percent identity=41%; percent similarity=59%; the accession number of the most similar entry in NRAA is NP_{—}060327.1; the name or description, and species, of the most similar protein in NRAA is: Hypothetical protein FLJ20515 [Homo sapiens].

SGP002 (SEQ ID NO:2) encodes SEQ ID NO:14, a protein that is 665 amino acids long. It is classified as an MKP. The phosphatase domain in this protein matches the hidden Markov MKP/DSP phosphatase domain from profile position 1 to profile position 173 (full length catalytic domain). The position of the catalytic region within the encoded protein is from amino acid 158 to amino acid 297. The results of a Smith Waterman search of the public database of amino acid sequences (NRAA) with this protein sequence yielded the following results: P-value=1.10E-157; number of identical amino acids=304; percent identity=46%; percent similarity=60%; the accession number of the most similar entry in NRAA is NP_{—}004411.1; the name or description, and species, of the most similar protein in NRAA is: dual specificity phosphatase 8 [Homo sapiens]. This protein contains a Rhodanese-like domain (amino acids 11 to 131). The rhodanese domain has been associated with thiosulfate: cyanide sulfurtransferase (EC 2.8.1.1) activity. The presence of this domain may indicate that SGP002 is regulated in response to the cellular redox environment (Nandi et al., Int J Biochem Cell Biol April 2000; 32(4):465-73; Rhodanese as a thioredoxin oxidase).

SGP001 (SEQ ID NO:3) encodes SEQ ID NO: 15, a protein that is 498 amino acids long. It is classified as an MKP. The phosphatase domain in this protein matches the hidden Markov profile for a MKP/DSP phosphatase domain from profile position 1 to profile position 173. The position of the catalytic region within the encoded protein is from amino acid 307 to amino acid 441. The results of a Smith Waterman search of the public database of amino acid sequences (NRAA) with this protein sequence yielded the following results: Pscore=8.30E-133; number of identical amino acids=250; percent identity=47%; percent similarity=60%; the accession number of the most similar entry in NRAA is BAA89534.1; the name or description, and species, of the most similar protein in NRAA is: MKP [Drosophila melanogaster].

SGP018 (SEQ ID NO:4) encode SEQ ID NO:16, a protein that is 1133 amino acids long. It is classified as an MKP. The phosphatase domain in this protein matches the hidden Markov profile from profile position MKP/DSPphosphatase domain from profile position 1 to profile position 173. The position of the catalytic region within the encoded protein is from amino acid 185 to amino acid 330. The results of a Smith Waterman search of the public database of amino acid sequence (NRAA) with this protein sequence yielded the following results: P-value=2.20E-27; number of identical amino acids=79; percent identity=45%; percent similarity=63%; the accession number of the most similar entry in NRAA is NP_{—}057448.1; the name or description, and species, of the most simiar protein in NRAA is: Protein phosphate LOC51207 [Homo sapiens].

SGP003 (SEQ ID NO:5) encodes SEQ ID NO: 17, a protein that is 220 amino acids long. It is classified as an MKP. The phosphatase domain in this protein matches the hidden Markov profile from profile position MKP/DSPphosphatase domain from profile position 1 to profile position 173. The position of the catalytic region within the encoded protein is from amino acid 54 to amino acid 199. The results of a Smith Waterman search of the public database of amino acid sequences (NRAA) with this protein sequence yielded the following results: P-value=3.40E-54; number of identical amino acids=91; percent identity=49%; percent similarity=68%; the accession number of the most similar entry in NRAA is NP_{—}057448.1; the name or description, and species, of the most similar protein in NRAA is: Protein phosphatase LOC51207 [Homo sapiens].

SGP014 (SEQ ID NO:6) encodes SEQ ID NO: 18, a protein that is 549 amino acids long, with two phosphatase domains. Both domains in this protein match the hidden Markov profile for an MKP/DSP phosphatase profile from position 1 to profile position 173 (full length). Both DSP domains are similar, with best hits to gi|7705959 (human; partial of this gene), and DSP13 from mouse. The position of the catalytic regions within the encoded protein are from amino acid 37 to amino acid 181 for the N-terminal domain, and from 368 to 520 for the C-terminal domain The results of a Smith Waterman search of the public database of amino acid sequences (NRAA) with this protein sequence yielded the following results: for amino acid 324-549, P-value=7.50E-122; number of identical amino acids=198; percent identity=88%; percent similarity=88%; the accession number of the most similar entry in NRAA is NP_{—}057448.1; the name or description, and species, of the most similar protein in NRAA is: Protein phosphatase LOC51207 [Homo sapiens]. For amino acids 1-198, the results of a Smith Waterman search of the public database of amino acid sequences yielded the following results: P-value=8.20E-36; number of identical amino acids=75; percent identity=45%; percent similarity=65%; the accession number of the most similar entry in NRAA is NP_{—}004081.1; the name or description, and species, of the most similar protein in NRAA is: Dual specificity phosphatase 3 [Homo sapiens].

SGP060 (SEQ ID NO:7) encodes SEQ ID NO:19, a protein that is 211 amino acids long. It is classified as an MKP. The phosphatase domain in this protein matches the hidden Markov profile for MKP/DSP phosphatase from profile position 1 to profile position 173 (full length). The position of the catalytic region within the encoded protein is from amino acid 61 to amino acid 204. The results of a Smith Waterman search of the public database of amino acid sequences (NRAA) with this protein sequence yielded the following results: P-value=1.10E-48; number of identical amino acids=86; percent identity=53%; percent similarity=72%; the accession number of the most similar entry in NRAA is NP_{—}057448.1; the name or description, and species, of the most similar protein in NRAA is: Protein phosphatase LOC51207 [Homo sapiens].

SGP008 (SEQ ID NO:8) encodes SEQ ID NO:20, a protein that is 329 amino acids long. It is classified as an MKP/STYX. The phosphatase domain in this protein matches the hidden Markov profile from profile position MKP/DSPphosphatase domain from profile position 1 to profile position 173. The position of the catalytic region within the encoded protein is from amino acid 98 to amino acid 235. The results of a Smith Waterman search of the public database of amino acid sequences (NRAA) with this protein sequence yielded the following results: P-value=4.40E-172; number of identical amino acids=260; percent identity=92%; percent smilarity=92%; the accession number of the most similar entry in NRAA is CAC10008.1; the name or description, and species, of the most similar protein in NRAA is: Novel protein [Homo sapiens].

SGP039 (SEQ ID NO:9) encodes SEQ ID NO:21, a protein that is 360 amino acids long. It is classified as: PP2C. The phosphatase domain in this protein matches the hidden Markov profile from profile position 1 to profile position 301 (Full length catalytic). The position of the catalytic region within the encoded protein is from amino acid 91 to amino acid 344. The results of a Smith Waterman search of the public database of amino acid sequences (NRAA) with this protein sequence yielded the following results: P-value=1.00E-106; number of identical amino acids=164; percent identity=98%; percent similarity=99%; the accession number ofthe most similar entry in NRAA is AAD17235.1; the name or description, and species, of the most similar protein in NRAA is: PP 2C [Mus musculus].

SGP040, PDP (SEQ ID NO: 10) encodes SEQ ID NO:22, a protein that is 574 amino acids long. It is classified as: PP2C. The phosphatase domain in this protein matches the hidden Markov profile from position 1 to position 301. The position of the catalytic region within the encoded protein is from amino acid 209 to amino acid 497. The results of a Smith Waterman search of the public database of amino acid sequences (NRAA) with this protein sequence yielded the following results: P-value=0; number of identical amino acids=574; percent identity=100%; percent similarty=100%; the accession number of the most similar entry in NRAA is NP_{—}060914.1; the name or description, and species, of the most similar protein in NRAA is: Pyruvate dehydrogenase phosphatase [Homo sapiens].

SGP012 PTP-ESP (SEQ D NO:11) encodes SEQ D NO:23, a protein that is 1573 amino acids long. It is classified as: PTP, delta phosphatase-like. The phosphatase domain in this protein matches the hidden Markov profile for a PTP phosphatase, from profile position 1 to profile position 264 (full length catalytic). The position of the catalytic region within the encoded protein is from amino acid 1010 to 1259. The results of a Smith Waterman search of the public database of amino acid sequences (NRAA) with this protein sequence yielded the following results: P-value=0; number of identical amino acids=1053; percent identity=60%; percent similarity=70%; the accession number of the most similar entry in NRAA is NP_{—}031981.1; the name or description, and species, ofthe most similar protein in NRAA is: Embryonic stem cell phosphatase [Mus musculus]. This protein contains five fibronectin domains at: amino acid positions 35-120; 128-208; 390-471; 484558; 668-748. Gaps with the sequence are indicted by “XXX”.

SGP024 (SEQ ID NO:12) encodes SEQ ID NO:24, a protein that is 118 amino acids long. It is classified as a PTP, related to PTP delta. The phosphatase domain in this protein matches the hidden Markov profile for a PTP from profile position 205 to profile position 264 (this is a partial catalytic domain, representing the C-terminal region). The position of the catalytic region within the encoded protein is from amino acid 3 to amino acid 58. The results of a Smith Waterman search of the public database of amino acid sequences (NRAA) with this protein sequence yielded the following results: P-value=5.90E-54; number of identical amino acids=90; percent identity=76%; perfect similarity=82%; the accession number of the most similar entry in NRAA is CAA38068.1; the name or description, and species, of the most similar protein in NRAA is: Protein-tyrosine phosphatase delta [Homo sapiens].

### Example 3

### Expression Analysis of Novel Mammalian Protein Phosphatases

The gene expression patterns for selected genes were studied using two techniques: 1) a tissue microarray developed at Sugen, containing 499 tissues and probed with labeled genes; and 2) a commercial array of tissue from Clontech, probed with labeled genes.

1) Tissue Arrays

“cDNA libraries” derived from a variety of sources were immobilized onto nylon membranes and probed with 32P-labeled cDNA fragments derived from the gene(s) of interest. The sources of RNA are listed in Table 3. They are: 1) Biochain Institute (Hayward, Calif.; http://www.biochain.com/main_{—}3.html): 2) Clontech (Palo Alto, Calif., http://www.clontech.com/): 3) mammalian cell lines used by the National Cancer Institute (NCI) Developmental Therapeutics Program (http://dtp.nci.nih.gov/; can be ordered from ATCC: http://www.atcc.org.catalogs.html; 4) PathAssociates (http://www.saic.com/company/subsidiaries/pai.html; San Diego, Calif.). The protocols for preparing cDNA arrays are detailed below. Several cell lines were treated with compounds to evaluate their effects on gene expression. There were eight treatments: 1) control, 2) low sereum, 3) 200 uM mimosine, 4) 3 mM HU, 5) 2 uM AUR2 inhibitor, 6) 10 uM cisplatin, 7) 400 ng/ml nocodozole-24 hours, and 8) 400 ng/ml nocodozole-48 hours. The treated cell lines are listed by cell line name followed by a number from 1 to 8.

1.

“cDNA libraries” derived from over 450 tissue or cell line sources were immobilized onto nylon membranes and probed with 32P-labeled cDNA fragments derived from the gene(s) of interest. To make the cDNA, total RNA or mRNA was used as template in a reverse transcription reaction to generate single-stranded cDNAs (ss cDNA) that were tagged with specific sequences at each end. An oligo dT primer containing a specific sequence (CDS: AAGCAGTGGTAACAACGCAGAGTACT₃₀VN (V=A,G,C N=A,G,C,T)) anneals at the polyA track at the 3′ end of the mRNA and the reverse transcriptase (MMLV RnaseH⁻) transcribes the antisense strand until it reaches the end of the RNA strand when it adds additional C residues. If a primer (SMII: AAGCAGTGGTAACAACGCAGAGTACGCGGG or ML2G AAGTGGCAACAGAGATAACGCGTACGCGGG) ending with 3 Gs is added, it anneals to the added Cs and the MMLV recognizes the rest of the primer sequence as template and continues transcription. As a result, the synthesized cDNAs contain specific sequence tags at both the 5′ and the 3′ end. When the 5′ and the 3′ ends are tagged with the same sequence (CDS and SMII) it is referred to as “symmetric”. When the 5′ end is tagged with a different sequence than the 3′ end (CDS and ML2G) is referred to as “asymmetric”. A double-stranded “cDNA library” is then generated by PCR amplification using the 3′PCR and ML2 primers (3′ PCR: AAGCAGTGGTAACAACGCAGAGT and ML2: AAGTGGCAACAGAGATAACGCGT) that anneal to the added sequence tags.

The amplified “cDNA libraries” were manually arrayed onto nylon membranes with a 384 pin replicator. The DNA was denatured by alkali treatment, neutralized and cross-linked by WV light. The arrays were pre-hybridized with Express Hyb (Clontech) and hybridized with ³²P labeled probes generated by random hexamer priming of cDNA fragments corresponding to the genes of interest After washing, the blots were exposed to phosphorimaging cassettes and the intensity of the signal was quantified. The amount of the DNA on the arrays was also quantified by treating non-denatured or denatured arrays with Syber Green I or Syber Green II respectively (1:100,000 in 50 mM Tris, pH8.0) for 2 minutes. After washing with 50 mM Tris, pH8.0, the fluorescent emission was detected with a phosphorimager (Molecular Dynamics) and quantified. The amount of the arrayed DNA was used to normalize the hybridization signal and the corrected values are tabulated in Table 5.

Statistical Methods:

The tissue array data for the 3 phosphatases were standardized for statistical analysis across the different tissue types using range standardization. Standardization converts measurements to a common scale. We used range standardization, which subtracts the smallest value of each variable from each value and divides by its range. The new scale starts at 0 and ends at 1.0. The following statistical procedures were implemented on the standardized data: generation of descriptive statistics, graphical visualization, hierarchical and k-means cluster analysis (at 10, 7, and 5 clusters), and comparison of groups using analysis of variance (ANOVA). When tissue-specific data were present for both normal and tumor samples, the two groups were directly compared for fold differences. All statistical analyses were carried out separately for the symmetric and asymmetric tissue array laboratory methods because we know from experience with past data that gene expression is dependent upon the method used. All statistical analyses were carried out using SYSTAT 9.01 (Copyright© 1999 by SPSS, Inc.).

### SUMMARY OF RESULTS

TABLE 9
Fold difference in mean expression between normal
human tissue and cancer cell lines, and between
and normal tissue and tumor samples.

| | | | Normal vs. Tumor | | |
|---|---|---|---|---|---|
| | | Tissue vs. | | Within Cell | Within |
| CDNA | Gene | Cell Line | Pooled | Line | Tissue |
| Symmetric | SGP003 | 23.02 | 28 | 6.26 | 10.92 |
| Symmetric | SGP060 | 2.86 | 2.67 | −1.92 | 0 |
| Symmetric | SGP018 | 2.25 | 2.33 | 2.4 | −1.32 |
| ASymmetric | SGP003 | +2.33** | 1.37 | 1.19 | −1.03 |
| ASymmetric | SGP060 | +2.38**** | 1.08 | −2.06 | −1.26 |
| ASymmetric | SGP018 | 1.01 | 1.51*** | −1.43* | −1.72** |

*(ANOVA F, p ≦ 0.1),
**(ANOVA F, p ≦ 0.05),
***(ANOVA F, p ≦ 0.01),
****(ANOVA F, p ≦ 0.0001)
Symmetric: [n = 41 (tissue), 66 (cell line), 4 (normal cell line), 62 (tumor cell line), 40 (normal tissue), and 1 (tumor tissue)].
Asymmetric: [n = 112 (tissue), 262 (cell line), 43 (normal cell line), 219 (tumor cell line), 49 (normal tissue), and 63 (tumor tissue)].

Discussion

1. SGP003 (SEQ ID NO:5)

This gene was observed to express consistently higher in tissue samples (as versus cell-line samples) and in normal samples (as versus tumor samples) in both the symmetric and asymmetric methods. We observed much higher fold differences in the symmetric method than in the asymmetric method (Table 9), but because of inadequate sample size and large variation in the data, we did not find the difference to be statistically significant in the symmetric method. On the other hand, the fold difference of 2.33 between the tissue and cell-line samples in the asymmetric method was statistically significant at p<0.05. Because this phosphatase is expressed higher in normal than in tumor samples, it may play a role in tumor suppression. Highest levels of expression of this gene were observed in the normal samples, particularly those drawn from brain, fetal brain, fetal kidney, and glandular tissues such as the pituitary and adrenal glands. We did observe some relatively high levels of expression in a few tumor samples (lymphoblastoma, neuroblastoma, melanoma, lung, colon, breast, and renal tumors). Selected clusters and their rankings according to levels of expression for this phosphatase are listed below:

(Symmetric Data)

Cluster ranking by highest mean expression:
Cluster 1 (singleton). NORMAL GROUP: heart sample (tissue).
Cluster 2 (singleton). NORMAL GROUP: spinal cord (tissue).
Cluster 3 (8 members). NORMAL GROUP only: colon (stomach tissue), colon (small intestinal tissue), mammary epithelial cells (cell line), spleen (heme tissue), lymph node (heme tissue), fetal lung (tissue), fetal brain (neural issue), and prostate (tissue).

(Asymmetric Data)

Cluster ranking by highest mean expression:
Cluster 1 (singleton). NORMAL GROUP: adrenal gland (tissue).
Cluster 2 (3 members). NORMAL GROUP: thymus (heme tissue). TUMOR GROUP: lung carcinoma (cell line), and a neuro sample (tissue).
Cluster 3 (14 members). NORMAL GROUP: thyroid gland (tissue), lymph node (heme tissue), and coronary artery endothelial cells (cell line); and TUMOR GROUP: lung (tissue), malignant melanoma metastasis to lung (cell line), breast (cell line), unknown (cell line), breast (cell line), HNS (tissue), endothelial (cell line), endothelial (cell line), prostate (tissue), kidney (tissue), and renal adenocarcinoma (cell line).
   2. SGP060 (SEQ ID NO:7)

The highest expressers in the asymmetric method were tumor samples. Although they represented different types of tumors, we observed consistently very high expression in various lung cancer samples. This gene may be an oncogene important in lung cancer. In normal tissues, it expressed highest in brain tissue samples and in fetal kidney. Selected clusters and their rankings according to levels of expression for this phosphatase are listed below:

Cluster ranking by highest mean expression (Asymmetric data):
Cluster 1 (2 members). TUMOR GROUP only: lung (tissue) and lung carcinoma (cell line).
Cluster 2 (3 members). TUMOR GROUP: lung (tissue) and ovary adenocarcinoma (cell line); and NORMAL GROUP: prostate (tissue).
Cluster 3 (3 members). TUMOR GROUP only: lung (tissue), neuroblastoma (tissue), and colon carcinoma (cell line).
Cluster 4 (8 members). TUMOR GROUP: MG (tissue), smc (cell line), glioblastoma (cell line), lung large cell carcinoma (cell line), END (tissue), primary renal cell carcinoma (cell line), and lung (tissue); and NORMAL GROUP: brain (tissue).
Cluster 5 (10 members). TUMOR GROUP: lung (tissue), malignant melanoma metastasis to lung (cell line), colon adenocarcinoma (cell line), renal (cell line), unknown sample (MK ploy A+), breast (cell line), renal primary clear cell carcinoma metastasizing (cell line), ovary (tissue), and neuroblastoma (keratiocyte cell line); and NORMAL GROUP: fetal kidney (tissue).
   3. SGP018 (SEQ ED NO:4)

According to the asymmetric method, this gene expresses higher in tumor samples (as versus the normal samples) and this pattern was consistent and statistically significant for pooled, within tissue, and within cell-line samples (Table 9). This gene expresses very highly across a broad range of tumor types, and may be particularly important in glioblastoma and ovarian cancer. Like KAP, this phosphatase may be a good target as a marker and in therapeutics.

Cluster ranking by highest mean expression (Asymmetric data):
Cluster 1 (singleton). TUMOR GROUP: neuro (tissue).
Cluster 2 (3 members). TUMOR GROUP only: HNS (tissue), renal adenocarcinoma (cell line), and ovary (cell line).
Cluster 3 (5 members). TUMOR GROUP only: malignant melanoma, metastasis to lung (cell line), colon (cell line, treated with 3 mM HU), ovary (cell line, treated with 10 uM cisplatin), neuro (cell line, treated with 10 uM cisplatin), and PML peripheral blood, promyelocytic leukemia (cell line).
Cluster 4 (11 members). TUMOR GROUP: colon (cell line, treated with 10 uM cisplatin), breast (cell line), endothelial cells (cell line, treated with HeLa25X DEF-MES for hypoxia, 4 hours), unknown sample (unknown), cervical (cell line, treated with 400 ng/ml noco-48 hours), kidney (tissue), lung (tissue), lung (tissue), endothelial cells (cell line), and lung (tissue); and NORMAL GROUP: HUVEC (cell line, treated with 10 mn PDGF stimulation).

Cluster 5 (27 members). TUMOR GROUP: kidney carcinoma (cell line), lung (issue), neuro (cell line, treated with 10 uM cisplatin), lung (tissue), bone (cell line), breast (cell line), lung (tissue), lung (cell line, treated with 3 mM HU), neuro (cell line, treated with 400 ng/ml noco-24 hours), endothelial cells (cell line, treated with HeLa25X DEF-MES for hypoxia, 0 hours), ovary (cell line, treated with 2 uM AUR2 inhibitor), breast (cell line, treated with normal/10% FBS), breast (cell line, treated with 2 uM AUR2 inhibitor), breast (cell line, treated with 200 uM mimosine), bone (cell line, treated with low serim/0.1% FBS), colon (cell line, treated with 10 uM cisplatin), cervical (cell line, treated with low serim/0.1% FBS), endothelial cells (cell line), kidney (tissue), pancreas (tissue), and renal (cell line); NORMAL GROUP: endothelial cells (cell line, treated with HUVEC VEGF+5416-24 hours), lung (tissue), endothelial cells (cell line, HUVEC unstimulated/control), and stomach (colon tissue).

2) Multiple Tissue Expression Blots (MTE)

MTE (Multiple Tissue Expression) blots were obtained from Clontech Laboratories, Inc (see table 6). These blots contained 84 arrayed cDNA samples derived from normal human tissue and human cell lines, and controls. The expression blots were prehybridized with ExpressHyb hybridization solution (Clontech Laboratories) containing 0.1 mg/ml denatured salmon sperm DNA at a temperature of 65° C. for two hours. Radioactive DNA probes were prepared using the Random Priming DNA labeling kit (Roche). Purified DNA fragments (100 ng) were labeled with 250 uCi of 32P-labeled dCTP for 45 minutes using the kit protocol. Unincorporated nucleotide was removed through the use of a spin column (ProbeQuant G50 micro columns, Amersham Pharmacia, Inc.). After denaturation by boiling for three minutes, the probe was introduced into the prehybridization solution, and the blot was hybridized at 65° C. for 20 hours. The blot was subsequently washed four times for 15 minutes each at 65° C. in a solution containing 15 mM NACl, 1.5 mM Na₃Citrate, 0.1% sodium lauryl sulfate (SDS) and exposed to the phosphoimager screen for quantitation.

Results

SGP012 (SEQ ID NO:11, encoding SEQ ID NO:23) is expressed at the highest levels in the following tissue: testis; cerebellum, right; colon, descending; cerebellum left; lymph node; Burkitt's lymphoma; Daudi; and mammary gland. This pattern of expression suggests that SGP012 may play a role in diseases of the central nervous system (cerebellum expression), in immune system disease (the lymph node; Burkitt's lymphoma, and Daudi are all immune system tissues), or breast cancer (from expression in mammary tissue).

SGP002 (SEQ D NO:2, encoding SEQ ID NO: 14) is expressed at the highest levels in the following tissues: adrenal gland; placenta; prostate; salivary gland; mammary gland; pituitary gland. Expression in the prostate and breast may indicate a role for this phosphatase in cancer of these tissues. Expression in the adrenal gland may indicate a role in metabolic processes controlled by that gland, such as stress response.

### Example 4

### Chromosomal Localization of Mammalian Protein Phosphatases

Several sources were used to find information about the chromosomal localization of the genes in the present invention. First, the accession number for the nucleic acid sequence was used to query the Unigene database. The site containing the Unigene search engine is: http://www.ncbi.nlm.nih.gov/UniGene/Hs.Home.html. Information on map position within the Unigene database is imported from several sources, including the Online Mendelian Inheritance in Man (OMIM, http://www.ncbi.nlm.nih.gov/Omim/searchomim.html), The Genome Database (http://gdb.infobiogen.fr/gdb/simpleSearch.html), and the Whitehead Institute human physical map (http://carbon.wi.mit.edu:8000/cgi-bin/contig/sts_info?database=release). If Unigene has not mapped the EST, then the nucleic acid for the gene of interest is used as a query against databases, such as dbsts and htgs (described at http://www.ncbi.nlm.nih.gov/BLAST/blast_databases.html) containing sequences that have been mapped already. The nucleic acid sequence is searched using BLAST-2 at NCBI (http://www.ncbi.nlm.nih.gov/cgi-bin/BLAST/nph-newblast) and is used to query either dbsts or htgs. Once a cytogenetic region has been identified by one of these approaches, disease association is established by searching OMIM with the cytogenetic location. OMIM maintains a searchable catalog of cytogenetic map locations organized by disease. A thorough search of available literature for the cytogenetic region is also made using Medline (http://www.ncbi.nlm.nih.gov/PubMed/medline.html). References for association of the mapped sites with chromosomal abnormalities found in human cancer can be found in: Knuutila, et al., Am J Pathol, 1998, 152:1107-1123. The results are discussed in the Section on Nucleic Acids above.

### Example 5

### Candidate Single Nucleotide Polymorphisms (SNPs)

Materials and Methods

The most common variations in human DNA are single nucleotide polymorphisms (SNPs), which occur approximately once every 100 to 300 bases. Because SNPs are expected to facilitate large-scale association genetics studies, there has recently been great interest in SNP discovery and direction. Candidate SNPs for the genes in this patent were identified by blastn searching the nucleic acid sequences against the public database of sequences containing documented SNPs (dbSNP, at NCBI, http://www.ncbi.nlm.nih.gov/SNP/snpblastpretty.html). dbSNP accession numbers for the SNP-containing sequences are given. SNPs were also identified by comparing several databases of expressed genes (dbEST, NRNA) and genomic sequence (i.e., NRNA) for single basepair mismatches. The results are shown in Table 2, in the column labeled “SNPs”. These are candidate SNPs—their actual frequency in the human population was not determined. The code below is standard for representing DNA sequence:
G=Guanosine
A=Adenosine
T=Thymidine
C=Cytidine
R=G or A, puRine
Y=C or T, pYrimidine
K=G or T, Keto
W=A or T, Weak (2 H-bonds)
S=C or G, Strong (3 H-bonds)
M=A or C, aMino
B=C, G or T (i.e., not A)
D=A, G or T (i.e., not C)
H=A, C or T (i.e., not G)
V=A, C or G (i.e., not T)
N=A, C, G or T, aNy

X=A, C, G or T

| | |
|---|---|
| complementary | G A T C R Y W S K M B V D H N X |
| DNA | +−+−+−+−+−+−+−+−+−+−+−+−+−+−+−+−+ |
| stands | C T A G Y R S W M K V B H D N X |

For example, if two versions of a gene exist, one with a “C” at a given position, and a second one with a “T: at the same position, then that position is represented as a Y, which means C or T. In table 1, for SGP002, the SNP column says “1165=R”, which means that at position 1165, a polymorphism exists, with that position sometimes containing a G and sometimes an A (R represents A or G). SNPs may be important in identifying heritable traits associated with a gene.

Results

SGP006 has a single nucleotide polymorphism at position 6222: 6222=R (ccaaacataagtggcacar). The dbSNP accession number is rs881179. This SNP occurs in the 3′ untranslated region.

SGP018 has a single nucleotide polymorphism at position 1161; 1161=S (catctaccccaatgas). The dbSNP accession number is ss1765940. This SNP results in a change in the peptide sequence: amino acid number 183 can be either a glutamic acid, when nucleotide 549=G; or amino acid 183 can be an aspartic acid, when nucleotide 549=C. This change is fairly conservative, since both amino acids are acidic, but could alter the biology of the enzyme. A second SNP is silent: 2929=M (agaagatgtctgagtacm) dbSNP|ss1765941, results in a Glycine at amino position 977 with either a C or A at that position.

SGP008 has a single nucleotide polymorphism at position 871: 871=S (cagcagcctccgagggaaccs). The accession number for this SNP in dbSNP is ss1389419. This is a non-silent change, with position 291 either a valine, when nucleotide 871=G, or a leucine, when nucleotide 871=C. This change could alter the biology of the enzyme.

### Example 6

### Isolation of cDNAs Encoding Mammalian Protein Phosphatases

Materials and Methods

Identification of Novel Clones

Total RNAs are isolated using the Guanidine Salts/Phenol extraction protocol of Chomczynski and Sacchi (P. Chomczynsid and N. Sacchi, Anal. Biochem 162, 156 (1987)) from primary human tumors, abnormal and tumor cell lines, normal human tissues, and sorted human hematopoietic cells. These RNAs are used to generate singe-stranded cDNA using the Superscript Preamplification System (GIBCO BRL, Gaithersburg, Md.; Gerard, G F et al. (1989), FOCUS 11, 66) under conditions recommended by the manufacturer. A typical reaction uses 10 μg total RNA with 1.5 μg oligo(dT)₁₂₋₁₈ in a reaction volume of 60 μL. The product is treated with RNaseH and diluted to 100 μL with H₂0. For subsequent PCR amplification, 1-4 μL of this sscDNA is used in each reaction.

Degenerate oligonucleotides are synthesized on an Applied Biosystems 3948 DNA synthesizer using established phosphoramidite chemistry, precipitated with ethanol and used unpurified for PCR. These primers are derived from the sense and antisense strands of conserved motifs within the catalytic domain of several protein phosphatases. Degenerate nucleotide residue designations are: N=A, C, G, or T; R=A or G; Y=C or T; H=A, C or T not G; D=A, G or T not C; S=C or G; and W=A or T.

PCR reactions are performed using degenerate primers applied to multiple single-stranded cDNAs. The primers are added at a final concentration of 5 μM each to a mixture containing 10 mM TrisHCl, pH 8.3, 50 mM KCl 1.5 mM MgCl₂, 200 μM each deoxynucleoside triphosphate, 0.001% gelatin, 1.5 U AmpliTaq DNA Polymerase (Perkin-Elmer/Cetus), and 1-4 μL cDNA. Following 3 min denaturation at 95° C., the cycling conditions are 94° C. for 30 s, 50° C. for 1 min, and 72° C. for 1 min 45 s for 35 cycles. PCR fragments migrating between 300-350 bp are isolated from 2% agarose gels using the GeneClean Kit (Bio101), and T-A cloned into the pCRII vector (Invitrogen Corp. U.S.A.) according to the manufacturer's protocol.

Colonies are selected for mini plasmid DNA-preparations using Qiagen columns and the plasmid DNA is sequenced using a cycle sequencing dye-terminator kit with AmpliTaq DNA Polymerase, FS (ABI, Foster City, Calif.). Sequencing reaction products are run on an ABI Prism 377 DNA Sequencer, and analyzed using the BLAST alignment algorithm (Altschul, S. F. et al., J. Mol. Biol. 215: 403-10).

Additional PCR strategies are employed to connect various PCR fragments or ESTs using exact or near exact oligonucleotide primers. PCR conditions are as described above except the annealing temperatures are calculated for each oligo pair using the formula: Tm=4(G+C)+2(A+T).

Isolation of cDNA Clones:

Human cDNA libraries are probed with PCR or EST figments corresponding to phosphatase-related genes. Probes are ³²P-labeled by random priming and used at 2×10⁶ cpm/mL following standard techniques for library screening. Pre-hybridization (3 h) and hybridization (overnight) are conducted at 42° C. in 5×SSC, 5× Denhart's solution, 2.5% dextran sulfate 50 mM Na₂PO₄/NaHPO₄, pH 7.0, 50% formamide with 100 mg/mL denatred salmon sperm DNA. Stringent washes are performed at 65° C. in 0.1×SSC and 0.1% SDS. DNA sequencing is carried out on both strands using a cycle sequencing dye-terminator kit with AmpliTaq DNA Polymerase, FS (ABI. Foster City, Calif.). Sequencing reaction products are run on an ABI Prism 377 DNA Sequencer.

### Example 7

### Protein Phosphatase Gene Expression

Expression Vector Construction

Expression constructs are generated for some of the human cDNAs including: a) full-length clones in a pCDNA expression vector, b) a GST-fusion construct containing the catalytic domain of the novel phosphatase fused to the C-terminal end of a GST expression cassette; and c) a full-length clone containing a Cys to Ser (C to S) mutation at the predicted catalytic site within the phosphatase domain, inserted in the pCDNA vector.

The “C to S” mutants of the phosphatase might function as dominant negative constructs, and will be used to elucidate the function of these novel phosphatases.

### Example 8

### Generation of Specific Immunoreagents to Protein

Phosphates

Materials and Methods

Specific Immunoreagents are raised in rabbits against KLH- or MAP-conjugated synthetic peptides corresponding to isolated phosphatase polypeptides. C-terminal peptides are conjugated to KLH with glutaraldehyde, leaving a free C-terminus. Internal peptides are MAP-conjugated with a blocked N-terminus. Additional immunoreagents can also be generated by immunizing rabbits with the bacterially expressed GST-fusion proteins containing the cytoplasmic domains of each novel PTP or STP.

The various immune sera are first tested for reactivity and selectivity to recombinant protein, prior to testing for endogenous sources.

Western Blots

Proteins in SDS PAGE are transferred to immobilon membrane. The washing buffer is PBST (standard phosphate-buffered saline pH 7.4+0.1% Triton X-100). Blocking and antibody incubation buffer is PBST+5% milk. Antibody dilutions varied from 1:1000 to 1:2000.

### Example 9

### Recombinant Expression and Biological Assays for Protein Phosphatases

Materials and Methods

Transient Expression of Phosphatases in Mammalian Cells

The pcDNA expression plasmids (10 μg DNA/100 mm plate) containing the STE20-related phosphatase constructs are introduced into 293 cells with lipofectamine (Gibco BRL). After 72 hours, the cells are harvested in 0.5 mL solubilization buffer (20 mM HEPES, pH 7.35, 150 mM NaCl, 10% glycerol 1% Triton X-100, 1.5 mM MgCl₂, 1 mM EGTA, 2 mM phenylmethylsulfonyl fluoride, 1 μg/mL aprotinin). Sample aliquots are resolved by SDS polyacrylamide gel electrophoresis (PAGE) on 6% acrylamide/0.5% bis-acrylamide gels and electrophoretically transferred to nitrocellulose. Non-specific binding is blocked by preincubating blots in Blotto (phosphate buffered saline containing 5% w/v non-fat dried milk and 0.2% v/v Nonidet P-40 (Sigma)), and recombinant protein was detected using the various anti-peptide or anti-GST-fusion specific antisera.

In Vitro Phosphatase Assays

Three days after transfection with the phosphatase expression constructs, a 10 cm an pat of 293 cells is washed with PBS and solubilized on ice with 2 mL PBSTDS containing phosphatase inhibitors (10 mM NaHPO₄, pH 7.25, 150 mM NaCl, 1% Triton X-100, 0.5% deoxycholate, 0.1% SDS, 0.2% sod azide, 1 mM NaF, 1 mM EGTA, 4 mM sodium orthovaadate, 1% aprotinin 5 μg/mL leupeptin). Cell debris is removed by centrifugation (12000×g, 15 min 4° C.) and the lysate is precleared by two successive incubations with 50 μL of a 1:1 slurry of protein A sepharose for 1 hour each. One half mL of the cleared supernatant is reacted with 10 μL of protein A purified phosphatase-specific antisera (generated from the GST fusion protein or antipeptide antisera) plus 50 μl of a 1:1 slurry of protein A-sepharose for 2 h at 4° C. The beads are then washed 2 times in PBSTDS, and 2 times in HNTG (20 mM HEPES, pH 7.5/150 mM NaCl, 0.1% Triton X-100, 10% glycerol).

The immunopurified phosphatases on sepharose beads are resuspended in 20 μL HNTG plus 30 mM MgCl₂, 10 mM MnCl₂, and 20 μCi [α³²P]ATP (3000 Ci/mmol). The phosphatase reactions are run for 30 min at room temperature, and stopped by addition of HNTG supplemented with 50 mM EDTA. The samples are washed 6 times in HNTG, boiled 5 min in SDS sample buffer and analyzed by 6% SDS-PAGE followed by autoradiography. Phosphoamino acid analysis is performed by standard 2D methods on ³²P-labeled bands excised from the SDS-PAGE gel.

Similar assays are performed on bacterially expressed GST-fusion constructs of the phosphatases.

### Example 10

### Demonstration of Gene Amplification by Southern Blotting

Materials and Methods

Nylon membranes are purchased from Boehringer Mannheim. Denaturing solution contains 0.4 M NaOH and 0.6 M NaCl. Neutralization solution contains 0.5 M Tris-HCL, pH 7 and 1.5 M NaCl. Hybridization solution contains 50% formamide, 6×SSPE, 2.5× Denhardt's solution, 0.2 mg/mL denatured salmon DNA, 0.1 mg yeast tRNA, and 0.2% sodium dodecyl sulfate. Restriction enzymes are purchased from Boehringer Mannheim. Radiolabeled probes are prepared using the Prime-it II kit by Stratagene. The beta-actin DNA fragment used for a probe template is purchased from Clontech.

Genomic DNA is isolated from a variety of tumor cell lines (such as MCF-7, MDA-MB-231, Calu6, A549, HCT-15, HT-29, Colo 205, LS-180, DLD-1, HCT-116, PC3, CAPAN-2, MA-PaCa-2, PANC-1, AsPc-1, BxPC-3, OVCAR-3, SKOV3, SW 626 and PA-1, and from two normal cell lines.

A 10 μg aliquot of each genomic DNA sample is digested with EcoR I restriction enzyme and a separate 10 μg sample is digested with Hind III restriction enzyme. The restriction-digested DNA samples are loaded onto a 0.7% agarose gel and, following electrophoretic separation, the DNA is capillary-transferred to a nylon membrane by standard methods (Sambrook, J. et al (1989) Molecular Cloning: A Laboratory Manual, Cold Spring Harbor Laboratory).

### Example 11

### Detection Of Protein-Protein Interaction Through Phage Display

Materials and Methods

Phage display provides a method for isolating molecular interactions based on affinity for a desired bait. cDNA fragments cloned at fusions to phage coat proteins are displayed on the sure of the phage. Phage(s) interacting with a bait are enriched by affinity purification and the insert DNA from individual clones is analyzed.

T7 Phage Display Libraries

All libraries are constructed in the T7Select1-1b vector (Novagen) according to the manufacturer's directions.

Bait Presentation

Protein domains to be used as baits are generated as C-terminal fusions to GST and expressed in E. coli. Peptides are chemically synthesized and biotinylated at the N-terminus using a long chain spacer biotin reagent.

Selection

Aliquots of refreshed libraries (10¹⁰-10¹² pfu) supplemented with PanMix and a cocktail of E. coli. inhibitors (Sigma P-8465) are incubated for 1-2 hrs at room temperature with the immobilized baits. Unbound phage is extensively washed (at least 4 times) with wash buffer.

After 3-4 rounds of selection, bound phage is eluted in 100 μL of 1% SDS and plated on agarose plates to obtain single plaques.

Identification of Insert DNAs

Individual plaques are picked into 25 μL of 10 mM EDTA and the phage is disrupted by heating at 70° C. for 10 min 2 μL of the disrupted phage are added to 50 μL PCR reaction mix. The insert DNA is amplified by 35 rounds of thermal cycling (94° C., 50 sec; 50° C., 1 min; 72° C., 1 min).

Composition of Buffer

10× PanMix

5% Triton X-100

10% non-fat dry milk (Caration)

10 mM EGTA

250 mM NaF

250 μg/mL Heparin (sigma)

250 μg/mL sheared, boiled salmon sperm DNA (sigma)

0.05% Na azide

Prepared in PBS

Wash Buffer

PBS supplemented with:

0.5% NP-40

25 μg/mL heparin

PCR reaction mix

1.0 mL 10×PCR buffer (Perkin-Elmer, with 15 mM Mg)

0.2 mL each dNTPs (10 mM stock)

0.1 mL T7UP primer (15 pmol/μL) GGAGCTGTCGTATTCCAGTC

0.1 mL T7DN primer (15 pmol/μL) AACCCCTCAAGACCCGTTTAG

0.2 mL 25 mM MgCl₂ or MgSO₄ to compensate for EDTA

Q.S. to 10 mL with distilled water

Add 1 unit of Taq polymerase per 50 μL reaction

Library: T7 Select1-H441

### Compound Evaluation

It will be appreciated that, in any given series of compounds, a spectrum of biological activity will be observed. In a preferred embodiment, the present invention relates to compounds demonstrating the ability to modulate protein enzymes related to cellular signal transduction; preferably, protein phosphatases; and most preferably, protein tyrosine phosphatases. The assays described below are employed to select those compounds demonstrating the optimal degree of the desired activity.

As used herein, the phrase “optimal degree of desired activity” refers to the highest therapeutic index, defined above, against a protein enzyme which mediates cellular signal transduction and which is related to a particular disorder so as to provide an animal or a human patient, suffering from such disorder with a therapeutically effective amount of a compound of this invention at the lowest possible dosage.

### Assays For Determining Inhibitory Activity

Various procedures known in the art may be used for identifying, evaluating or assaying the inhibition of activity of protein enzymes, in particular protein phosphatases, by the compounds of the invention. For example but without limitation, with regard to phosphatases such assays involve exposing target cells in culture to the compounds and (a) biochemically analyzing cell lysates to assess the level and/or identity of phosphorylated proteins; or (b) scoring phenotypic or functional changes in treated cells as compared to control cells that were not exposed to the test substance.

Where mimics of the natural ligand for a signal transducing receptor are to be identified or evaluated, the cells are exposed to the compound of the invention and compared to positive controls which are exposed only to the natural ligand, and to negative controls which are not exposed to either the compound or the natural ligand. For receptors that are known to be phosphorylated a basal level in the absence of the natural ligand, such as the insulin receptor, the assay may be carried out in the absence of the ligand. Where inhibitors or enhancers of ligand induced signal transduction are to be identified or evaluated, the cells are exposed to the compound of the invention in the presence of the natural ligand and compared to controls which are not exposed to the compound of the invention.

The assays described below may be used as a primary screen to evaluate the ability of the compounds of this invention to inhibit phosphatase activity of the compounds of the invention. The assays may also be used to assess the relative potency of a compound by testing a range of concentrations, in a range from 100 μM to 1 pM, for example, and computing the concentration at which the amount of phosphorylation or signal transduction is reduced or increased by 50% (IC50) compared to controls.

### Biochemical Assays

In one embodiment target cells having a substrate molecule that is phosphorylated or dephosphorylated on a tyrosine residue during signal transduction are exposed to the compounds of the invention and radiolabelled phosphate, and thereafter, lysed to release cellular contents, including the substrate of interest. The substrate may be analyzed by separating the protein components of the cell lysate using a sodium dodecyl sulphate-polyacrylamide gel electrophoresis (SDS-PAGE) technique, in either one or two dimensions, and detecting the presence of phosphorylated proteins by exposing to X-ray film. In a similar technique, but without radioactive labeling, the protein components separated by SDS-PAGE are transferred to a nitrocellulose membrane, the presence of pTyr is detected using an antiphosphotyrosine (anti-pTyr) antibody. Alternatively, it is preferred that the substrate of interest be first isolated by incubating the cell lysate with a substrate-specific anchoring antibody bound to a solid support and thereafter, washing away non-bound cellular components, and assessing the presence or absence of pTyr on the solid support by an anti-pTyr antibody. This preferred method can readily be performed in a microtiter plate format by an automated robotic system, allowing for testing of large numbers of samples within a reasonably short time frame.

The anti-pTyr antibody can be detected by labeling it with a radioactive substance which facilitates its detection by autoradiography. Alternatively, the anti-pTyr antibody can be conjugated with an enzyme, such as horseradish peroxidase, and detected by subsequent addition of an appropriate substrate for the enzyme, the choice of which would be clear to one skilled in the art. A further alternative involves detecting the anti-pTyr antibody by reacting with a second antibody which recognizes the anti-pTyr antibody, this second antibody being labeled with either a radioactive substance or an enzyme as previously described. Any other methods for the detection of an antibody known in the art may be used.

The above methods may also be used in a cell-free system wherein cell lysate containing the signal-transducing substrate molecule and phosphatase is mixed with a compound of the invention and a kinase. The substrate is phosphorylated by initiating the kinase reaction by the addition of adenosine triphosphate (ATP). To assess the activity of the compound, the reaction mixture may be analyzed by the SDS-PAGE technique or it may be added to a substrate-specific anchoring antibody bound to a solid support, and a detection procedure as described above is performed on the separated or captured substrate to assess the presence or absence of pTyr. The results are compared to those obtained with reaction mixtures to which the compound is not added. The cell-free system does not request the natural ligand or knowledge of its identity. For example, Posner et al. (U.S. Pat. No. 5,155,031) describes the use of insulin rector as a substrate and rat adipocytes as target cells to demonstrate the ability of pervanadate to inhibit PTP activity. Burke et al., 1994, Biochem. Biophys. Res. Comm., 204:129-134) describes the use of autophosphorylated insulin receptor and recombinant PTP1B in assessing the inhibitory activity of a phosphotyrosyl mimetic.

In addition to measuring phosphorylation or dephosphorylation of substrate proteins, activation or modulation of second messenger production, changes in cellular ion levels, association, dissociation or translation of signaling molecules, gene induction or transcription or translation of specific genes may also be monitored. These biochemical assays may be performed using conventional techniques developed for these purposes.

### Biological Assays

The ability of the compounds of this invention to modulate the activity of PTPs, which control signal transduction, may also be measured by scoring for morphological or functional changes associated with ligand binding. Any qualitative or quantitative techniques known in the art may be applied for observing and measuring cellular processes which come under the control of phosphatases in a signaling pathway. Such cellular processes may include, but are not limited to, anabolic and catabolic processes, cell proliferation, cell differentiation, cell adhesion, cell migration and cell death.

The techniques that have been used for investigating the various biological effects of vanadate as a phosphatase inhibitor may be adapted for use with the compounds of the invention. For example, vanadate has been shown to activate an insulin-sensitive facilitated transport system for glucose and glucose analogs in rat adipocytes (Dubyak et al., 1980, J. Biol. Chem., 256:5306-5312). The activity of the compounds of the invention may be assessed by measuring the increase in the rate of transport of glucose analog such as 2deoxy-³H-glucose in rat adipocytes that have been exposed to the compounds. Vanadate also mimics the effect of insulin on glucose oxidation in rat adipocytes (Shechter et al., 1980, Nature, 284:556-558). The compounds of this invention may be tested for simulation of glucose oxidation by measuring the conversion of ¹⁴C-glucose to ¹⁴CO₂. Moreover, the effect of sodium orthovanadate on erythropoietin-mediated cell proliferation has been measured by cell cycle analysis based on DNA content as estimated by incorporation of tritiated thymidine during DNA synthesis (Spivak et al., 1992, Exp. Hematol., 20:500-504). Likewise, the activity of the compounds of this invention toward phosphatases that play a role in cell proliferation may be assessed by cell cycle analysis.

The activity of the compounds of this invention can also be assessed in animals using experimental models of disorders caused by or related to dysfunctional signal transduction. For example, the activity of a compound of this invention may be tested for its effect on insulin receptor signal transduction in non-obese diabetic mice (Lund et al., 1990, Nature, 345:727-729), B B Wistar rats and streptozotocin-induced diabetic rats (Solomon et al., 1989, Am. J. Med Sci., 297:372-376). The activity of the compounds may also be assessed in animal carcinogenesis experiments since phosphatases can play an important role in dysfunctional signal transduction leading to cellular transformation. For example, okadaic acid, a phosphatase inhibitor, has been shown to promote tumor formation on mouse skin (Suganuma et al., 1988, Proc. Natl. Acad. Sci, 85:1768-1771).

The data obtained from these cell culture assays and animal studies can be used in formulating a range of dosages for use in humans. The dosage of the compounds of the invention should lie within a range of circulating concentrations with little or no toxicity. The dosage may vary within this range depending on the dosage form employed and the route of administration.

Phosphotyrosine Enzyme Linked Immunosorbent Assay

This assay may be used to test the ability of the compounds of the invention to inhibit dephosphorylation of phosphotyrosine (pTyr) residues on insulin receptor (IR). Those skilled in the art will recognize that other substrate molecules, such as platelet derived growth factor receptor, may be used in the assay by using a different target cell and anchoring antibody. By using different substrate molecules in the assay, the activities of the compounds of this invention toward different protein tyrosine enzymes may be assessed. In the case of IR, an endogenous kinase activity is active at low level even in the absence of insulin. Thus, no insulin is needed to stimulate phosphorylation of IR. That is, after exposure to a compound, cell lysates can be prepared and added to microtiter plates coated with anti-insulin receptor antibody. The level of phosphorylation of the capture insulin receptor is detected using an anti-pTyr antibody and an enzyme-linked secondary antibody.

Assay Methods in Determination of Compound-PTP IC50

The following in vitro assay procedure is preferred to determine the level of activity and effect of the different compounds of the present invention on one or more of the PTPs. Similar assays can be designed along the same lines for any PTP using techniques well known in the art.

The catalytic assays described herein are performed in a 96-well format. The general procedure begins with the determination of PTP optimal pH using a three-component buffer system that minimizes ionic strength variations across a wide range of buffer pH. Next, the Michaelis-Menten constant, or Km, is determined for each specific substrate-PTP system. This Km value is subsequently used as the substrate reaction concentration for compound screening. Finally, the test PTP is exposed to varying concentrations of compound for fifteen minutes and allowed to react with substrate for ten minutes. The results are plotted as percent inhibition versus compound concentration and the IC50 interpolated from the plot.

The following materials and reagents are used

1. Assay Buffer is used as solvent for all assay solutions unless otherwise indicated.

| Component | Concentration |
|---|---|
| Acetate (Fisher Scientific A38-500) | 100 mM |
| Bis-Tris (Sigma B-7535) | 50 mM |
| Tri's (Fisher Scientific BP152-5) | 50 mm |
| Glycerol (Fisher Scientific BP229-1) | 10% (v/v) |

*1 mM DTT is added immediately prior to use
2. 96 Well Easy Wash Plate (Costar 3369)
3. p-Nitrophenyl Phosphate (Boehringer Mannheim 738-379)
4. Fluorescein Diphosphate (Molecular Probes F-2999)
5. 0.22 μm Stericup Filtration System 500 ml (Millipore SCGPU05RE)
6. 10N NaOH (Fisher Scientific SS255-1)
7. 10N HCl (Fisher Scientific A144-500)
8. Compounds were dissolved in DMSO (Sigma D-5879) at 5 or 10 mM concentrations and stored at −20° C. in small aliquots.
   Methods:

All assays are performed using pNPP or FDP as substrate. The optimum pH is determined for each PTP used.

PTP Assay

PTPase activity is assayed at 25° C. in a 100-μl reaction mixture containing an appropriate concentration of pNPP or FDP as substrate. The reaction is initiated by addition of the PTP and quenched after 10 min by addition of 50 μl of 1N NaOH. The non-enzymatic hydrolysis of the substrate is corrected by measuring the control without the addition of the enzyme. The amount of p-nitrophenol produced is determined from the absorbance at 410 nm. To determine the kinetic parameter, Km, the initial velocities are measured at various substrate concentrations and the data are fitted to the Michaelis equation where velocity=(Vmax*[S])/(Km+[S]), and [S]=substrate reaction concentration.

Inhibition Studies

The effect of the compounds on PTP is evaluated at 25° C. using pNPP or FDP as substrate. PTP is pre-incubated for fifteen minutes with various concentrations of compound. Substrate is then added at a fixed concentration (usually equal to the Km previously calculated). After 10 minutes, NaOH is added to stop the reaction. The hydrolysis of pNPP is followed at 410 nm on the Biotek Powerwave 200 microplate scanning spectrophotometer. The percent inhibition is calculated as follows: Percent Inhibition=[(control signal−compound signal)/control signal]×100%. The IC50 is then deteined by interpolation of a percent inhibition versus compound concentration plot.

Plasmids designed for bacterial GST-PTP fusion protein expression are derived by insertion of PCR-generated human PTP fragments into pGEX vectors (Pharmacia Biotech). Several of these constructs are then used to subclone phosphatases into pFastBac-1 for example on in Sf-9 insect cells. Oligonucleotides that are used for the initial amplification of PTP genes are shown below. The cDNAs are prepared using the Gilbo BRL superscript preamplification system on RNAs purchased from Clontech.

### Conclusion

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that Me invention is also thereby described in terms of any individual member or subgroup of members of the Markush group. For example, if X is described as selected from the group consisting of bromine, chlorine, and iodine, claims for X being bromine and claims for X being bromine and chlorine are fully described.

In view of the degeneracy of the genetic code, other combinations of nucleic acids also encode the claimed peptides and proteins of the invention. For example, all four nucleic acid sequences GCT, GCC, GCA, and GCG encode the amino acid alanine. Therefore, if for an amino acid there exists an average of three codons, a polypeptide of 100 amino acids in length will on average, be encoded by 3100, or 5×1047, nucleic acid sequences. Thus, a nucleic acid sequence can be modified to form a second nucleic acid sequence, encoding the same polypeptide as encoded by the first nucleic acid sequences, using routine procedures and without undue experimentation. Thus, all possible nucleic acids that encode the claimed peptides and proteins are also fully described herein, as if all were written out in full taking into account the codon usage, especially that preferred in humans. Furthermore, changes in the amino acid sequences of polypeptides, or in the corresponding nucleic acid sequence encoding such polypeptide, may be designed or selected to take place in an area of the sequence where the significant activity of the polypeptide remains unchanged. For example, an amino acid change may take play within a β-turn, away from the active site of the polypeptide. Also changes such as deletions (e.g. removal of a segment of the polypeptide, or in the corresponding nucleic acid sequence encoding such polypeptide, which does not affect the active site) and additions (e.g. addition of more amino acids to the polypeptide sequence without affecting the function of the active site, such as the formation of GST-fusion proteins, or additions in the corresponding nucleic acid sequence encoding such polypeptide without affecting the function of the active site) are also within the scope of the preset invention. Such changes to the polypeptides can be performed by those with ordinary skill in the art using routine procedures and without undue experimentation. Thus, all possible nucleic and/or amino acid sequences that can readily be determined not to affect a significant activity of the peptide or protein of the invention are also fully described herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims.

1. An isolated, enriched or purified nucleic acid molecule encoding a phosphatase polypeptide, wherein said nucleic acid molecule:
(a) encodes a polypeptide having an amino acid sequence set forth in SEQ ID NO:23 or SEQ ID NO:24;
(b) is the complement of the nucleic acid molecule of (a);
(c) hybridizes under conditions that prevent hybridization of nucleic acids having more than 2 mismatches out of 20 contiguous nucleotides, to the nucleic acid molecule of (a) and encodes a naturally occurring phosphatase polypeptide;
(d) encodes a polypeptide having an amino acid sequence set forth in SEQ ID NO:23 or SEQ ID NO:24, except that said polypeptide lacks one or more, but not all, of the following regions: an N-terminal domain, a C-terminal catalytic domain, a catalytic domain, a C-terminal domain, a coiled-coil structure region, a proline-rich region, a spacer region and a C-terminal tail; or
(e) is the complement of the nucleotide sequence of (d).

2. A vector, comprising the nucleic acid molecule of claim 1 and, optionally a promoter effective to initiate transcription in a host cell.

3. The nucleic acid molecule of claim 1, wherein said nucleic acid molecule is isolated, enriched, or purified from a mammal.

4. The nucleic acid molecule of claim 3, wherein said mammal is a human.

5. A probe, comprising a nucleic acid that hybridizes, under conditions that prevent hybridization of nucleic acids having more than 2 mismatches out of 20 contiguous nucleotides, to the nucleic acid of claim 1.

6. A recombinant cell comprising the nucleic acid molecule of claim 1 encoding a phosphatase polypeptide having an amino acid sequence set forth in SEQ ID NO:23 or SEQ ID NO:24.

7. An isolated, enriched, or purified phosphatase polypeptide, wherein said polypeptide comprises an amino acid sequence having
(a) an amino acid sequence set forth in SEQ ID NO:23 or SEQ ID NO:24; or
(b) an amino acid sequence forth in SEQ ID NO:23 or SEQ ID NO:24, except that the polypeptide lacks one or more, but not all, of the following regions: an N-terminal domain, a C-terminal catalytic domain, a catalytic domain, a C-terminal domain, a coiled-coil structure region, a proline-rich region, a spacer region and a C-terminal tail.

8. The phosphatase polypeptide of claim 7, wherein said polypeptide is isolated, purified, or enriched from a mammal.

9. The phosphatase polypeptide of claim 8, wherein said mammal is a human.

10. An antibody or antibody fragment having specific binding affinity to a phosphatase polypeptide or to a domain of said polypeptide, wherein said polypeptide is a phosphatase polypeptide having an amino acid sequence set forth in SEQ ID NO:23 or SEQ ID NO:24.

11. A hybridoma which produces an antibody having specific binding affinity to a phosphatase polypeptide having an amino acid sequence set forth in SEQ ID NO:23 or SEQ ID NO:24.

12. A kit comprising an antibody which binds to a polypeptide of claim 7 or 8 and negative control antibody.

13. A method for identifying a substance that modulates the activity of a phosphatase polypeptide comprising the steps of:
(a) contacting the phosphatase polypeptide having an amino acid sequence set forth in SEQ ID NO:23 or SEQ ID NO:24 with a test substance;
(b) measuring the activity of said polypeptide; and
(c) determining whether said substance modulates the activity of said polypeptide.

14. A method for identifying a substance that modulates the activity of a phosphatase polypeptide in a cell comprising the steps of:
(a) expressing a phosphatase polypeptide having an amino acid sequence set forth in SEQ ID NO:23 or SEQ ID NO:24;
(b) adding a test substance to said cell; and
(c) monitoring a change in cell phenotype or the interaction between said polypeptide and a natural binding partner.

15. A method for treating a disease or disorder by administering to a patient in need of such treatment a substance that modulates the activity of a phosphatase having an amino acid sequence selected from the group consisting of those set forth in SEQ ID NO:23 or SEQ ID NO:24.

16. The method of claim 15, wherein said disease or disorder is selected from the group consisting of cancers, immune-related diseases and disorders, cardiovascular disease, brain or neuronal-associated diseases, and metabolic disorders.

17. The method of claim 15, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; ocular diseases, metabolic disorders, and diabetes.

18. The method of claim 15, wherein said disease or disorder is selected from the group consisting of migraines; pain; sexual dysfunction; mood disorders; attention disorders; cognition disorders; hypotension; hypertension; psychotic disorders; neurological disorders; dyskinesias; metabolic disorders; and organ transplant rejection.

19. The method of claim 15, wherein said substance modulates phosphatase activity in vitro.

20. The method of claim 19, wherein said substance is a phosphatase inhibitor.

21. A method for detection of a phosphatase polypeptide in a sample as a diagnostic tool for a disease or disorder, wherein said method comprises:
(a) contacting said sample with a nucleic acid probe which hybridizes under hybridization assay conditions to a nucleic acid target region of a phosphatase polypeptide having an amino acid sequence selected from the group consisting of those set forth in SEQ ID NO:23 or SEQ ID NO:24, said probe comprising the nucleic acid sequence encoding said polypeptide, fragments thereof, or the complements of said sequences and fragments; and
(b) detecting the presence or amount of the probe:target region hybrid as an indication of said disease.

22. The method of claim 21, wherein said disease or disorder is selected from the group consisting of cancers, immune-related diseases and disorders, cardiovascular disease, brain or neuronal-associated diseases, and metabolic disorders.

23. The method of claim 21, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; and ocular diseases.

24. The method of claim 21, wherein said disease or disorder is selected from the group consisting of migraines, pain; sexual dysfunction; mood disorders; attention disorders; cognition disorders; hypotension; hypertension; psychotic disorders; neurological disorders; dyskinesias; metabolic disorders; and organ transplant rejection.

25. A method for detection of a phosphatase polypeptide in a sample as a diagnostic tool for a disease or disorder, wherein said method comprises:
(a) comparing a nucleic acid target region encoding said phosphatase polypeptide in a sample, wherein said phosphatase polypeptide has an amino acid sequence set forth in SEQ ID NO:23 or SEQ ID NO:24, or one or more fragments thereof, with a control nucleic acid target region encoding said phosphatase polypeptide, or one or more fragments thereof; and
(b) detecting differences in sequence or amount between said target region and said control target region, as an indication of said disease or disorder.

26. The method of claim 25, wherein said disease or disorder is selected from the group consisting of cancers, immune-related diseases and disorders, cardiovascular disease, brain or neuronal-associated diseases, and metabolic disorders.

27. The method of claim 25, wherein said disease or disorder is selected from the group consisting of cancers of tissues; cancers of hematopoietic origin; diseases of the central nervous system; diseases of the peripheral nervous system; Alzheimer's disease; Parkinson's disease; multiple sclerosis; amyotrophic lateral sclerosis; viral infections; infections caused by prions; infections caused by bacteria; infections caused by fungi; and ocular diseases.

28. The method of claim 25, wherein said disease or disorder is selected from the group consisting of migraines, pain; sexual dysfunction; mood disorders; attention disorders; cognition disorders; hypotension; hypertension; psychotic disorders; neurological disorders; dyskinesias; metabolic disorders; and organ transplant rejection.

29. A nucleic acid that encodes a mammalian phosphatase or a fragment thereof selected from the group consisting of SEQ ID NO:11, and SEQ ID NO: 12.
